SEQUENCE LISTING (2 sequences)

SEQ ID NO: 1 - DNA, 1175 bp, Homo sapiens
  misc_feature 1..1175: note = human cDNA sequence, ENST00000446922.6 HMGB2-203 cdna:protein_coding
  source 1..1175: mol_type = other DNA
gcaaaacaagaggctgaggattgcgttagagataaaccagttcacgccggagccccgtgagggaagcgtctccgttgggtccggccgctctgcgggactctgaggaaaagctcgcaccaggcaagaataccctccaataccctcgggtggacgcggatctgtcaacatgggtaaaggagaccccaacaagccgcggggcaaaatgtcctcgtacgccttcttcgtgcagacctgccgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataaatggctatcctttaatgatgcgtgtggaatgtgtgtgtgtgctcaggcaattattttgctaagaatgtgaattcaagtgcagctcaatactagcttcagtataaaaactgtacagatttttgtatagctgataagattctctgtagagaaaatacttttaaaaaatgcaggttgtagctttttgatgggctactcatacagttagattttacagcttctgatgttgaatgttcctaaatatttaatggtttttttaatttcttgtgtatggtagcacagcaaacttgtaggaattagtatcaatagtaaattttgggttttttaggatgttgcatttcgtttttttaaaaaaaattttgtaataaaattatgtatatta

SEQ ID NO: 2 - protein, 209 aa, unidentified
  REGION 1..209: note = Polypeptide encoded by HMGB2-inhibiting or HMGB2-expressing nucleic acid sequence
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

(19) United States
(12) Patent Application Publication
(10) Pub. No.: US 2025/0115652 A1
(43) Pub. Date: Apr. 10, 2025

(54) COMPOSITIONS AND METHODS FOR MODULATING T CELL RESPONSES

(71) Applicant: The Regents of the University of California, Oakland, CA (US)

(72) Inventors: Roberto TINOCO, Irvine, CA (US); Emily NEUBERT, Irvine, CA (US)

(21) Appl. No.: 18/621,640

(22) Filed: Mar. 29, 2024

Related U.S. Application Data
(60) Provisional application No. 63/455,663, filed on Mar. 30, 2023.

(51) Int. Cl. C07K 14/52; A61K 35/17; A61K 38/00; C12N 15/113; C12N 15/86

(52) U.S. Cl. CPC C07K 14/52; A61K 35/17; C12N 15/1136; C12N 15/86; A61K 38/00; C12N 2750/14143

(57) ABSTRACT

In alternative embodiments, provided are compositions, including products of manufacture and kits, and methods, for increasing HMGB2 (high-mobility group box2) activity in a T cell, for: increasing the rate or amount of differentiation and stemness of memory and exhausted CD8⁺ T cells in vivo, increasing the rate or amounts of effector and memory CD8⁺ T cells in vivo; expanding a subset of progenitor stem cells within an exhausted T cell population in vivo or ex vivo; boosting or increasing CD8⁺ T cells during acute viral infection in an individual in need thereof, increasing memory T cells and memory T cells differentiation, memory precursor effector (MPEC) and central memory (Tcm) T cell phenotypes, and memory recall responses in vivo, increasing the ability of an individual in need thereof to recover from a viral infection, and/or treating, ameliorating, slowing the progression of, decreasing the severity of symptoms of, an infection, optionally, a viral infection, in an individual in need thereof. In alternative embodiments, provided are compositions, including products of manufacture and kits, and methods, for decreasing HMGB2 (high-mobility group box2) activity in a T cell, for: decreasing the rate or amount of differentiation and stemness of memory and exhausted CD8⁺ T cells in vivo, decreasing the rate or amounts of effector and memory CD8⁺ T cells in vivo; expanding a subset of progenitor stem cells within an exhausted T cell population in vivo or ex vivo; and/or treating, ameliorating, slowing the progression of, decreasing the severity of symptoms of, an autoimmune disease, in an individual in need thereof.

### RELATED APPLICATIONS

This U.S. utility patent application claims the benefit of priority under 35 U.S.C. § 119(e) to U.S. Provisional Patent Application Ser. No. (USSN) 63/455,746, Mar. 30, 2023. The aforementioned application is expressly incorporated herein by reference in their entirety and for all purposes.

### STATEMENT AS TO FEDERALLY SPONSORED RESEARCH

This invention was made with government support under GM055246, OD010794, OD021718 and RR025496, awarded by the National Institutes of Health (NIH). The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 28, 2024, is named “5825.151867.xml” and is 4,898 bytes in size. The sequence listing contained in this .XML file is part of the specification and is hereby incorporated by reference herein in its entirety.

### TECHNICAL FIELD

This invention generally relates to immunology and medicine. In alternative embodiments, provided are compositions, including products of manufacture and kits, and methods, for manipulating, and in particular, augmenting or increasing T cell (such as effector CD8⁺ T cell) activity in vitro and in vivo, by for example by increasing HMGB2 (high-mobility group box2) activity in the T cell, or effector CD8⁺ T cell. In alternative embodiments, provided are compositions, including products of manufacture and kits, and methods, for increasing HMGB2 (high-mobility group box2) activity in a T cell, for: increasing the rate or amount of differentiation and stemness of memory and exhausted CD8⁺ T cells in vivo, increasing the rate or amounts of effector and memory CD8⁺ T cells in vivo; expanding a subset of progenitor stem cells within an exhausted T cell population in vivo or ex vivo; boosting or increasing CD8⁺ T cells during acute viral infection in an individual in need thereof, increasing memory T cells and memory T cells differentiation, memory precursor effector (MPEC) and central memory (Tcm) T cell phenotypes, and memory recall responses in vivo, increasing the ability of an individual in need thereof to recover from a viral infection, and/or treating, ameliorating, slowing the progression of, decreasing the severity of symptoms of, an infection, optionally, a viral infection, in an individual in need thereof. In alternative embodiments, provided are compositions, including products of manufacture and kits, and methods, for decreasing HMGB2 (high-mobility group box2) activity in a T cell, for: decreasing the rate or amount of differentiation and stemness of memory and exhausted CD8⁺ T cells in vivo, decreasing the rate or amounts of effector and memory CD8⁺ T cells in vivo; expanding a subset of progenitor stem cells within an exhausted T cell population in vivo or ex vivo; and/or treating, ameliorating, slowing the progression of, decreasing the severity of symptoms of, an autoimmune disease, in an individual in need thereof. In alternative embodiments, HMGB2 expression is modulated to treat cancer in any immunotherapy setting to change the quantity and quality of the responding T cells. In alternative embodiments, HMGB2 is modulated to increase the efficacy of vaccination strategies against any antigen, for example, for a cancer vaccine, a pathogen vaccine, and the like.

### BACKGROUND

During chronic viral infections there is a dynamic interplay between host and pathogen, where multiple cellular and molecular mechanisms inhibit the immune response and facilitate viral persistence. A key mechanism is the differentiation of exhausted T cells, which are dysfunctional and fail to clear the virus. Despite being less functional than effector CD8⁺ T cells, exhausted T cells still provide some protection to the host^{1, 2}, which is highlighted in simian immunodeficiency virus studies showing host progression to AIDS-like disease and death when T cells are depleted³. Chronic antigen stimulation results in responding T cell dysfunction and heterogeneity, with altered transcription, epigenome, and metabolism unique to exhausted T cells^{4, 5, 6, 7, 8}, Two key exhausted T cell subsets, defined by phenotype and transcription factor expression, are progenitor exhausted (Tpex) and terminal exhausted (Tex) T cells^{9, 10, 11}. Tpex cells are long-lived, self-renew, and give rise to Tex cells¹¹. They also express the key transcription factors TCF-1, BCL-6 and BACH2¹². In contrast, Tex cells express high TOX, BLIMP-1, and TIM-3, have increased effector functions, and undergo higher rates of apoptosis¹². Despite detailed characterization of these two main subsets, their differentiation mechanisms have not been fully described.

Understanding exhausted T cell heterogeneity has important clinical implications for immune checkpoint blockade (ICB) therapy against chronic viral infections and cancer¹³. Studies have shown anti-PD-1/anti-PD-L1 blockade reinvigorates the Tpex population, which proliferates and further increases numbers of the more cytotoxic Tex cells¹⁴. Emerging evidence also suggests Tpex cell frequencies may predict patient responsiveness to ICB therapy^{9, 10, 15} and ability to control HIV viremia¹⁶. Importantly, exhausted T cells undergo unique epigenetic changes during differentiation, including permanent marks which sustain their exhausted state¹⁷. Therefore, although exhausted T cells can be re-invigorated with ICB therapy, they revert back to their exhausted phenotype and thereby provide only temporary clinical response in some patients^{18, 19, 20, 21, 22, 23}. Identifying mechanisms of exhausted T cell differentiation and the associated epigenetic changes remains of high clinical interest as these cells may need to be reprogrammed transcriptionally and/or epigenetically to improve immunotherapy efficacy.

HMGB2 is a member of the high-mobility group box (HMGB) family, which are relatively abundant and highly conserved DNA-binding proteins that modify chromatin structure and regulate gene transcription and transcription factor binding^{24, 25, 26}. HMGB2 has known roles in regulating stem cells during various differentiation programs, including myogenesis, spermatogenesis, and neurogenesis^{27, 28, 29}. In mice, Hmgb2 is expressed early in embryogenesis, but is limited to lymphoid organs and testes in adults²⁶. Despite its characterization in numerous cell types, the role of HMGB2 in CD8⁺ T cells has not been investigated. Previous RNA-sequencing analyses found increased Hmgb2 expression in murine exhausted CD8⁺ T cells during lymphocytic choriomeningitis virus (LCMV) infection³⁰ and increased HMGB2 expression in CD8⁺ T cells from cancer patients^{31, 32, 33, 34}.

### SUMMARY

In alternative embodiments, provided are methods for:
increasing the rate or amount of differentiation and stemness of memory and exhausted CD8⁺ T cells in vivo,
increasing the rate or amounts of effector and memory CD8⁺ T cells in vivo;
expanding a subset of progenitor stem cells within an exhausted T cell population in vivo or ex vivo;
boosting or increasing CD8⁺ T cells during acute viral infection in an individual in need thereof,
increasing memory T cells and memory T cells differentiation, memory precursor effector (MPEC) and central memory (Tcm) T cell phenotypes, and memory recall responses in vivo,
increasing the ability of an individual in need thereof to recover from a viral infection, and/or
treating, ameliorating, slowing the progression of, decreasing the severity of symptoms of, an infection, optionally, a viral infection, in an individual in need thereof,
   comprising:
comprising administering to an individual in need thereof:
a nucleic acid encoding a HMGB2 (high-mobility group box2) protein, or
a T cell, optionally a CD8⁺ T cell, genetically manipulated or engineered to have increased HMGB2 activity,
wherein optionally the HMGB2-expressing nucleic acid comprises the nucleic acid sequence (SEQ ID NO:1):
or a nucleic acid having between about 90% to 99% sequence identity to SEQ ID NO:1,
and optionally the HMGB2-expressing nucleic acid comprises a nucleic acid sequence that encodes the polypeptide (SEQ ID NO:2):
or a polypeptide having between about 90% to 99% sequence identity to SEQ ID NO:2, or a polypeptide having about 92%, 95% or 97% sequence identity to SEQ ID NO: 2.

In alternative embodiments, provided are methods for:
decreasing the rate or amount of differentiation and stemness of memory and exhausted CD8⁺ T cells in vivo,
decreasing the rate or amounts of effector and memory CD8⁺ T cells in vivo;
expanding a subset of progenitor stem cells within an exhausted T cell population in vivo or ex vivo; and/or
treating, ameliorating, slowing the progression of, decreasing the severity of symptoms of, an autoimmune disease, in an individual in need thereof,
   comprising:
comprising administering to an individual in need thereof:
a nucleic acid or HMGB2-inhibiting molecule capable of decreasing the activity or expression of a HMGB2 (high-mobility group box2) protein, or
wherein optionally the HMGB2-expressing nucleic acid comprises the nucleic acid sequence (SEQ ID NO:1):
or a nucleic acid having between about 90% to 99% sequence identity to SEQ ID NO:1,
and optionally the HMGB2-expressing nucleic acid comprises a nucleic acid sequence that encodes the polypeptide (SEQ ID NO:2):
or a polypeptide having between about 90% to 99% sequence identity to SEQ ID NO:2, or a polypeptide having about 92%, 95% or 97% sequence identity to SEQ ID NO:2.

In alternative embodiments of methods as provided herein:
the HMGB2-encoding nucleic acid, or the HMGB2-inhibiting nucleic acid, is contained in an expression construct, a plasmid, an expression vehicle, a virus or a vector, and the expression construct, plasmid, expression vehicle, virus or vector is delivered or administered to the individual in need thereof,
wherein optionally the expression vehicle or vector is selected from the group consisting of a herpes simplex virus, a human immunodeficiency virus (HIV), a synthetic vector, an adeno-associated virus (AAV), a lentivirus, an adenovirus and a plasmid,
and optionally the AAV is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant;
   the HMGB2-expressing nucleic acid, or the HMGB2-inhibiting nucleic acid, or the expression construct, plasmid, expression vehicle, virus or vector, or the genetically manipulated or engineered T cell, is formulated:
(a) in a liquid, a gel, a hydrogel, a vesicle, a liposome, a nanoparticle, a nanolipid particle, a powder or an aqueous or a saline formulation, or for administration in vitro or in vivo;
(b) for enteral or parenteral administration;
(c) in or as a liposome, a nanoparticle, or a nanoliposome;
(d) in or as a tablet, a pill, a capsule, a gel, a hydrogel, a geltab, a liquid, a powder, an emulsion, a lotion, an aerosol, a spray, a lozenge, an aqueous or a sterile or an injectable solution, an eye drop, or an implant; or
(e) for intravenous injection, subcutaneous injection, intramuscular injection, inhalation, or intravitreal injection;
   the HMGB2-inhibiting molecule is or comprises a small molecule, an inhibitory nucleic acid (optionally and miRNA or antisense molecule), polypeptide or peptide (optionally an antibody capable of specifically binding to HMGB2 protein and inhibiting its expression or activity), a lipid or a polysaccharide; and/or
   the HMGB2-inhibiting nucleic acid is or comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), an inhibitory ribozyme, or a CRISPR system such as CRISPR-Cas9.

In alternative embodiments, provided are HMGB2-expressing nucleic acids, or an expression construct, plasmid, expression vehicle, virus or vector having contained therein an HMGB2-expressing nucleic acid, or a T cell genetically manipulated or engineered to express or overexpress an HMGB2 polypeptide, or a liposome, a nanoparticle, or a nanoliposome having contained therein an HMGB2-expressing nucleic acid, for use in:
increasing the rate or amount of differentiation and stemness of memory and exhausted CD8⁺ T cells in vivo,
increasing the rate or amounts of effector and memory CD8⁺ T cells in vivo;
expanding a subset of progenitor stem cells within an exhausted T cell population in vivo or ex vivo;
boosting or increasing CD8⁺ T cells during acute viral infection in an individual in need thereof,
increasing memory T cells and memory T cells differentiation, memory precursor effector (MPEC) and central memory (Tcm) T cell phenotypes, and memory recall responses in vivo,
increasing the ability of an individual in need thereof to recover from a viral infection, and/or
treating, ameliorating, slowing the progression of, decreasing the severity of symptoms of, an infection, optionally, a viral infection, in an individual in need thereof.

In alternative embodiments, provided are HMGB2-inhibiting nucleic acids, or an expression construct, plasmid, expression vehicle, virus or vector having contained therein an HMGB2− inhibiting nucleic acid, or a T cell genetically manipulated or engineered to inhibit or decrease expression or activity of an HMGB2 polypeptide, or a liposome, a nanoparticle, or a nanoliposome having contained therein an HMGB2− inhibiting nucleic acid, for use in:
decreasing the rate or amount of differentiation and stemness of memory and exhausted CD8⁺ T cells in vivo,
decreasing the rate or amounts of effector and memory CD8⁺ T cells in vivo;
expanding a subset of progenitor stem cells within an exhausted T cell population in vivo or ex vivo; and/or
treating, ameliorating, slowing the progression of, decreasing the severity of symptoms of, an autoimmune disease, in an individual in need thereof.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-D illustrate HMGB2 expression in murine and human virus-specific CD8⁺ T cells:

FIG. 1A-B graphically illustrates expression levels of HMGB2 in GP₃₃₋₄₁⁺ CD8⁺ T cells (FIG. 1A) and GP₂₇₆₋₂₈₆⁺ CD8⁺ T cells (FIG. 1), right-hand graphs are data assessed by flow cytometry, in spleen with n=4 mice;

FIG. 1C illustrates representative IMAGESTREAM™ analysis of GP₃₃₋₄₁⁺ CD8⁺ T cells, magnification, 60×; and

FIG. 1D-E graphically illustrate low-input RNA-sequencing data from Hensel, N. et al., 2021 (GSE150345)²²: FIG. 1D graphically illustrates average normalized HMGB2 expression in human HCV-specific CD8⁺ T cells, isolated from three patients during chronic Hepatitis C (cHCV) infection and after direct-acting antiviral (DAA) cure; and, FIG. 1E graphically illustrates average FPKM fold change of exhaustion associated genes after cHCV cure in patients from FIG. 1D,
as discussed in further detail in Example 1, below.

FIG. 2A-J illustrate cell-intrinsic kinetics of WT and Hmgb2^{-/-} P14 T cells during Arm infection:

FIG. 2A schematically illustrates an exemplary experimental scheme for FIG. 2B-D, where wild type (WT) and Hmgb2^{-/-} P14 CD8⁺ T cells were transferred separately into naïve mice and infected with lymphocytic choriomeningitis virus (LCMV) arm;

FIG. 2B (left image) graphically illustrates frequency of WT and Hmgb2^{-/-} P14 T cells of total CD8⁺ population from flow cytometry, and FIG. 2B (right image) illustrates the flow cytometry data;

FIG. 2C graphically illustrates splenic WT and Hmgb2^{-/-} P14 T cell frequencies and numbers at 68 dpi, FIG. 2C (left image) graphically illustrates CD8⁺ cell frequencies, FIG. 2C (middle image) graphically illustrates total number of cells, and FIG. 2C (right image) illustrates the flow cytometry data;

FIG. 2D (left image) graphically illustrates cytokine production (TNF gamma and TNF alpha) by splenic WT and Hmgb2^{-/-} P14 T cells at 68 dpi and FIG. 2D (right image) illustrates the flow cytometry data;

FIG. 2E schematically illustrates an exemplary experimental scheme for FIG. 2F-J, where WT and Hmgb2^{-/-} P14 T cells were co-transferred at 1:1 into WT mice and infected with LCMV Arm:

FIG. 2F-G graphically illustrate frequencies (FIG. 2F) and numbers (FIG. 2G) of splenic WT and Hmgb2^{-/-} P14 T cells at indicated timepoints post infection;

FIG. 2H-I graphically illustrate frequencies of splenic WT and Hmgb2^{-/-} P14 short-lived effector (SLEC) and memory precursor effector (MPEC) T cells in the blood at 8 dpi (FIG. 2H) and 46 dpi (FIG. 2I) Arm; and

FIG. 2J graphically illustrates frequencies of WT and Hmgb2^{-/-} P14 central memory (Tcm), effector memory (Tem) and terminal Tem (t-Tem) T cells at 46 dpi Arm in the spleen,
as discussed in further detail in Example 1, below.

FIG. 3A-K illustrate cell-intrinsic kinetics of WT and Hmgb2^{-/-} P14 T cells during C113 infection:

FIG. 3A schematically illustrates an exemplary experimental scheme for FIG. 3B-D;

FIG. 3B-D graphically illustrates data from experiments where WT and Hmgb2^{-/-} P14 CD8⁺ T cells were transferred separately into naïve mice and infected with LCMV C113, and blood was taken at 8, 15, 26, and 35 dpi, and spleens isolated at 68 dpi., FIG. 3B illustrates frequency of WT and Hmgb2^{-/-} P14 T cells of total CD8⁺ population, FIG. 3C illustrates splenic WT and Hmgb2^{-/-} P14 T cell frequencies and numbers at 68 dpi C113, FIG. 3D illustrates cytokine production by splenic WT and Hmgb2^{-/-} P14 T cells at 68 dpi C113;

FIG. 3A schematically illustrates an exemplary experimental scheme for FIG. 3F-G;

FIG. 3F-G graphically illustrate data from experiments where WT and Hmgb2^{-/-} P14 T cells were co-transferred at 1:1 into WT mice and infected with C113, and frequencies (FIG. 3F) and numbers (FIG. 3G) of splenic WT and Hmgb2^{-/-} P14 T cells at indicated timepoints post infection;

FIG. 3H graphically illustrates frequencies of total CD8⁺, GP₃₃₋₄₁⁺, GP₂₇₆₋₂₈₆⁺, and NP₃₉₆₋₄₀₄⁺ T cells during C113 infection in the blood of WT and Hmgb2^{-/-} mice;

FIG. 3I graphically illustrates frequencies of total CD8⁺, GP₃₃₋₄₁⁺, GP₂₇₆₋₂₈₆⁺, and NP₃₉₆₋₄₀₄⁺ T cells during C113 infection in the spleen and lymph nodes (LNs) of WT and Hmgb2^{-/-} mice at 44 dpi; and

FIG. 3J-K graphically illustrate virus titers at 44 dpi measured from sera (FIG. 3J) and kidneys (FIG. 3K) by plaque forming units (PFU) and expressed as PFU/mL or PFU/g, respectively,
as discussed in further detail in Example 1, below.

FIG. 4A-F illustrate that Hmgb2^{-/-} CD8⁺ T cells have decreased expression of Tpex signature genes, data is from studies using bulk RNA-seq analysis of WT and Hmgb2^{-/-} P14 T cells during LCMV Arm and C113 infection:

FIG. 4A graphically illustrates a principal component analysis (PCA) of WT and Hmgb2^{-/-} P14 T cells at 8 days post either Arm or C113 infection;

FIG. 4B graphically illustrates a Volcano plot highlighting differentially expressed genes (DEG) between WT and Hmgb2^{-/-} P14 T cells at 8 dpi C113, and significant DEG (padj≤0.1, |log₂FC|≥0.5) are colored (pink=upregulated in Hmgb2^{-/-} P14 T cells; black=upregulated in WT P14 T cells);

FIG. 4C left image illustrates gene ontology (GO) biological process enrichment from metascape of significant differentially expressed genes (DEG) from FIG. 4B, where the X-axis represents log₁₀(q-value) and size of dot represents proportion of the total DEG enriched to that given pathway, and FIG. 4C right image illustrates a heatmap of average normalized expression of genes associated with bolded pathways, and each column represents one independent experiment with n=5 mice;

FIG. 4D graphically illustrates PCA of co-transferred WT and Hmgb2^{-/-} P14 T cells at 20 dpi C113;

FIG. 4E graphically illustrates a Volcano plot highlighting differentially expressed genes (DEG) between WT and Hmgb2^{-/-} P14 T cells at 20 dpi C113, where significant DEG (padj≤0.1, |log₂FC|≥0.5) are colored (pink=upregulated in Hmgb2^{-/-} P14 T cells; black=upregulated in WT P14 T cells); and

FIG. 4F left image illustrates gene ontology (GO) biological process enrichment from metascape of DEG from FIG. 4E, where X-axis represents log₁₀(q-value) and size of dot represents proportion of the total DEG enriched to that given pathway; FIG. 4F right image illustrates a heatmap of average normalized expression of genes associated with bolded pathways, and each column represents one independent experiment with n=10 mice,
as discussed in further detail in Example 1, below.

FIG. 5A-G illustrate data showing that Hmgb2^{-/-} CD8⁺ T cells have decreased progenitor exhausted T cell differentiation, where WT and Hmgb2^{-/-} P14 T cells were co-transferred at 1:1 into WT mice, followed by LCMV Arm or C113 infection:

FIG. 5A graphically illustrates BrdU uptake of splenic WT and Hmgb2^{-/-} P14 T cells at 8 dpi, with caspase3;

FIG. 5B-C graphically illustrate PI staining of P14 T cells at 8 dpi (FIG. 5B) and 46 dpi (FIG. 5C) in the spleen;

FIG. 5D illustrates images from an alkaline comet assay of splenic WT and Hmgb2^{-/-} P14 T cells isolated on 8 days post either Arm or C113 infection (pooled samples from 10 mice/group), with representative fluorescent comet images of cells stained with Vista Green DNA dye;

FIG. 5E illustrates images of p-H2AX (Ser139) protein expression by Western blot in purified splenic WT and Hmgb2^{-/-} P14 T cells isolated on 8 days post either Arm or C113 infection (pooled samples from 10 mice/group);

FIG. 5F graphically illustrates frequencies and numbers of splenic progenitor exhausted (Tpex) and terminal exhausted (Tex) T cells at 8 dpi C113; and

FIG. 5G illustrates frequencies of CXCR5⁺ P14 T cells at 8 dpi C113 in the spleen,
as discussed in further detail in Example 1, below.

FIG. 6A-J illustrate data showing that memory and exhausted Hmgb2^{-/-} CD8⁺ T cells are defective in their recall capacity:

FIG. 6A schematically illustrates an exemplary experimental scheme for FIG. 6B-D, where WT and Hmgb2^{-/-} P14 T cells were co-transferred into WT mice at 1:1, followed by LCMV Arm infection;

FIG. 6B graphically illustrates the frequency of WT and Hmgb2^{-/-} P14 T cells in blood during secondary Arm infection;

FIG. 6C-D graphically illustrate frequency (FIG. 6C) and number (FIG. 6D) of splenic WT and Hmgb2^{-/-} P14 T cells at 20 dpi secondary Arm;

FIG. 6E graphically illustrates WT and Hmgb2^{-/-} P14 T cells were transferred separately into WT hosts and sorted at 68 dpi Arm before adoptive transfer into separate naïve mice, followed by Arm infection (secondary infection);

FIG. 6F schematically illustrates an exemplary experimental scheme for FIG. 6G-I, where WT and Hmgb2^{-/-} P14 T cells were co-transferred into WT mice at 1:1, followed by LCMV C113 infection;

FIG. 6G graphically illustrates frequency of WT and Hmgb2^{-/-} P14 T cells in blood during secondary Arm infection;

FIG. 6H-I graphically illustrate frequency (FIG. 6H) and number (FIG. 6I) of splenic WT and Hmgb2^{-/-} P14 T cells at 20 dpi secondary Arm; and

FIG. 6J graphically illustrates WT and Hmgb2^{-/-} P14 T cells transferred separately into WT hosts and sorted at 68 dpi Arm before adoptive transfer into separate naïve mice, followed by Arm infection (secondary infection), showing frequency of WT and Hmgb2^{-/-} P14 T cells in the blood during secondary Arm infection,
as discussed in further detail in Example 1, below.

FIG. 7A-G illustrate data showing epigenetic programming of exhausted T cells by HMGB2, where ATAC-seq analysis of WT and Hmgb2^{-/-} P14 T cells at 8 dpi LCMV C113:

FIG. 7A graphically illustrates a principal component analysis (PCA) of all samples by global chromatin accessibility profile;

FIG. 7B graphically illustrates locations of significantly differentially accessible ATAC-seq peaks (FDR≤0.05, |log₁₀FC|≥0.3);

FIG. 7C graphically illustrates a heatmap of all significantly differentially accessible loci (DAR), where numbers on left denote number of DAR, and each column represents a biological replicate of n=10 mice pooled;

FIG. 7D graphically illustrates ATAC-seq tracks of genes associated with effector (Teff) and terminal exhausted (Tex) T cells, where DAR are highlighted with grey bars;

FIG. 7E graphically illustrates a heatmap of DAR within promoters-TSS (≤1 kb);

FIG. 7F graphically illustrates gene ontology (GO) biological process enrichment from METASCAPE™ of DAR within promoters-TSS (≤1 kb) from FIG. 7E; and

FIG. 7G graphically illustrates a fold change in ATAC accessibility versus RNA expression, where key genes with DAR in promoters-TSS (≤1 kb) are highlighted in red,
as discussed in further detail in Example 1, below.

FIG. 8A-D illustrate data showing HMGB2 regulation of anti-tumor CD8⁺ T cells:

FIG. 8A schematically illustrates an exemplary experimental scheme for FIG. 8B-D, where WT and Hmgb2^{-/-} P14 T cells were co-transferred into WT mice at 1:1 and given B16-GP₃₃₋₄₁ melanoma cells subcutaneously (s.c.), and tumors and tumor draining lymph nodes (TdLN) isolated at 18 dpi.;

FIG. 8B-C graphically illustrate frequencies (FIG. 8B) and numbers (FIG. 8C) of WT and Hmgb2^{-/-} P14 T cells within the tumor and TdLN at 18 dpi; and

FIG. 8D graphically illustrates frequencies of progenitor exhausted (Tpex) and terminal exhausted (Tex) T cells at 18 dpi in the tumor,
as discussed in further detail in Example 1, below.

FIG. 9 illustrates images of an exemplary gating strategy used in co-adoptive transfer experiments, including: leukocytes; CD8+ cells; CD8+ and CD45.2+ P14 cells; and, WT and Hmgb2^{-/-} P14 cells, as discussed in further detail in Example 1, below.

FIG. 10A-B graphically illustrate effector function and survival of Hmgb2^{-/-} CD8⁺ T cells, where frequencies of GranzymeB⁺ WT and Hmgb2^{-/-} P14 T cells at 8 dpi LCMV Arm (FIG. 10A) frequency of total Caspase3⁺ splenic WT and Hmgb2^{-/-} short-lived effector (SLEC) and memory precursor effector (MPEC) P14 T cells (FIG. 10B) at 46 dpi Arm, as discussed in further detail in Example 1, below.

FIG. 11A-D graphically illustrate that HMGB2 expression is dispensable for naïve CD8⁺ T cell development, where CCR7/CD127 (FIG. 11A) and CD44/CD62L (FIG. 11B) protein expression of naïve WT and Hmgb2^{-/-} P14 T cells isolated from lymph nodes of uninfected mice. CCR7/CD127 (FIG. 11C) and CD44/CD62L (FIG. 11D) protein expression of naïve WT and Hmgb2^{-/-} P14 T cells isolated from spleens of uninfected mice; and

FIG. 11E graphically illustrates the number of naïve WT and Hmgb2^{-/-} CD8⁺ T cells,
as discussed in further detail in Example 1, below.

FIG. 12A-D graphically illustrate effector function and Apoptosis of WT and Hmgb2^{-/-} P14 SLEC/MPEC and Tpex/Tex cells, where WT and Hmgb2^{-/-} P14 T cells were co-transferred into mice followed by C113 infection, and frequencies of GranzymeB⁺ WT and Hmgb2^{-/-} P14 T cells at 8 dpi LCMV C113 (FIG. 12A) Frequencies of PD-1⁺TIM3⁺ T cells (FIG. 12B) Mean fluorescence expression of LAG-3, PD-1, and TIM-3 in CD8⁺ T cells after C113 infection (FIG. 12C), and TOX expression at various times post-infection in WT Hmgb2^{-/-} P14 T cells (FIG. 12D), as discussed in further detail in Example 1, below.

FIG. 13A-B graphically illustrate transcriptomics of WT and Hmgb2^{-/-} CD8⁺ T cells after acute viral infection:

FIG. 13A illustrates a Volcano plot highlighting differentially expressed genes (DEG) between WT and Hmgb2^{-/-} P14 T cells at 8 dpi LCMV Arm infection from FIG. 4A; and

FIG. 13B illustrates a heatmap of average normalized expression of significant DEG,
as discussed in further detail in Example 1, below.

FIG. 14 illustrates HMGB2 regulates TCF-1 transcriptional network in CD8⁺ T cells during C113 infection: TCF-1 causal network identified with Ingenuity Pathway Core Analysis (IPA), as discussed in further detail in Example 1, below.

FIG. 15A-G graphically illustrate survival and_transcription factor expression in exhausted Hmgb2^{-/-} P14 T cells: P14 progenitor exhausted (Tpex) and terminal exhausted (Tex) T cell numbers at 46 dpi C113 (FIG. 15A), and TCF-1 expression in WT and Hmgb2^{-/-} mice during C113 infection (FIG. 15B), and TCF-1 expression in CD8⁺ and Tetramer⁺CD8⁺ T cells in WT or Hmgb2^{-/-} mice infected with C113 (FIG. 15C), and frequency of total Caspase3⁺ splenic WT and Hmgb2^{-/-} Tpex and Tex cells at 8 dpi (FIG. 15D) and 46 dpi (FIG. 15E) C113, and frequencies of HMGB2⁺ and HMGB2⁻ cells within progenitor exhausted (Tpex) and terminal exhausted (Tex) WT P14 T cells isolated from spleens at 8 dpi C113 (FIG. 15F) Frequencies of Tpex and Tex within HMGB2⁺ and HMGB2⁻ WT P14 T cells isolated from spleens at 8 dpi C113 (FIG. 15G), as discussed in further detail in Example 1, below.

FIG. 16A-B graphically illustrate TCF-1 expression in CD8⁺ T cells during Arm infection: frequencies of TCF-1 expression in WT or Hmgb2^{-/-} P14⁺ CD8⁺ T cells after ARM infection (FIG. 16A) and representative FACS plots (FIG. 16B), as discussed in further detail in Example 1, below.

Like reference symbols in the various drawings indicate like elements.

### DETAILED DESCRIPTION

In alternative embodiments, provided are compositions, including products of manufacture and kits, and methods, for increasing HMGB2 (high-mobility group box2) activity in a T cell, for: increasing the rate or amount of differentiation and stemness of memory and exhausted CD8⁺ T cells in vivo, increasing the rate or amounts of effector and memory CD8⁺ T cells in vivo; expanding a subset of progenitor stem cells within an exhausted T cell population in vivo or ex vivo; boosting or increasing CD8⁺ T cells during acute viral infection in an individual in need thereof, increasing memory T cells and memory T cells differentiation, memory precursor effector (MPEC) and central memory (Tcm) T cell phenotypes, and memory recall responses in vivo, increasing the ability of an individual in need thereof to recover from a viral infection, and/or treating, ameliorating, slowing the progression of, decreasing the severity of symptoms of, an infection, optionally, a viral infection, in an individual in need thereof. In alternative embodiments, provided are compositions, including products of manufacture and kits, and methods, for decreasing HMGB2 (high-mobility group box2) activity in a T cell, for: decreasing the rate or amount of differentiation and stemness of memory and exhausted CD8⁺ T cells in vivo, decreasing the rate or amounts of effector and memory CD8⁺ T cells in vivo; expanding a subset of progenitor stem cells within an exhausted T cell population in vivo or ex vivo; and/or treating, ameliorating, slowing the progression of, decreasing the severity of symptoms of, an autoimmune disease, in an individual in need thereof.

In alternative embodiments, HMGB2 expression is modulated to treat cancer in any immunotherapy setting to change the quantity and quality of the responding T cells. In alternative embodiments, HMGB2 is modulated to increase the efficacy of vaccination strategies against any antigen, for example, for a cancer vaccine, a pathogen vaccine, and the like.

Given HMGB2's role in both modulating chromatin architecture and regulating stem cells, along with its high gene expression in CD8⁺ T cells, we investigated the function of HMGB2 in effector, memory, and exhausted CD8⁺ T cells. We found a cell-intrinsic role for HMGB2 in the differentiation and sternness of memory and exhausted CD8⁺ T cells. Effector, memory and exhausted CD8⁺ T cells had high HMGB2 expression that was sustained with persistent antigen. After acute viral infection, we observed a decrease of Hmgb2^{-/-} CD8⁺ memory T cells, with defective central memory T cell (Tcm) formation and recall capacity. In response to chronic viral infection, Hmgb2^{-/-} CD8⁺ T cells showed decreased Tpex differentiation. Even though Hmgb2^{-/-} CD8⁺ T cells expressed both TCF-1 and TOX, these transcription factors were unable to support the differentiation and maintenance Tpex and Tex cells. Mechanistically, HMGB2 regulated Tpex-specific transcriptional programming through increasing chromatin accessibility of Tpex genes, while decreasing accessibility of regions specific for Tex cells during chronic infection. Our findings show a previously unknown role for HMGB2 as an essential regulator of memory and exhausted CD8⁺ T cell differentiation, that protects these cells from a terminal fate.

### Formulations and Pharmaceutical Compositions

In alternative embodiments, provided are pharmaceutical formulations or compositions comprising nucleic acids and polypeptides for practicing methods and uses as provided herein for increasing HMGB2 (high-mobility group box2) activity in a T cell, for treating, ameliorating, slowing the progression of, decreasing the severity of symptoms of, an infection, optionally, a viral infection, in an individual in need thereof. In alternative embodiments, provided are compositions, including products of manufacture and kits, and methods, for decreasing HMGB2 (high-mobility group box2) activity in a T cell, for: treating, ameliorating, slowing the progression of, decreasing the severity of symptoms of, an autoimmune disease, in an individual in need thereof.

In alternative embodiments, pharmaceutical compositions and formulations used to practice methods and uses as provided herein comprise HMGB2-expressing nucleic acids and polypeptides that result in an increase in expression or activity of HMGB2-expressing nucleic acids and polypeptides when administered to an individual in need thereof. In alternative embodiments, pharmaceutical compositions and formulations used to practice methods and uses as provided herein comprise HMGB2-inhibiting nucleic acids and polypeptides that result in a decrease in expression or activity of HMGB2-expressing nucleic acids and polypeptides when administered to an individual in need thereof.

In alternative embodiments, the pharmaceutical compositions used to practice methods and uses as provided herein can be administered intramuscularly (IM), parenterally, topically, orally or by local administration, such as by aerosol or transdermally, or intravitreal injection. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, these compositions used to practice methods and uses as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods or uses as provided herein can be in forms such as tablets, pills, powders, capsules, liquids, gels, syrups, slurries, suspensions, etc., suitable for in vivo, in vitro or ex vivo applications.

In alternative embodiments, formulations and pharmaceutical compositions used to practice methods and uses as provided herein can comprise a solution of compositions (which include peptidomimetics, racemic mixtures or racemates, isomers, stereoisomers, derivatives and/or analogs of compounds) disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose, any fixed oil can be employed including synthetic mono- or diglycerides, or fatty acids such as oleic acid. In one embodiment, solutions and formulations used to practice methods and uses as provided herein are sterile and can be manufactured to be generally free of undesirable matter. In one embodiment, these solutions and formulations are sterilized by conventional, well known sterilization techniques.

The solutions and formulations used to practice methods and uses as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compositions and formulations used to practice methods and uses as provided herein can be delivered by the use of liposomes. By using liposomes, particularly where the liposome surface carries ligands specific for target cells (for example, a cancer cell), or are otherwise preferentially directed to a specific tissue or organ type, one can focus the delivery of the active agent into a target cells (for example, T cells) in an in vivo, in vitro or ex vivo application.

### Nanoparticles, Nanolipoparticles and Liposomes

Also provided are nanoparticles, nanolipoparticles, vesicles and liposomal membranes comprising compounds used to practice methods and uses as provided herein, for example, to deliver compositions comprising HMGB2-inhibiting or HMGB2-expressing nucleic acids and HMGB2 polypeptides in vivo, for example, to a solid tumor, lymphoid tissue, or for intramuscular injection. In alternative embodiments, these compositions are designed to target specific molecules, including biologic molecules, such as polypeptides, including cell surface polypeptides, for example, for targeting a desired cell type or organ, for example, a cancer cell or lymphoid tissue.

Provided are multilayered liposomes comprising compounds used to practice methods and uses as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods and uses as provided herein.

Liposomes can be made using any method, for example, as described in Park, et al., U.S. Pat. Pub. No. 20070042031, including method of producing a liposome by encapsulating an active agent (for example, XCL1-expressing nucleic acids), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods and uses as provided herein comprise a substituted ammonium and/or polyanions, for example, for targeting delivery of a compound (for example, XCL1-expressing nucleic acid) to a desired cell type (for example, tumor, or lymphoid tissue), as described for example, in U.S. Pat. Pub. No. 20070110798.

Provided are nanoparticles comprising compounds (for example, XCL1-expressing nucleic acid used to practice methods provided herein) in the form of active agent-containing nanoparticles (for example, a secondary nanoparticle), as described, for example, in U.S. Pat. Pub. No. 20070077286. In one embodiment, provided are nanoparticles comprising a fat-soluble active agent or a fat-solubilized water-soluble active agent to act with a bivalent or trivalent metal salt.

In one embodiment, solid lipid suspensions can be used to formulate and to deliver compositions used to practice methods and uses as provided herein to mammalian cells in vivo, for example, to the CNS, as described, for example, in U.S. Pat. Pub. No. 20050136121.

### Delivery Vehicle Modifications and Modification of HMGB2-Expressing Nucleic Acid

In alternative embodiments, HMGB2-inhibiting or HMGB2-expressing nucleic acid, or HMGB2-expressing nucleic acid-comprising nanoparticles, liposomes and the like (for example, comprising or having contained therein HMGB2-expressing nucleic acid used to practice methods provided herein) are modified to facilitate IV, IM or any in vivo injections. For example, in alternative embodiments, HMGB2-inhibiting or HMGB2-expressing nucleic acid-comprising nanoparticles, liposomes and the like, are engineered to comprise a moiety that allows the HMGB2-inhibiting or HMGB2-expressing nucleic acid-comprising nanoparticles, liposomes and the like, to bind to a receptor or cell membrane structure that facilitates delivery into or to a desired cell or organ or tissue, for example to a T cell.

### Delivery Cells and Delivery Vehicles

In alternative embodiments, any delivery vehicle can be used to practice the methods or uses as provided herein, for example, to deliver compositions (for example, HMGB2-inhibiting or HMGB2-expressing nucleic acids) in vivo. For example, delivery vehicles comprising polycations, cationic polymers and/or cationic peptides, such as polyethyleneimine derivatives, can be used for example as described, for example, in U.S. Pat. Pub. No. 20060083737. In one embodiment, a delivery vehicle is a transduced cell engineered to express or overexpress and then secrete an endogenous or exogenous HMGB2-inhibiting or HMGB2-expressing nucleic acid.

In one embodiment, a dried polypeptide-surfactant complex is used to formulate a composition used to practice methods as provided herein, for example as described, for example, in U.S. Pat. Pub. No. 20040151766.

In one embodiment, a composition used to practice methods and uses as provided herein can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, cells that will be subsequently delivered into a tumor or lymphoid tissue are transfected or transduced with HMGB2-inhibiting or HMGB2-expressing nucleic acids, for example, a vector, for example, by electro-permeabilization, which can be used as a primary or adjunctive means to deliver the composition to a cell, for example, using any electroporation system as described for example in U.S. Pat. Nos. 7,109,034; 6,261,815; 5,874,268.

In alternative embodiments, HMGB2-inhibiting or HMGB2-expressing nucleic acids used to practice embodiments as provided herein comprise or are comprised of, or are designed based on: human cDNA sequence (SEQ ID NO:1):

>ENST00000446922.6 HMGB2-203 cdna: protein_coding
GCAAAACAAGAGGCTGAGGATTGCGTTAGAGATAAACCAGTTCACGCCGGAGCCCCGTGA
GGGAAGCGTCTCCGTTGGGTCCGGCCGCTCTGCGGGACTCTGAGGAAAAGCTCGCACCAG
GCAAGAATACCCTCCAATACCCTCGGGTGGACGCGGATCTGTCAACATGGGTAAAGGAGA
CCCCAACAAGCCGCGGGGCAAAATGTCCTCGTACGCCTTCTTCGTGCAGACCTGCCGGGA
AGAGCACAAGAAGAAACACCCGGACTCTTCCGTCAATTTCGCGGAATTCTCCAAGAAGTG
TTCGGAGAGATGGAAGACCATGTCTGCAAAGGAGAAGTCGAAGTTTGAAGATATGGCAAA
AAGTGACAAAGCTCGCTATGACAGGGAGATGAAAAATTACGTTCCTCCCAAAGGTGATAA
GAAGGGGAAGAAAAAGGACCCCAATGCTCCTAAAAGGCCACCATCTGCCTTCTTCCTGTT
TTGCTCTGAACATCGCCCAAAGATCAAAAGTGAACACCCTGGCCTATCCATTGGGGATAC
TGCAAAGAAATTGGGTGAAATGTGGTCTGAGCAGTCAGCCAAAGATAAACAACCATATGA
ACAGAAAGCAGCTAAGCTAAAGGAGAAATATGAAAAGGATATTGCTGCATATCGTGCCAA
GGGCAAAAGTGAAGCAGGAAAGAAGGGCCCTGGCAGGCCAACAGGCTCAAAGAAGAAGAA
CGAACCAGAAGATGAGGAGGAGGAGGAGGAAGAAGAAGATGAAGATGAGGAGGAAGAGGA
TGAAGATGAAGAATAAATGGCTATCCTTTAATGATGCGTGTGGAATGTGTGTGTGTGCTC
AGGCAATTATTTTGCTAAGAATGTGAATTCAAGTGCAGCTCAATACTAGCTTCAGTATAA
AAACTGTACAGATTTTTGTATAGCTGATAAGATTCTCTGTAGAGAAAATACTTTTAAAAA
ATGCAGGTTGTAGCTTTTTGATGGGCTACTCATACAGTTAGATTTTACAGCTTCTGATGT
TGAATGTTCCTAAATATTTAATGGTTTTTTTAATTTCTTGTGTATGGTAGCACAGCAAAC
TTGTAGGAATTAGTATCAATAGTAAATTTTGGGTTTTTTAGGATGTTGCATTTCGTTTTT
TTAAAAAAAATTTTGTAATAAAATTATGTATATTA

or a nucleic acid having between about 90% to 99% sequence identity to SEQ ID NO:1,
and optionally the HMGB2-inhibiting or HMGB2-expressing nucleic acid comprises, or has a design based on, a nucleic acid sequence that encodes the polypeptide:

(SEQ ID NO: 2)
MGKGDPNKPRGKMSSYAFFVQTCREEHKKKHPDSSVNFAEFSKKCSERW
KTMSAKEKSKFEDMAKSDKARYDREMKNYVPPKGDKKGKKKDPNAPKRP
PSAFFLFCSEHRPKIKSEHPGLSIGDTAKKLGEMWSEQSAKDKQPYEQK
AAKLKEKYEKDIAAYRAKGKSEAGKKGPGRPTGSKKKNEPEDEEEEEEE
EDEDEEEEDEDEE

or a polypeptide having between about 90% to 99% sequence identity to SEQ ID NO:2, or a polypeptide having about 92%, 95% or 97% sequence identity to SEQ ID NO:2,
wherein optionally the sequence identities (SEQ IDs) are determined by analysis with a sequence comparison algorithm or by a visual inspection, and optionally the sequence comparison algorithm is a BLAST version 2.2.2 algorithm where a filtering setting is set to blastall -p blastp -d “nr pataa”-F F, and all other options are set to default.

Various sequence comparison programs identified in this patent specification are particularly contemplated for use in this embodiment. Protein and/or nucleic acid sequence homologies may be evaluated using any of the variety of sequence comparison algorithms and programs known in the art. Such algorithms and programs include, but are by no means limited to, TBLASTN, BLASTP, FASTA, TFASTA and CLUSTALW (see, e.g., Pearson and Lipman, Proc. Natl. Acad. Sci. USA 85(8):2444-2448, 1988; Altschul et al., J. Mol. Biol. 215(3):403-410, 1990; Thompson Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol 48:443, 1970, by the search for similarity method of person & Lipman, Proc. Nat'l. Acad. Sci. USA 85:2444, 1988, by computerized implementations of these algorithms (GAP™, BESTFIT™, FASTA™ and TFASTA™ in the Wisconsin Genetics Software Package, Genetics Computer Group, 575 Science Dr., Madison, WI), or by manual alignment and visual inspection. Other algorithms for determining homology or identity include, for example, in addition to a BLAST program (Basic Local Alignment Search Tool at the National Center for Biological Information), ALIGN™, AMAS™ (Analysis of Multiply Aligned Sequences), AMPS™ (Protein Multiple Sequence Alignment), ASSET™ (Aligned Segment Statistical Evaluation Tool), BANDS™, BESTSCOR™, BIOSCAN™ (Biological Sequence Comparative Analysis Node), BLIMIPS™ (BLocks IMProved Searcher), FASTA™, Intervals & Points, BMB™, CLUSTAL V™, CLUSTAL W™ CONSENSUS™, LCONSENSUS, WCONSENSUS™, Smith-Waterman algorithm, DARWIN™, Las Vegas algorithm, FNAT (Forced Nucleotide Alignment Tool), FRAMEALIGN™, FRAMESEARCH™, DYNAMIC™, FILTER™, FSAP™ (Fristensky Sequence Analysis Package), GAP™ (Global Alignment Program), GENAL™, GIBBS™, GENQUEST™, ISSC™ (Sensitive Sequence Comparison), LALIGN™ (Local Sequence Alignment), LCP™ (Local Content Program), MACAW™ (Multiple Alignment Construction & Analysis Workbench), MAP™ (Multiple Alignment Program), MBLKP™, MBLKN™, PIMA™ (Pattern-Induced Multi-sequence Alignment), SAGA™ (Sequence Alignment by Genetic Algorithm) and WHAT-IF™. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). At least twenty-one other genomes have already been sequenced, including, for example, M. genitalium (Fraser et al., 1995), M. jannaschii (Bult et al., 1996), H. influenzae (Fleischmann et al., 1995), E. coli (Blattner et al., 1997) and yeast (S. cerevisiae) (Mewes et al., 1997) and D. melanogaster (Adams et al., 2000). Significant progress has also been made in sequencing the genomes of model organism, such as mouse, C. elegans and Arabidopsis sp. Several databases containing genomic information annotated with some functional information are maintained by different organizations and may be accessible via the internet.

One example of a useful algorithm is BLAST™ and BLAST 2.0™ algorithms, which are described in Altschul et al., Nuc. Acids Res. 25:3389-3402, 1977 and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST™ algorithm parameters W, T and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3 and expectations (E) of 10 and the BLOSUM62 scoring matrix (see Henikoff & Henikoff, Proc. Natl. Acad. Sci. USA 89:10915, 1989) alignments (B) of 50, expectation (E) of 10, M=5, N=−4 and a comparison of both strands.

The BLAST algorithm also performs a statistical analysis of the similarity between two sequences (see, e.g., Karlin & Altschul, Proc. Natl. Acad. Sci. USA 90:5873, 1993). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more in one aspect less than about 0.01 and most in one aspect less than about 0.001.

In one aspect, protein and nucleic acid sequence homologies are evaluated using the Basic Local Alignment Search Tool (“BLAST™”) In particular, five specific BLAST™ programs are used to perform the following task:
(1) BLASTP™ and BLAST3™ compare an amino acid query sequence against a protein sequence database;
(2) BLASTN™ compares a nucleotide query sequence against a nucleotide sequence database;
(3) BLASTX™ compares the six-frame conceptual translation products of a query nucleotide sequence (both strands) against a protein sequence database;
(4) TBLASTN™ compares a query protein sequence against a nucleotide sequence database translated in all six reading frames (both strands); and
(5) TBLASTX™ compares the six-frame translations of a nucleotide query sequence against the six-frame translations of a nucleotide sequence database.

The BLAST™ programs identify homologous sequences by identifying similar segments, which are referred to herein as “high-scoring segment pairs,” between a query amino or nucleic acid sequence and a test sequence which is in one aspect obtained from a protein or nucleic acid sequence database. High-scoring segment pairs are in one aspect identified (i.e., aligned) by means of a scoring matrix, many of which are known in the art. In one aspect, the scoring matrix used is the BLOSUM62 matrix (Gonnet (1992) Science 256:1443-1445; Henikoff and Henikoff (1993) Proteins 17:49-61). Less in one aspect, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). BLAST programs are accessible through the U.S. National Library of Medicine.

The parameters used with the above algorithms may be adapted depending on the sequence length and degree of homology studied. In some aspects, the parameters may be the default parameters used by the algorithms in the absence of instructions from the user.

In alternative embodiments, the HMGB2-inhibiting or HMGB2-expressing nucleic acid is or comprises an natural or a synthetic oligonucleotide, and optionally the natural or synthetic oligonucleotide comprises an RNA or a DNA, and optionally the natural or synthetic oligonucleotide comprises a small inhibitory RNA (siRNA), a cDNA, an mRNA, an shRNA, an miRNA or a gRNA, and optionally the deoxyribonucleic acid (DNA) comprises an antisense oligonucleotide, and optionally the antisense oligonucleotide comprises a modified or synthetic anti-sense oligonucleotide (ASO), and optionally the modified or synthetic anti-sense oligonucleotide comprises: a 2′-O-(2-MethoOxyEthyl)-oligoribonucloeotide (2′-MOE); a 2′-O-Methyl-RNA (2′-OMe) oligoribonucloeotide; a phosphorothioate DNA or an oligonucleotide phosphorothioate (OPS); a phosphorodiamidate morpholino oligomer (PMO); a tricyclo-DNA or tcDNA; a locked nucleic acid (LNA) or inaccessible RNA (or RNA nucleotide having a ribose moiety modified with an extra bridge connecting a 2′ oxygen and a 4′ carbon); a morpholino, 5′ methylcytosine base; and/or other modified nucleic acids.

### Antisense Inhibitory Nucleic Acid Molecules

In alternative embodiments, HMGB2-inhibiting pharmaceutical compositions and formulations methods as provided herein are administered to an individual in need thereof in an amount sufficient to practice methods as provided herein.

In alternative embodiments, provided are compositions and methods for administering HMGB2-inhibiting nucleic acids, for example, an antisense morpholino oligonucleotide (MO), an miRNA, an siRNA and the like.

In alternative embodiments, compositions and methods as provided herein comprise use of an inhibitory nucleic acid molecule or an antisense oligonucleotide inhibitory to expression of an HMGB2 protein activity. In alternative embodiments, compositions and methods as provided herein comprise use of an inhibitory nucleic acid molecule or antisense oligonucleotide inhibitory to expression of HMGB2, comprising: an RNAi inhibitory nucleic acid molecule, a double-stranded RNA (dsRNA) molecule, a small interfering RNA (siRNA), a microRNA (miRNA) and/or a short hairpin RNA (shRNA), or a ribozyme.

Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol. Appl. Pharmacol. 144:189-197; Antisense Therapeutics, ed. Agrawal (Humana Press, Totowa, N.J., 1996). Antisense oligonucleotides having synthetic DNA backbone analogues can also include phosphoro-dithioate, methylphosphonate, phosphoramidate, alkyl phosphotriester, sulfamate, 3′-thioacetal, methylene (methylimino), 3′-N-carbamate, and morpholino carbamate nucleic acids.

### RNA Interference (RNAi)

In alternative embodiments, provided are RNAi inhibitory nucleic acid molecules capable of decreasing or inhibiting expression of HMGB2, and including for example, decreasing or inhibiting expression of the transcript (mRNA, message) or isoform or isoforms thereof. In one aspect, the RNAi molecule comprises a double-stranded RNA (dsRNA) molecule. The RNAi molecule can comprise a double-stranded RNA (dsRNA) molecule, e.g., siRNA, miRNA (microRNA) and/or short hairpin RNA (shRNA) molecules.

In alternative aspects, the RNAi is about 11, 12, 13, 14, 15, 16, 17, 18, 19, 20, 21, 22, 23, 24, 25 or more duplex nucleotides in length. While the methods provided herein are not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, e.g., siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence.

In one aspect, intracellular introduction of the RNAi (e.g., miRNA or siRNA) is by internalization of a target cell specific ligand bonded to an RNA binding protein comprising an RNAi (e.g., microRNA) is adsorbed. The ligand can be specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, provided are lipid-based formulations for delivering, e.g., introducing nucleic acids used in methods as provided herein, as nucleic acid-lipid particles comprising an RNAi molecule to a cell, see, e.g., U.S. Patent App. Pub. No. 20060008910.

Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127.

Methods for making expression constructs, e.g., vectors or plasmids, from which an inhibitory polynucleotide (e.g., a duplex siRNA) is transcribed are well known and routine. A regulatory region (e.g., promoter, enhancer, silencer, splice donor, acceptor, etc.) can be used to transcribe an RNA strand or RNA strands of an inhibitory polynucleotide from an expression construct. When making a duplex siRNA inhibitory molecule, the sense and antisense strands of the targeted portion of the targeted IRES can be transcribed as two separate RNA strands that will anneal together, or as a single RNA strand that will form a hairpin loop and anneal with itself. For example, a construct targeting a portion of a gene, e.g., an MMP coding sequence or transcriptional activation sequence, is inserted between two promoters (e.g., mammalian, viral, human, tissue specific, constitutive or other type of promoter) such that transcription occurs bidirectionally and will result in complementary RNA strands that may subsequently anneal to form an inhibitory siRNA used to practice methods as provided herein.

Alternatively, a targeted portion of a gene, coding sequence, promoter or transcript can be designed as a first and second antisense binding region together on a single expression vector; for example, comprising a first coding region of a targeted gene in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene-inhibitory siRNA used to practice methods as provided herein.

In another aspect, transcription of the sense and antisense targeted portion of the targeted gene is controlled by a single promoter, and the resulting transcript will be a single hairpin RNA strand that is self-complementary, i.e., forms a duplex by folding back on itself to create a gene-inhibitory siRNA molecule. In this configuration, a spacer, e.g., of nucleotides, between the sense and antisense coding regions of the targeted portion of the targeted gene can improve the ability of the single strand RNA to form a hairpin loop, wherein the hairpin loop comprises the spacer. In one embodiment, the spacer comprises a length of nucleotides of between about 5 to 50 nucleotides. In one aspect, the sense and antisense coding regions of the siRNA can each be on a separate expression vector and under the control of its own promoter.

### Inhibitory Ribozymes

In alternative embodiment, compositions and methods as provided herein comprise use of ribozymes capable of binding and inhibiting, e.g., decreasing or inhibiting, expression of HMGB2.

These ribozymes can inhibit a gene's activity by, e.g., targeting a genomic DNA or an mRNA (a message, a transcript). Strategies for designing ribozymes and selecting a gene-specific antisense sequence for targeting are well described in the scientific and patent literature, and the skilled artisan can design such ribozymes using these reagents. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

### In Vivo Delivery of HMGB2-Inhibiting or HMGB2-Expressing Nucleic Acids

In alternative embodiments, provided are compositions and methods for delivering HMGB2-inhibiting or HMGB2-expressing, or vectors or recombinant viruses having contained therein these nucleic acids. In alternative embodiments, the nucleic acids, vectors or recombinant viruses are designed for in vivo or T cell delivery and expression.

In alternative embodiments, provided are compositions and methods for the delivery and controlled expression of HMGB2-inhibiting or HMGB2-expressing nucleic acid or gene, or an expression vehicle (for example, vector, recombinant virus, and the like) comprising (having contained therein) an HMGB2-encoding nucleic acid or gene, that results in an HMGB2 protein being released into the bloodstream or general circulation where it can have a beneficial effect on in the body.

In alternative embodiments, the provided are methods for being able to turn on and turn off HMGB2-encoding nucleic acids or gene expression easily and efficiently for tailored treatments and insurance of optimal safety.

In alternative embodiments, HMGB2 protein or proteins expressed by the HMGB2-encoding nucleic acids or gene(s) have a beneficial or favorable effects (for example, therapeutic or prophylactic) on a tissue or an organ, for example, an anti-autoimmune or a T cell stimulating effect, even though secreted into the blood or general circulation at a distance (for example, anatomically remote) from their site or sites of action.

In alternative embodiments, provided are expression vehicles, vectors, recombinant viruses and the like for in vivo expression of HMGB2-inhibiting or HMGB2-encoding nucleic acids or gene to practice the methods as provide herein. In alternative embodiments, the HMGB2-inhibiting or HMGB2-encoding nucleic acids (such as RNA or DNA), expression vehicles, vectors, recombinant viruses and the like expressing the an XCL1-encoding nucleic acids or gene can be delivered by intravitreal injection or intramuscular (IM) injection (using for example, HMGB2-inhibiting or HMGB2-encoding RNA in liposomes), by intravenous (IV) injection, by subcutaneous injection, by inhalation, by a biolistic particle delivery system (for example, a so-called “gene gun”), and the like, for example, as an outpatient, for example, during an office visit.

In alternative embodiments, this “peripheral” mode of delivery, for example, expression vehicles, vectors, recombinant viruses and the like injected intravitreal, IM or IV, can circumvent problems encountered when genes or nucleic acids are expressed directly in an organ (for example, a tumor, a lymphoid organ, the brain or into the CNS) itself. Sustained secretion of HMGB2-inhibiting or HMGB2-encoding nucleic acid in the bloodstream or general circulation also circumvents the difficulties and expense of administering proteins by infusion.

In alternative embodiments a recombinant virus (for example, a long-term virus or viral vector), or a vector, or an expression vector, and the like, can be injected, for example, in a systemic vein (for example, IV), or by intravitreal, intramuscular (IM) injection, by inhalation, or by a biolistic particle delivery system (for example, a so-called “gene gun”), for example, as an outpatient, for example, in a physician's office. In alternative embodiments, days or weeks later (for example, four weeks later), the individual, patient or subject is administered (for example, inhales, is injected or swallows), a chemical or pharmaceutical that induces expression of the HMGB2-inhibiting or HMGB2-encoding nucleic acids or genes; for example, an oral antibiotic (for example, doxycycline or rapamycin) is administered once daily (or more or less often), which will activate the expression of the gene. In alternative embodiments, after the “activation”, or inducement of expression (for example, by an inducible promoter) of the nucleic acid or gene, an HMGB2 protein is synthesized and released into the subject's circulation (for example, into the blood), and subsequently has favorable physiological effects, for example, therapeutic or prophylactic, that benefit the individual or patient (for example, benefit heart, kidney or lung function). When the physician or subject desires discontinuation of the treatment, the subject simply stops taking the activating chemical or pharmaceutical, for example, antibiotic.

Alternative embodiments comprise use of “expression cassettes” comprising or having contained therein a nucleotide sequence used to practice methods provided herein, for example, HMGB2-inhibiting or HMGB2-encoding nucleic acid, which can be capable of affecting expression of the nucleic acid, for example, as a structural gene or a transcript (for example, encoding an HMGB2 protein) in a host compatible with such sequences. Expression cassettes can include at least a promoter operably linked with the polypeptide coding sequence or inhibitory sequence; and, in one aspect, with other sequences, for example, transcription termination signals. Additional factors necessary or helpful in effecting expression may also be used, for example, enhancers.

In alternative aspects, expression cassettes also include plasmids, expression vectors, recombinant viruses, any form of recombinant “naked DNA” vector, and the like. In alternative aspects, a “vector” can comprise a nucleic acid that can infect, transfect, transiently or permanently transduce a cell. In alternative aspects, a vector can be a naked nucleic acid, or a nucleic acid complexed with protein or lipid. In alternative aspects, vectors can comprise viral or bacterial nucleic acids and/or proteins, and/or membranes (for example, a cell membrane, a viral lipid envelope, etc.). In alternative aspects, vectors can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, a vector can be stably replicated by the cells during mitosis as an autonomous structure, or can be incorporated within the host's genome.

In alternative aspects, “promoters” include all sequences capable of driving transcription of a coding sequence in a cell, for example, a mammalian cell such as a retinal cell. Promoters used in the constructs provided herein include cis-acting transcriptional control elements and regulatory sequences that are involved in regulating or modulating the timing and/or rate of transcription of a nucleic acid, for example, HMGB2-inhibiting or HMGB2-encoding nucleic acid. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

In alternative embodiments, “constitutive” promoters can be those that drive expression continuously under most environmental conditions and states of development or cell differentiation. In alternative embodiments, “inducible” or “regulatable” promoters can direct expression of a nucleic acid, for example, an AIBP-encoding nucleic acid, under the influence of environmental conditions, administered chemical agents, or developmental conditions.

### Gene Therapy and Gene Delivery Vehicles

In alternative embodiments, methods as provided herein comprise use of nucleic acid (for example, HMGB2-inhibiting or HMGB2-encoding nucleic acid) delivery systems to deliver a payload of the nucleic acid or gene, or XCL1-encoding nucleic acid, transcript or message, to a cell or cells in vitro, ex vivo, or in vivo, for example, as gene therapy delivery vehicles.

In alternative embodiments, expression vehicle, vector, recombinant virus, or equivalents used to practice methods provided herein are or comprise: an adeno-associated virus (AAV), a lentiviral vector or an adenovirus vector; an AAV serotype AAV5, AAV6, AAV8 or AAV9; a rhesus-derived AAV, or the rhesus-derived AAV AAVrh.10hCLN2; an organ-tropic AAV, or a neurotropic AAV; and/or an AAV capsid mutant or AAV hybrid serotype. In alternative embodiments, the AAV is engineered to increase efficiency in targeting a specific cell type that is non-permissive to a wild type (wt) AAV and/or to improve efficacy in infecting only a cell type of interest. In alternative embodiments, the hybrid AAV is retargeted or engineered as a hybrid serotype by one or more modifications comprising: 1) a transcapsidation, 2) adsorption of a bi-specific antibody to a capsid surface, 3) engineering a mosaic capsid, and/or 4) engineering a chimeric capsid. It is well known in the art how to engineer an adeno-associated virus (AAV) capsid in order to increase efficiency in targeting specific cell types that are non-permissive to wild type (wt) viruses and to improve efficacy in infecting only the cell type of interest; see for example, Wu et al., Mol. Ther. 2006 September; 14(3):316-27. Epub 2006 Jul. 7; Choi, et al., Curr. Gene Ther. 2005 June; 5(3):299-310.

For example, in alternative embodiments, serotypes AAV-8, AAV-9, AAV-DJ or AAV-DJ/8™ (Cell Biolabs, Inc., San Diego, CA), which have increased uptake in brain tissue in vivo, are used to deliver a nucleic acid payload for expression in the CNS. In alternative embodiments, the following serotypes, or variants thereof, are used for targeting a specific tissue:

| Tissue | Optimal Serotype |
|---|---|
| CNS | AAV1, AAV2, AAV4, AAV5, AAV8, AAV9 |
| Photoreceptor Cells | AAV2, AAV5, AAV8 |
| RPE (Retinal Pigment | AAV1, AAV2, AAV4, AAV5, AAV8 |
| Epithelium) | |
| Skeletal Muscle | AAV1, AAV6, AAV7, AAV8, AAV9 |

In alternative embodiments, the rhesus-derived AAV AAVrh.10hCLN2 or equivalents thereof can be used, wherein the rhesus-derived AAV may not be inhibited by any pre-existing immunity in a human; see for example, Sondhi, et al., Hum Gene Ther. Methods. 2012 October; 23(5):324-35, Epub 2012 Nov. 6; Sondhi, et al., Hum Gene Ther. Methods. 2012 Oct. 17; teaching that direct administration of AAVrh.10hCLN2 to the CNS of rats and non-human primates at doses scalable to humans has an acceptable safety profile and mediates significant payload expression in the CNS.

Because adeno-associated viruses (AAVs) are common infective agents of primates, and as such, healthy primates carry a large pool of AAV-specific neutralizing antibodies (NAbs) which inhibit AAV-mediated gene transfer therapeutic strategies, methods provided herein can comprise screening of patient candidates for AAV-specific NAbs prior to treatment, especially with the frequently used AAV8 capsid component, to facilitate individualized treatment design and enhance therapeutic efficacy; see, for example, Sun, et al., J. Immunol. Methods. 2013 Jan. 31; 387(1-2):114-20, Epub 2012 Oct. 11.

In alternative embodiments, the HMGB2-inhibiting or HMGB2-encoding nucleic acid is delivered in vivo using methods as provided herein can be in the form of, or comprise, an RNA, for example, or miRNA or an mRNA, which can be formulated in a lipid formulation or a liposome and injected for example intramuscularly (IM), for example using formulations and methods as described in U.S. patent application no. US 20210046173 A1, which describes delivering to a subject (for example, via intramuscular administration) the HMGB2-inhibiting or HMGB2-encoding nucleic acid that comprises a RNA (for example, miRNA or mRNA) that comprises an open reading frame (ORF) that comprises (or consists of, or consists essentially of) or encodes for the protein; wherein optionally the RNA (or the DNA-carrying expression vehicle) is formulated in a liposome, or a lipid nanoparticle (LNP), or nanoliposome, that comprises: non-cationic lipids comprise a mixture of cholesterol and 1,2-distearoyl-sn-glycero-3-phosphocholine (DSPC), or a PEG-lipid, or PEG-modified lipid, or LNP, or an ionizable cationic lipid; or a mixture of (13Z,16Z)—N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9 Z)-octadec-9-en-1-yloxy]propan-2-amine; (13Z,16Z)—N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy) heptadecanedioate (L319), (13Z,16Z)—N,N-dimethyl-3-nonyldocosa-13,16-dien-1-amine, (12Z,15Z)—N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)—N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

### CRISPR Gene Therapy

In alternative embodiments, nucleic acids (for example, HMGB2-expressing or HMGB2-inhibitory exogenous nucleic acids, including DNA and RNA) are delivered in vivo or ex vivo or nucleic acids are altered in vivo or ex vivo using a CRISPR system such as CRISPR-Cas9. In alternative embodiments, nucleic acids (for example, HMGB2-expressing or HMGB2-inhibitory exogenous nucleic acids, including DNA and RNA) are delivered in vivo or ex vivo to T cells using a CRISPR system such as CRISPR-Cas9.

Delivery of HMGB2-expressing or HMGB2-inhibitory exogenous nucleic acids, Cas9, sgRNA, and associated complexes into cells such as T cells can occur using viral and non-viral systems: for example, electroporation of DNA, RNA, or ribonucleocomplexes; chemical transfection techniques utilizing lipids and peptides (particularly to introduce sgRNAs in complex with Cas9 into cells); nanoparticle-based delivery for transfection, and the like. Some categories of cells are more difficult to transfect, including stem cells, neurons, and hematopoietic cells, and these require more efficient delivery systems, such as those based on lentivirus (LVs), adenovirus (AdV), and adeno-associated virus (AAV).

Variants of CRISPR-Cas9 an be used to allow gene activation or genome editing with an external trigger such as light or small molecules: including photoactivatable CRISPR systems developed by fusing light-responsive protein partners with an activator domain and a dCas9 for gene activation, or by fusing similar light-responsive domains with two constructs of split-Cas9, or by incorporating caged unnatural amino acids into Cas9, or by modifying the guide RNAs with photocleavable complements for genome editing.

In alternative embodiments, “dead” versions of Cas9 (dCas9) are used to eliminate CRISPR's DNA-cutting ability while preserving its ability to target desirable sequences. Various regulatory factors can be added to dCas9s, enabling turning any gene on or off or adjust its level of activity. Like RNAi, CRISPR interference (CRISPRi) can turn off genes in a reversible fashion by targeting, but not cutting a site. The targeted site is methylated, epigenetically modifying the gene. This modification inhibits transcription. These precisely placed modifications may then be used to regulate the effects on gene expression (for example, HMGB2) and DNA dynamics after the inhibition of certain genome sequences within DNA.

In alternative embodiments, CRISPR-Cas13 fused to deaminases is used to direct mRNA editing; for example, Cas7-11, is better suited for therapeutic RNA editing than Cas13, and enables sufficiently targeted cuts.

In alternative embodiments, any CRISPR system can be used to practice methods as provided herein, for example, as described in US 2022 0387560 A1, which describes methods of treating and/or correcting ocular disease in vivo using an Adeno-associated virus (AAV) system, where the AAV system employs a nucleic acid encoding a CRISPR-Cas9 system for targeted gene disruption or correction; or US 2022 0389398 A1 that describes using engineered CRISPR/Cas effector enzymes, such as Cas13 (Cas13d, Cas13e, or Cas13f) that maintain guide-sequence-specific endonuclease activity and lack guide-sequence-independent collateral endonuclease activity; or US 2023 0029506 A1, which describes therapeutic applications of the crispr-cas systems and compositions for genome editing; or, US 2020 0340012 A1, which describes a modular CRISPR-Cas9 architecture that allows better delivery, specificity and selectivity of gene editing; or U.S. Pat. No. 8,771,945, which describes CRISPR-Cas systems and methods for altering expression of gene products; or WO 2023 283420 A2 which describes therapeutic gene silencing with crispr-cas13.

### Dosaging

In alternative embodiments, provided are pharmaceutical formulations or compositions comprising nucleic acids and polypeptides for practicing methods and uses as provided herein for increasing HMGB2 (high-mobility group box2) activity in a T cell, for treating, ameliorating, slowing the progression of, decreasing the severity of symptoms of, an infection, optionally, a viral infection, in an individual in need thereof. In alternative embodiments, provided are compositions, including products of manufacture and kits, and methods, for decreasing HMGB2 (high-mobility group box2) activity in a T cell, for: treating, ameliorating, slowing the progression of, decreasing the severity of symptoms of, an autoimmune disease, in an individual in need thereof. The amount of pharmaceutical composition adequate to accomplish these defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

In alternative embodiments, viral vectors such as adenovirus or AAV vectors are administered to an individual in need therein, and in alternative embodiment the dosage administered to a human comprises: a dose of about 2×10¹² vector genomes per kg body weight (vg/kg), or between about 10¹⁰ and 10¹⁴ vector genomes per kg body weight (vg/kg), or about 10⁹, 10¹⁰, 10¹¹, 10¹², 10¹³, 10¹⁴, 10¹⁵, or more vg/kg, which can be administered as a single dosage or in multiple dosages, as needed. In alternative embodiments, these dosages are administered intravitreally, orally, IM, IV, or intrathecally. In alternative embodiments, the vectors are delivered as formulations or pharmaceutical preparations, for example, where the vectors are contained in a nanoparticle, a particle, a micelle or a liposome or lipoplex, a polymersome, a polyplex or a dendrimer.

In alternative embodiments, these dosages are administered once a day, once a week, or any variation thereof as needed to maintain in vivo expression levels of HMGB2-inhibiting or HMGB2-expressing nucleic acid or HMGB2 protein, which can be monitored by measuring actually expression of XCR1 or by monitoring of therapeutic effect. The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, alternative exemplary pharmaceutical formulations for oral administration of compositions used to practice methods as provided herein are in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods as provided herein can further comprise co-administration with other drugs or pharmaceuticals, for example, compositions for treating any neurological or neuromuscular disease, condition, infection or injury, including related inflammatory and autoimmune diseases and conditions, and the like. For example, the methods and/or compositions and formulations as provided herein can be co-formulated with and/or co-administered with, fluids, antibiotics, cytokines, immunoregulatory agents, anti-inflammatory agents, pain alleviating compounds, complement activating agents, such as peptides or proteins comprising collagen-like domains or fibrinogen-like domains (for example, a ficolin), carbohydrate-binding domains, and the like and combinations thereof.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: HMGB2 Regulates the Differentiation and Stemness of Exhausted CD8+ T Cells During Chronic Viral Infection and Cancer

This example demonstrates that methods and compositions as provided herein using exemplary embodiments are effective and can be used to modulate differentiation and stemness of memory and exhausted CD8⁺ T cells, and that HMGB2 expression can be modulated to treat cancer in any immunotherapy setting to change the quantity and quality of the responding T cells; or that HMGB2 can be modulated to increase the efficacy of vaccination strategies against any antigen, for example, for a cancer vaccine, a pathogen vaccine, and the like.

Chronic infections and cancers evade the host immune system through mechanisms that induce T cell exhaustion. The heterogeneity within the exhausted CD8⁺ T cell pool has revealed the importance of stem-like progenitor (Tpex) and terminal (Tex) exhausted T cells, although the mechanisms underlying their development are not fully known. Here we report HMGB2 expression is upregulated and sustained in exhausted CD8⁺ T cells, and HMGB2 expression is critical for their differentiation. Through epigenetic and transcriptional programming, we identify HMGB2 as a cell-intrinsic regulator of the differentiation and maintenance of Tpex cells during chronic viral infection and in tumors. Despite Hmgb2^{-/-} CD8⁺ T cells expressing TCF-1 and TOX, these master regulators were unable to sustain Tpex differentiation and long-term survival during persistent antigen. Furthermore, HMGB2 also had a cell-intrinsic function in the differentiation and function of memory CD8⁺ T cells after acute viral infection. Our findings show that HMGB2 is a key regulator of CD8⁺ T cells and is an important molecular target for T cell-based immunotherapies.

### Results

### HMGB2 Expression is Upregulated and Sustained in Virus-Specific CD8⁺ T Cells

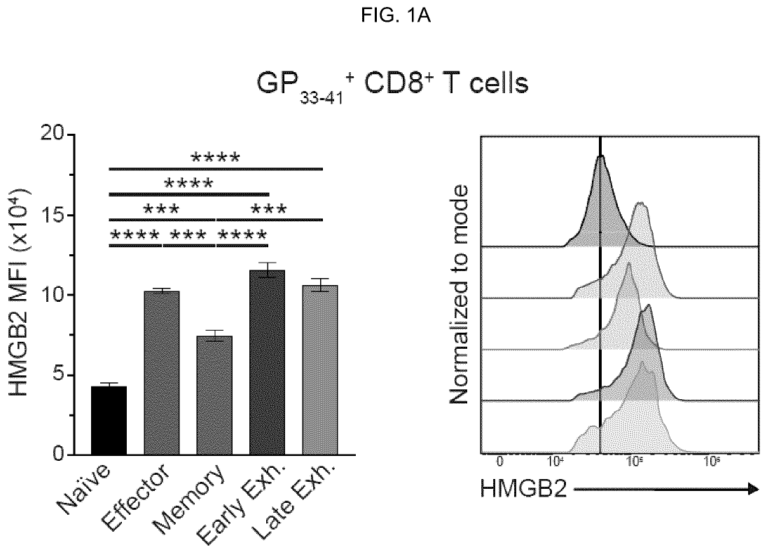

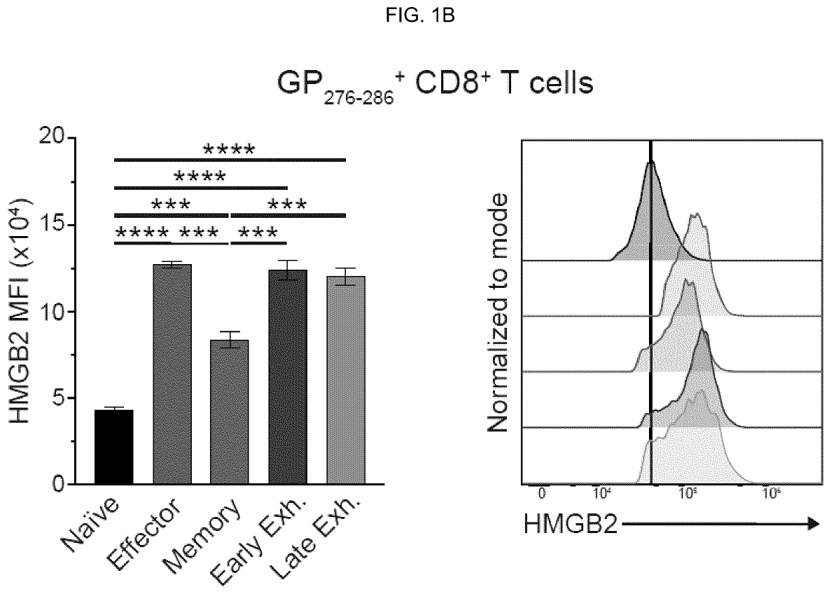

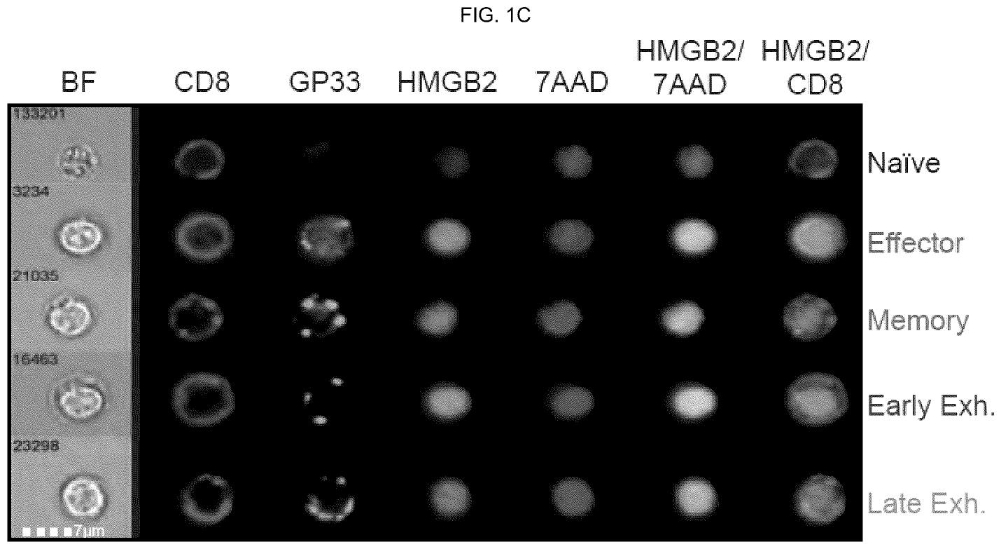

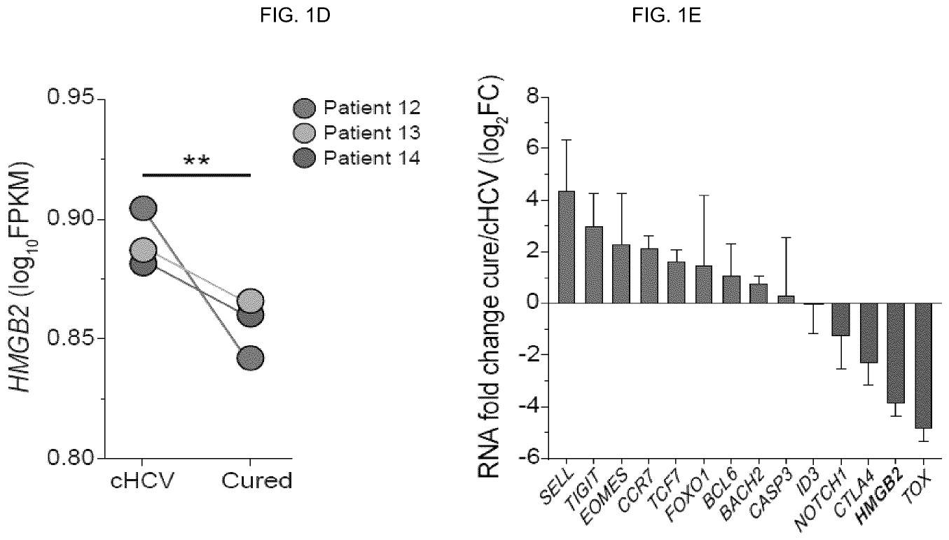

To assess how HMGB2 is regulated in virus-specific CD8⁺ T cells, we evaluated its expression in naïve, effector, memory, and early and late exhausted T cells. We infected wild-type (WT) mice with either LCMV Armstrong (Arm) or Clone 13 (C113) to induce an acute or chronic viral infection, respectively, and measured HMGB2 levels in MHC class I tetramer⁺ virus-specific CD8⁺ T cells. We detected HMGB2 expression in naïve CD8⁺ T cells that was upregulated in GP₃₃₋₄₁⁺ CD8⁺ effector and memory T cells (FIG. 1a). We observed upregulation of HMGB2 in early exhausted T cells (8 dpi C113) that was sustained in late exhausted T cells (30 dpi C113) (FIG. 1a). Furthermore, late exhausted GP₃₃₋₄₁⁺ T cells had significantly increased HMGB2 expression compared to naïve and memory T cells (FIG. 1a). We next evaluated HMGB2 levels in GP₂₇₆₋₂₈₆⁺ CD8⁺ T cells, and again observed increased levels in effector, memory, and exhausted T cells compared to naïve, with sustained HMGB2 expression in late exhausted CD8⁺ T cells (FIG. 1b). Lastly, we evaluated HMGB2 localization in GP₃₃₋₄₁⁺ CD8⁺ T cells using imaging flow cytometry. We observed nuclear localization of HMGB2, as shown by HMGB2 and nuclear 7-AAD colocalization staining (FIG. 1c). These findings showed nuclear localization of HMGB2 in virus-specific CD8⁺ T cells, with upregulated and sustained expression in memory and exhausted CD8⁺ T cells during viral infection.

HMGB2 is Upregulated in Hepatitis C-Specific CD8⁺ T Cells and Downregulated after Viral Clearance

Since exhausted T cells are also found during chronic viral infection in humans, we wanted to see how HMGB2 was regulated in patient exhausted T cells. Using previously published RNA-sequencing data²², we quantified HMGB2 levels in Hepatitis C (HCV) virus-specific CD8⁺ T cells isolated from patients during chronic infection and after viral clearance with direct-acting antiviral (DAA) therapy. Data collected from three separate patients showed high HMGB2 expression in T cells during chronic HCV infection, with significantly decreased levels after HCV clearance (FIG. 1d). We next analyzed expression of exhaustion associated genes in these patient HCV-specific CD8⁺ T cells before and after DAA-mediated cure (FIG. 1e). We found that expression of SELL, TIGIT, EOMES, CCR7, TCF7, FOXO1, BCL6, BACH2, and CASP3 were increased whereas NOTCH1, CTLA4, HMGB2, and TOX were decreased in T cells after viral clearance (FIG. 1e). These findings showed that HMGB2 was increased in chronic HCV patient exhausted CD8⁺ T cells and decreased after viral clearance, suggesting that HMGB2 may play a role in exhausted T cells in humans.

Hmgb2^{-/-} CD8⁺ T Cells Differentiate into Effector and Memory T Cells During Acute Viral Infection

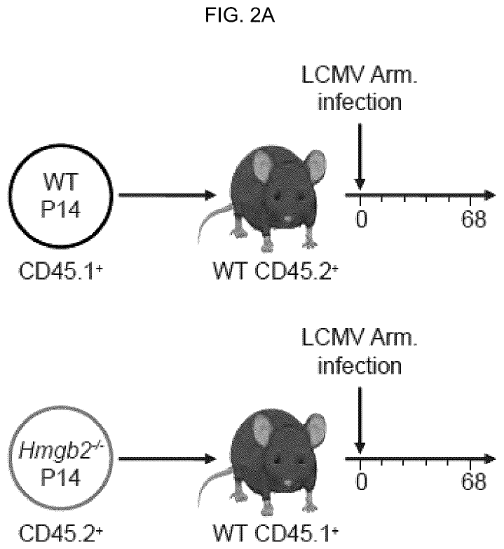

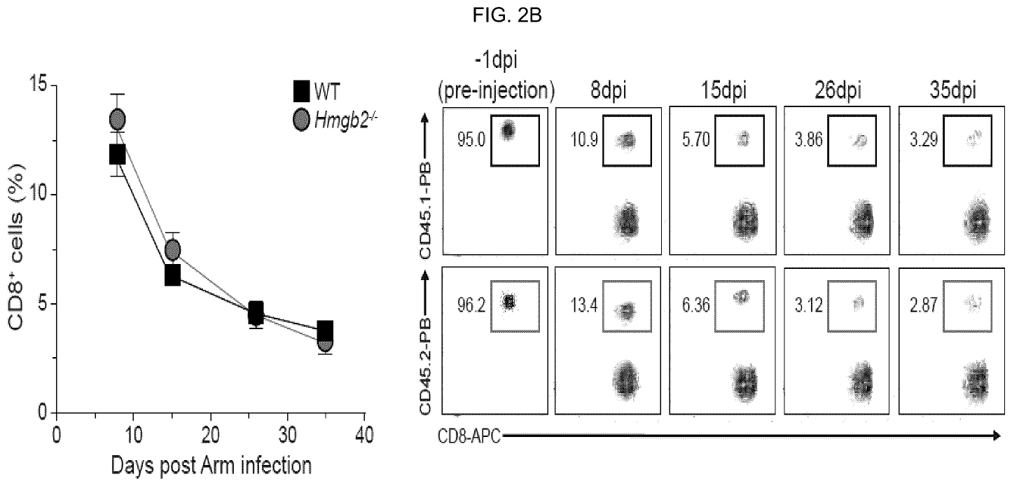

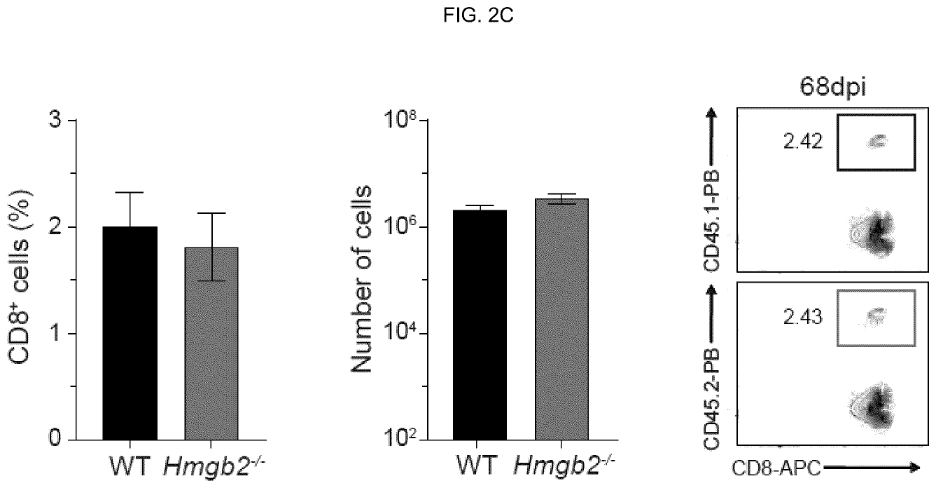

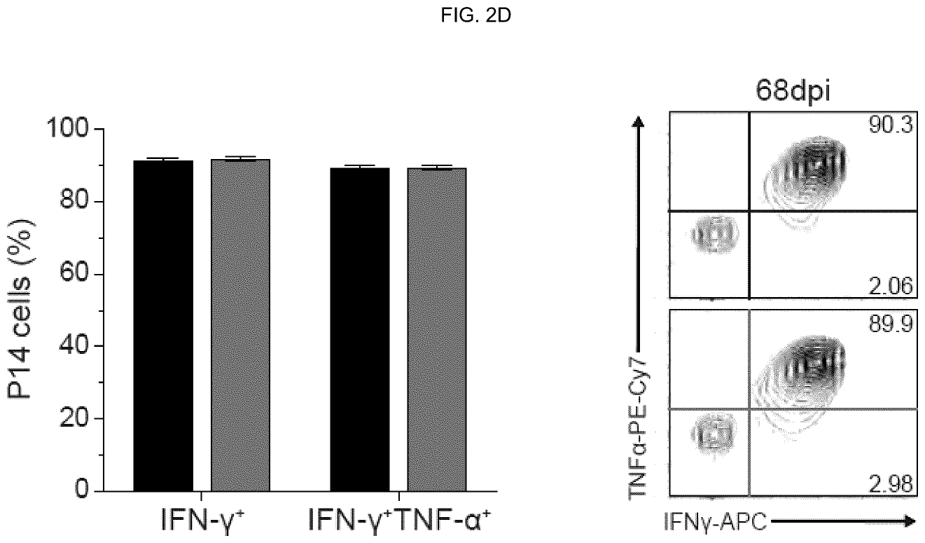

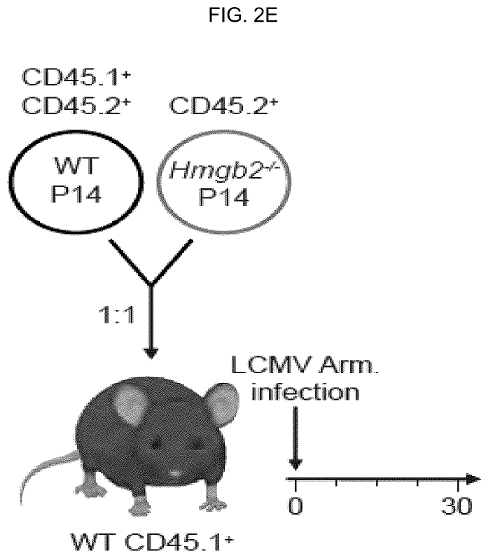

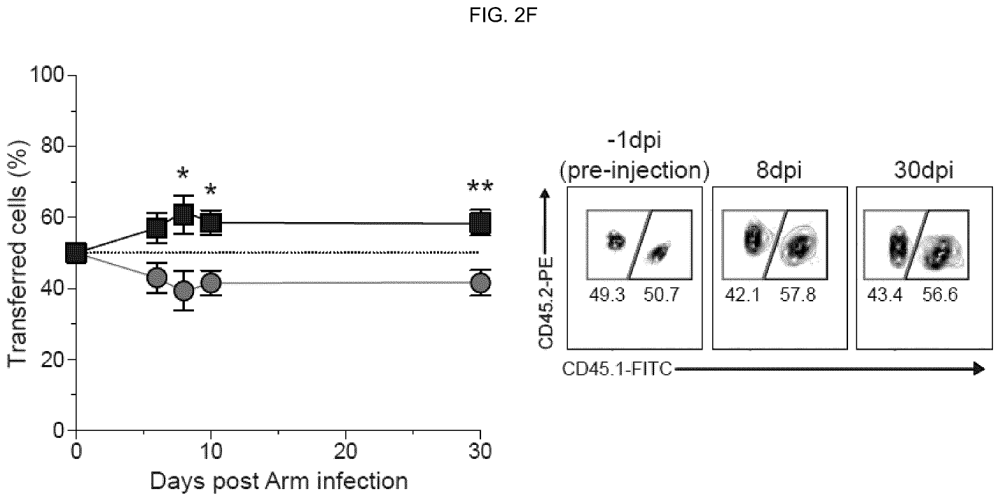

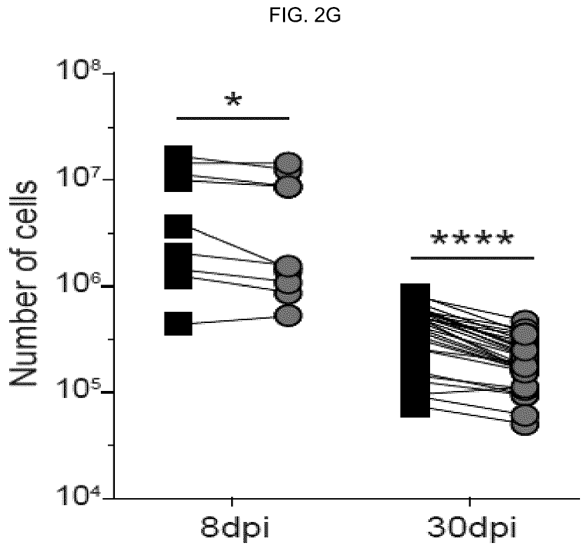

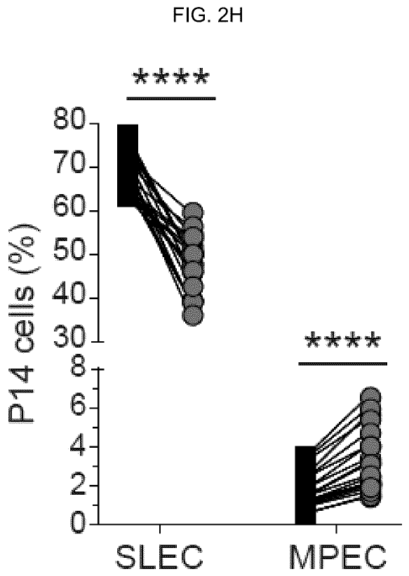

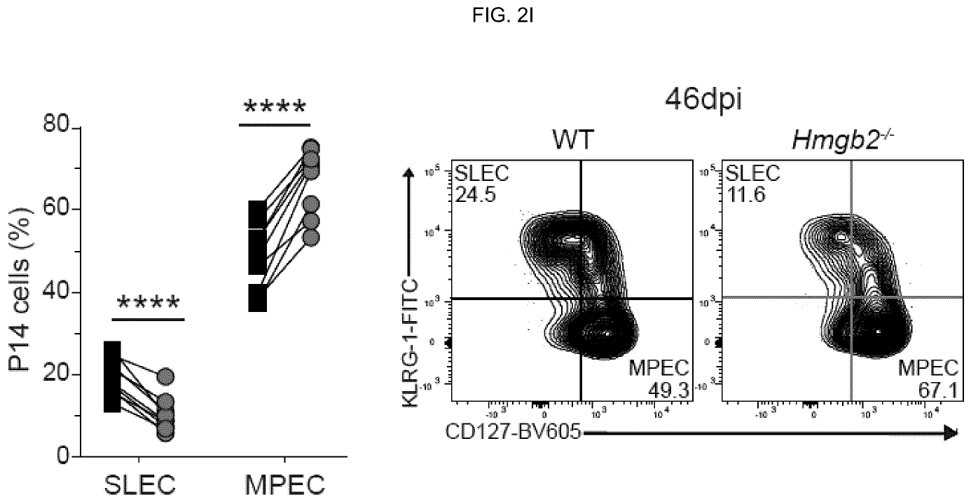

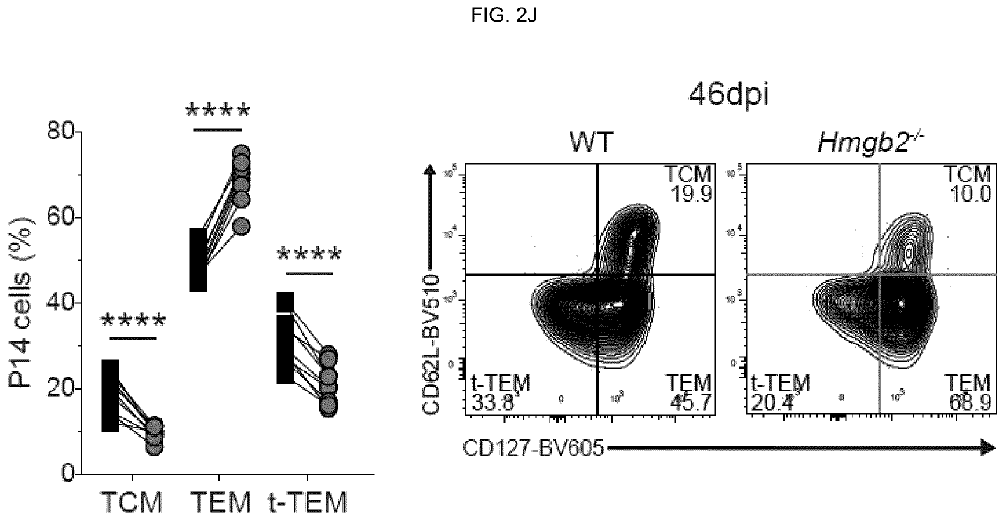

Considering the expression of HMGB2 was increased in effector and memory CD8⁺ T cells, we next determined the cell-intrinsic role of HMGB2 in virus-specific T cells during acute LCMV infection. Small numbers (1×10³ cells) of congenically marked WT or Hmgb2^{-/-} P14 CD8⁺ T cells, which are TCR transgenic T cells specific for the GP₃₃₋₄₁ peptide of LCMV, were adoptively transferred into separate congenically mismatched WT mice, which were then infected with LCMV Arm (FIG. 2a). A gating strategy for this approach is shown (FIG. S1a). We observed similar expansion, contraction, and memory CD8⁺ T cell formation of both WT and Hmgb2^{-/-} P14 T cells throughout acute infection (FIG. 2b). Furthermore, similar frequencies and numbers of WT and Hmgb2^{-/-} P14 T cells were found in spleens at 68 dpi (FIG. 2c).

We next evaluated functionality of WT and Hmgb2^{-/-} P14 T cells by ex vivo GP₃₃₋₄₁ peptide stimulation and observed similar frequencies of IFN-γ⁺ and IFN-γ⁺TNF-α⁺ cells at 68 dpi and GranzymeB⁺ cells at 8 dpi (FIG. 2d, S2a). To investigate T cell responses within the same host, we next co-transferred small numbers (1×10³ each) of congenically marked WT and Hmgb2^{-/-} P14 T cells at a 1:1 ratio into congenically mismatched WT mice, followed by Arm infection (FIG. 2e). Despite injection at a 1:1 ratio, we observed slightly decreased frequencies of Hmgb2^{-/-} P14 T cells compared to WT (FIG. 2f). We also observed significantly decreased numbers of splenic Hmgb2^{-/-} P14 T cells compared to WT at both 8 and 30 dpi (FIG. 2g). These findings showed Hmgb2^{-/-} CD8⁺ T cells expand after acute LCMV infection, and although present at slightly lower frequencies than WT, survive to seed the memory T cell pool.

HMGB2 Regulates Memory CD8⁺ T Cell Differentiation after Acute Infection

We next evaluated the differentiation of Hmgb2^{-/-} P14 T cells by examining KLRG-1 and IL-7Rα, expression to delineate KLRG1^{hi}IL-7Rα^{lo} short-lived effector (SLEC) and KLRG1^{lo}IL-7Rα^{hi} memory precursor effector cells (MPEC). At 8 dpi, we observed decreased SLECs in Hmgb2^{-/-} P14 T cells (FIG. 2h) and by 46 dpi, we found significantly decreased frequencies of SLECs and increased frequencies of MPECs in Hmgb2^{-/-} P14 T cells (FIG. 2i). Since we found more Hmgb2^{-/-} MPECs compared to WT but significantly less overall Hmgb2^{-/-} memory T cell numbers (FIG. 2g), we investigated apoptosis within this population. We found Hmgb2^{-/-} MPECs had significantly higher frequencies of total apoptotic cells by active Caspase3 and propidium iodide (PI) staining at 46 dpi (FIG. S2b).

To further characterize the differentiation of memory Hmgb2^{-/-} P14 T cells, we examined effector memory (Tem), central memory (Tcm) and terminal-Tem (t-Tem) T cell populations based on CD62L vs CD127 expression³⁵. Hmgb2^{-/-} P14 T cells had significantly lower frequencies of both Tcm and t-Tem cells, and increased frequencies of Tem cells compared to WT at 46 dpi (FIG. 2j). Finally, to confirm the loss of Hmgb2^{-/-} P14 T cells was not due to compromised development of naïve CD8⁺ T cells, we characterized naïve WT and Hmgb2^{-/-} P14 T cells from spleens and lymph nodes of uninfected mice. We found no differences in expression of naïve T cell markers (CD127, CD62L, CCR7) or in frequencies of naïve vs activated T cells in lymph nodes (FIG. S3a, b) and spleens (FIG. S3c, d). We also found no differences in numbers of CD8⁺ T cells between WT and Hmgb2^{-/-} mice (FIG. S3e). Together, these findings showed that HMGB2 regulated the survival and differentiation of memory CD8⁺ T cells during acute viral infection.

### Cell-Intrinsic HMGB2 Expression is Essential for Long-Term Maintenance of Exhausted CD8⁺ T Cells

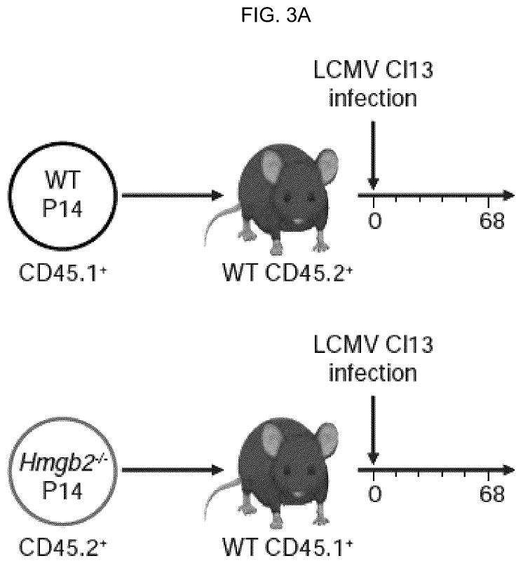

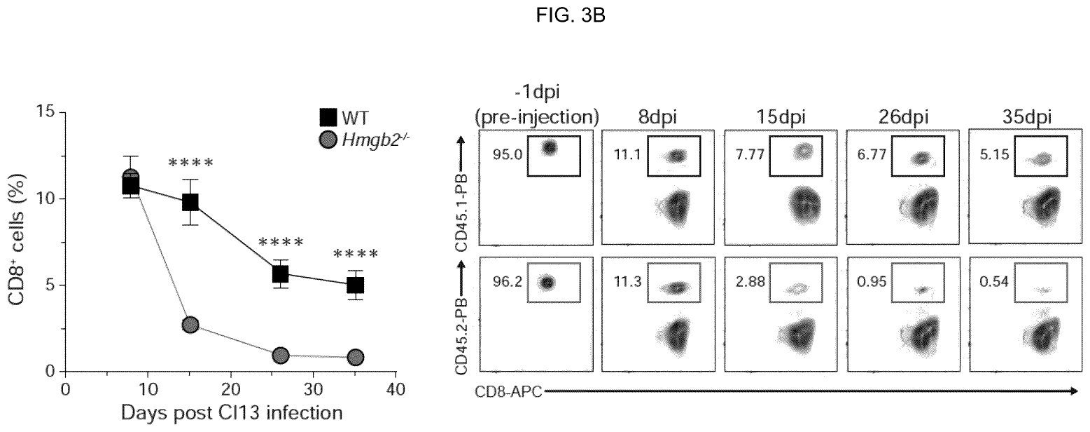

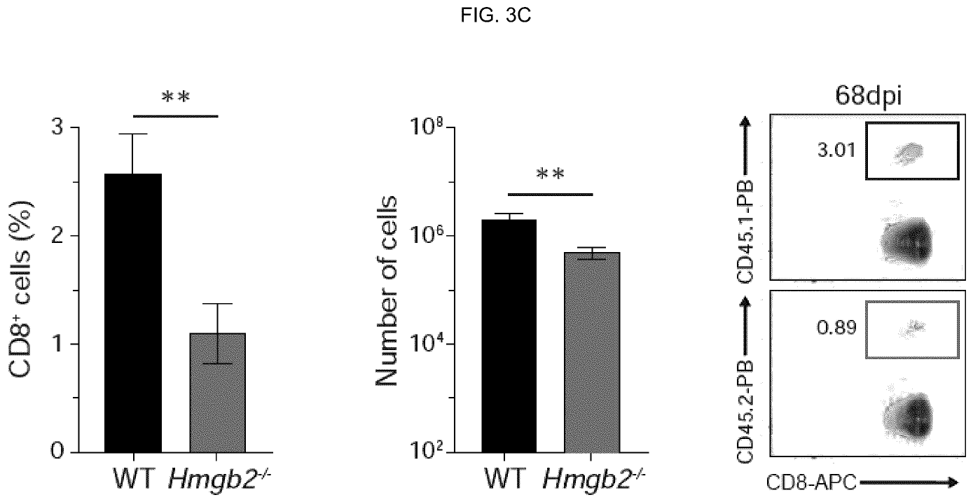

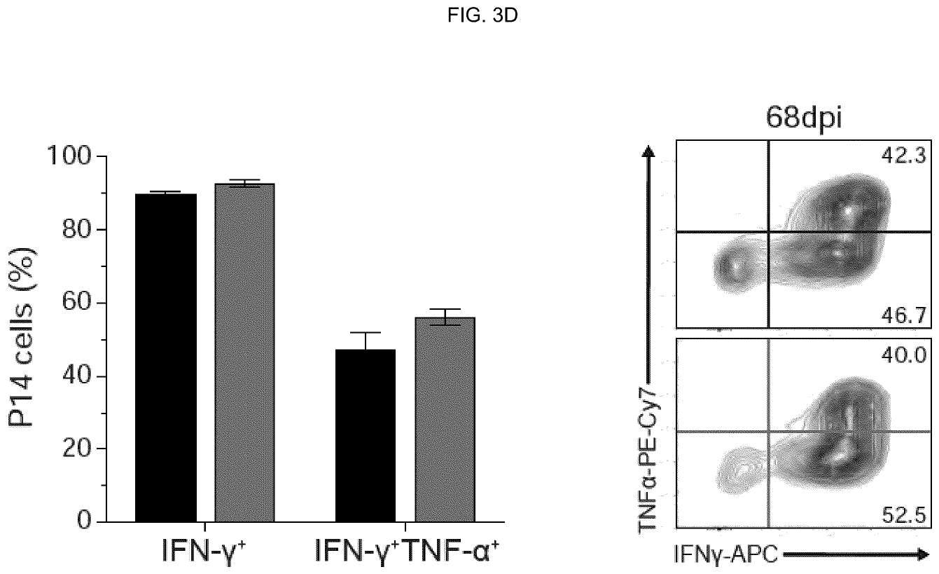

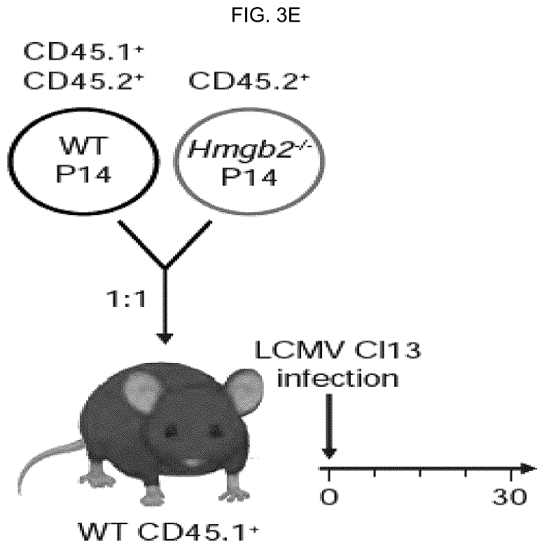

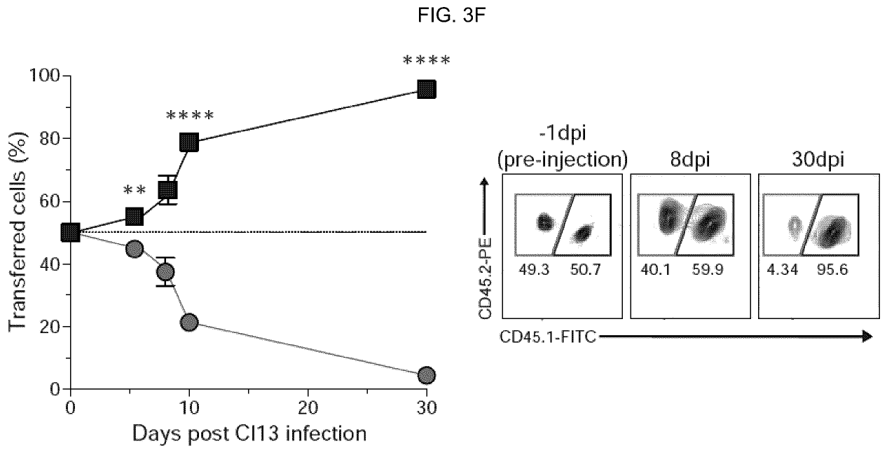

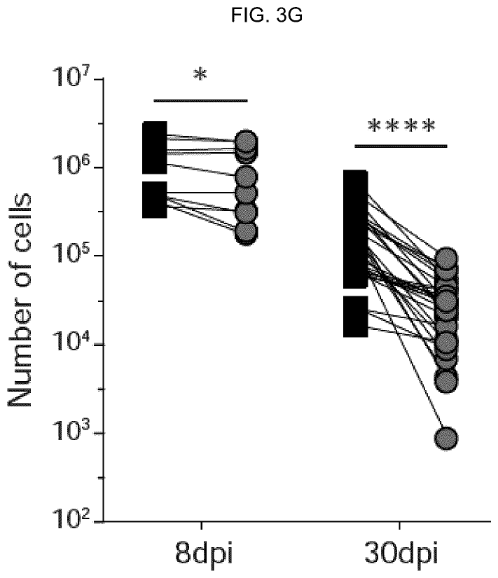

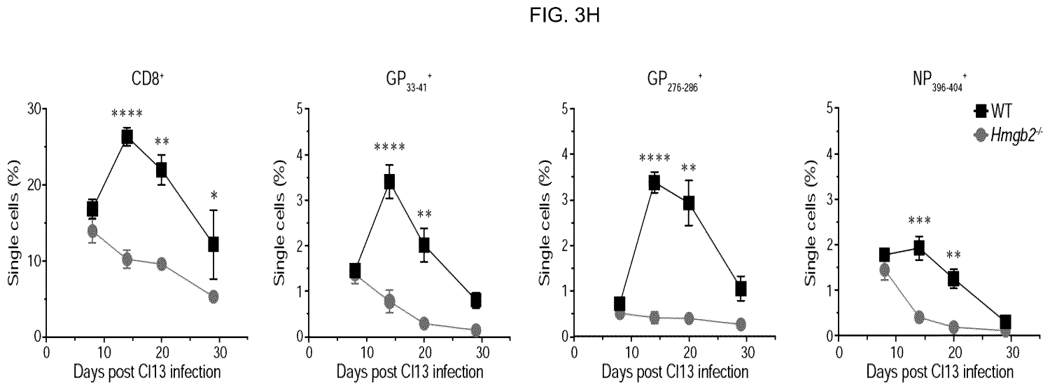

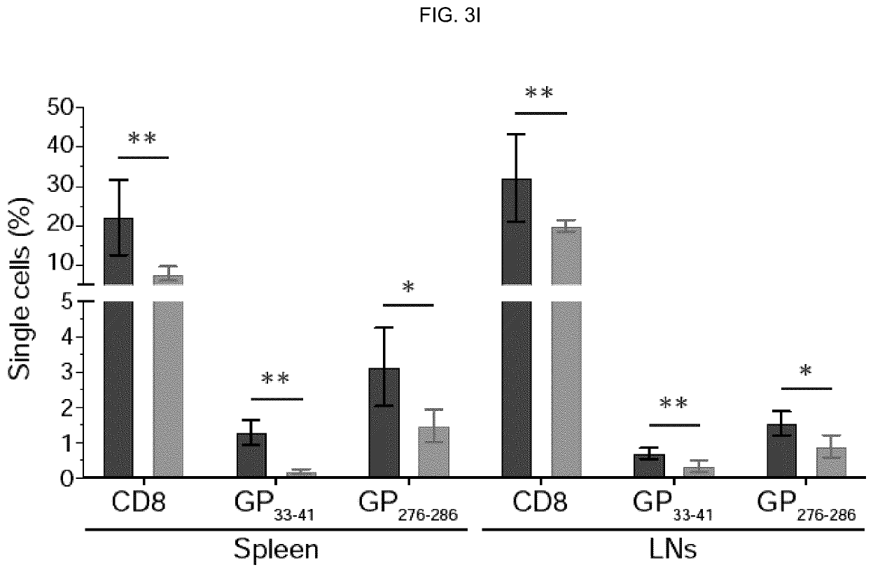

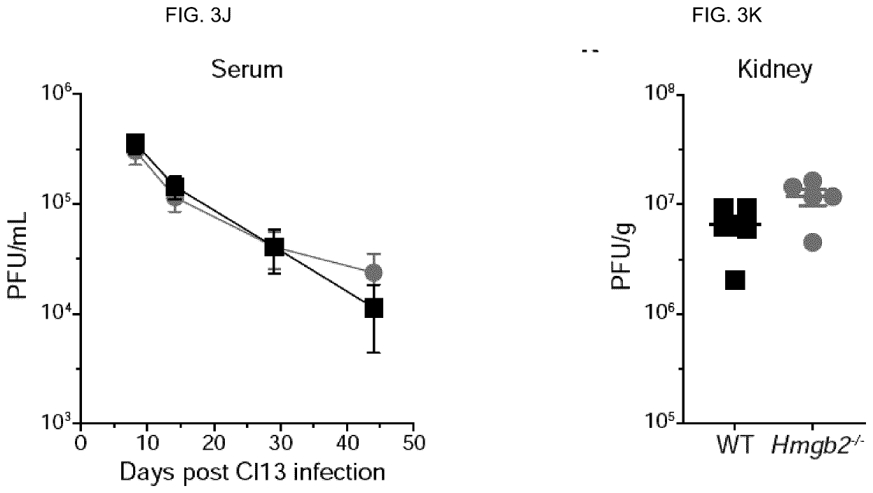

Since HMGB2 expression was increased and sustained in exhausted CD8⁺ T cells, we next determined the cell-intrinsic role of HMGB2 in virus-specific T cells during chronic LCMV infection. Small numbers (1×10³ cells) of congenically marked WT or Hmgb2^{-/-} P14 T cells were adoptively transferred into congenically mismatched WT mice and infected with LCMV C113 (FIG. 3a). We observed similar expansion of both WT and Hmgb2^{-/-} P14 T cells at 8 dpi, but Hmgb2^{-/-} P14 T cells had an accelerated decline and were present at significantly lower frequencies compared to WT by 15 dpi (FIG. 3b). Furthermore, we observed significantly decreased frequencies and numbers of Hmgb2^{-/-} P14 T cells in spleens at 68 dpi (FIG. 3c). We next evaluated the functionality of WT and Hmgb2^{-/-} P14 T cells and observed similar frequencies of IFN-γ⁺ and IFN-γ⁺TNF-α⁺ T cells at 68 dpi and GranzymeB⁺ cells at 8 dpi (FIG. 3d, S4a). To investigate cell-intrinsic HMGB2 function during chronic infection, we co-transferred WT and Hmgb2^{-/-} P14 T cells at a 1:1 ratio into WT mice and then infected with C113 (FIG. 3e). Despite starting at a 1:1 ratio, we observed a significant decrease in frequencies of Hmgb2^{-/-} P14 T cells compared to WT cells as early as 8 dpi (FIG. 3f). Additionally, we observed significantly decreased numbers of Hmgb2^{-/-} P14 T cells compared to WT at 8 and 30 dpi (FIG. 3g). We next evaluated co-inhibitory receptor expression and found that Hmgb2^{-/-} CD8⁺ T cells were increased in PD-1⁺TIM-3⁺ cells throughout C113 infection (FIG. S4b). Furthermore, we observed higher PD-1 expression in Hmgb2^{-/-} P14 T cells at 26 and 35 dpi (FIG. S4c). Since it has been established TOX is required for the generation of exhausted T cells during C113 infection^{36, 37, 38}, we next asked if the loss of exhausted Hmgb2^{-/-} P14 T cells was due to decreased TOX expression compared to WT. Significantly, we found Hmgb2^{-/-} P14 T cells had both similar and increased frequencies of TOX⁺ cells compared to WT, but had impaired generation of exhausted T cells (FIG. S4d).

Since we saw diminished maintenance of exhausted Hmgb2^{-/-} P14 T cells, we wanted to investigate the impact of HMGB2 deletion on other virus-specific T cell clones by infecting WT and Hmgb2^{-/-} mice with C113. By 8 dpi there was similar expansion of CD8⁺, GP₃₃₋₄₁⁺, GP₂₇₆₋₂₈₆⁺, and NP₃₉₆₋₄₀₄⁺ T cells in both WT and Hmgb2^{-/-} mice, but by 14 dpi and onwards there were significantly less virus-specific CD8⁺ T cells in Hmgb2^{-/-} mice (FIG. 3h). At 44 dpi, there were significantly decreased frequencies of CD8⁺, GP₃₃₋₄₁⁺, and GP₂₇₆₋₂₈₆⁺ T cells in spleens and lymph nodes of Hmgb2^{-/-} mice compared to WT (FIG. 3i). Throughout the course of infection there were no differences in viral titers in the serum, but we observed a trend towards increased titers in the kidneys of Hmgb2^{-/-} mice at 46 dpi (FIG. 3j, k). Together, these findings showed HMGB2 was essential for the formation and long-term maintenance of exhausted CD8⁺ T cells during chronic viral infection.

### HMGB2 Regulates the Transcriptional Signature of Progenitor Exhausted CD8⁺ T Cells

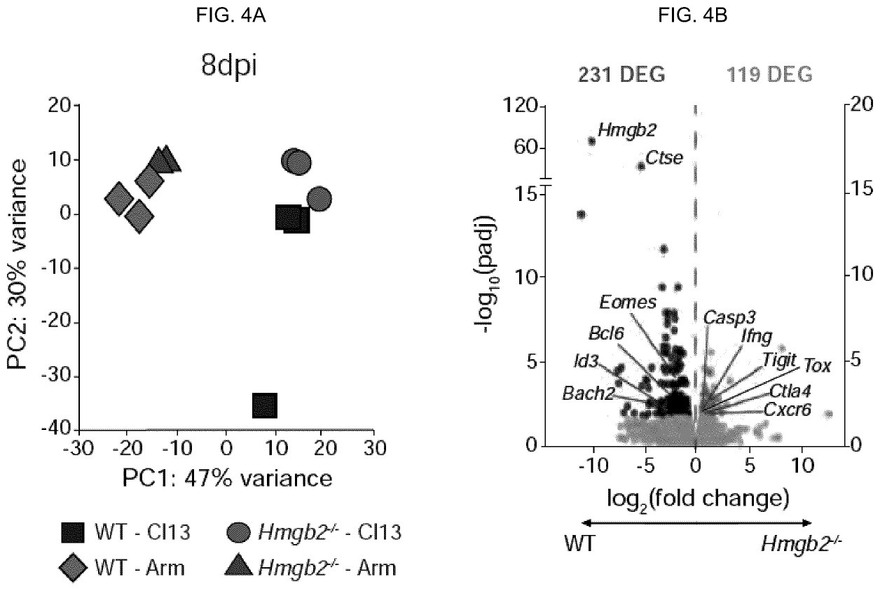

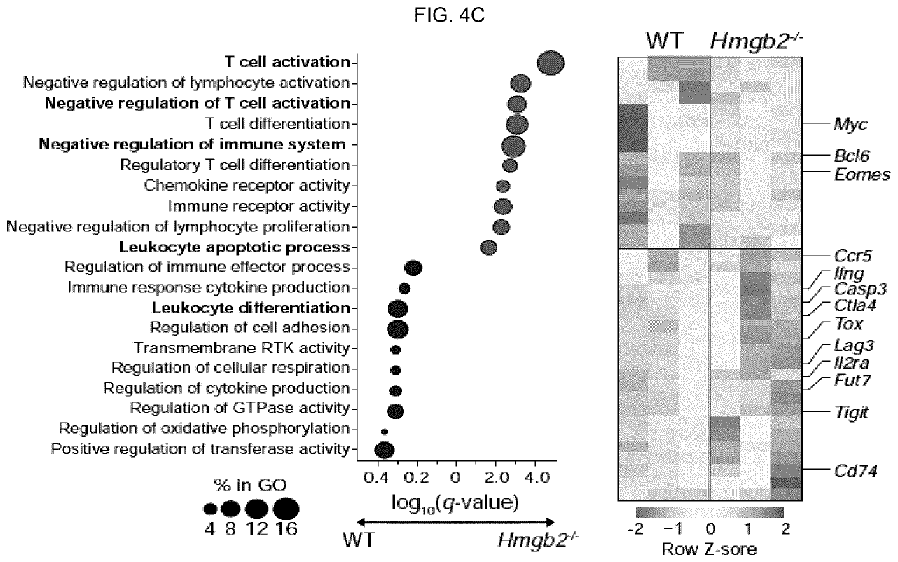

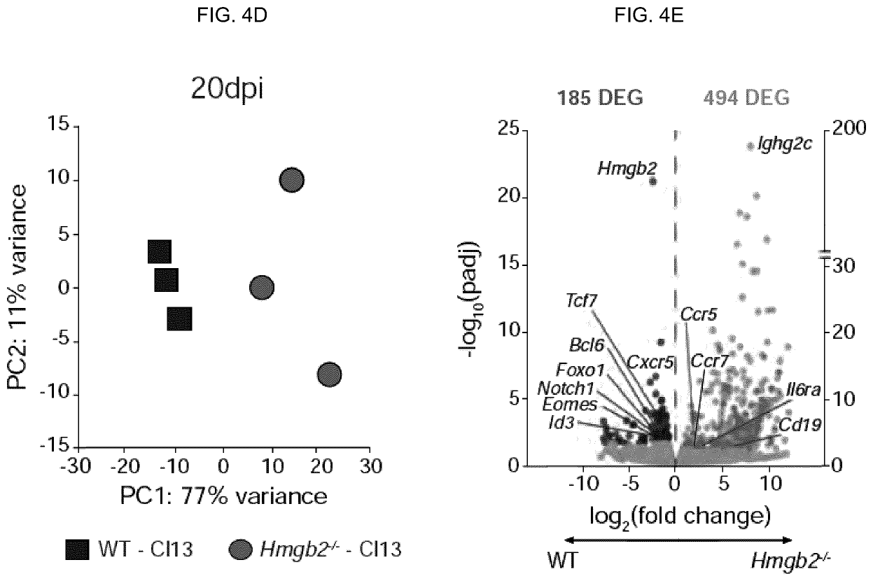

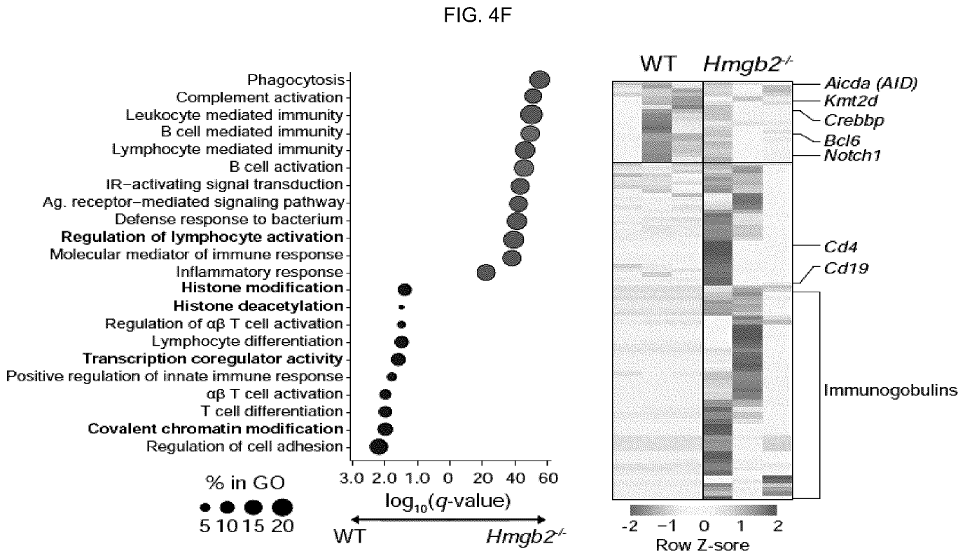

Since we found roles for HMGB2 in memory and exhausted CD8⁺ T cells, and HMGB2 is a chromatin modifier, we next investigated whether HMGB2 regulated the transcriptional landscape of virus-specific T cells. We performed RNA-sequencing of sorted WT and Hmgb2^{-/-} P14 T cells from Arm and C113 infected mice at 8 dpi. Principal component analysis (PCA) showed the type of infection accounted for transcriptional differences across PC1 (47% variance), while Hmgb2 expression accounted for transcriptional changes across PC2 (30% variance) (FIG. 4a). Overall, we observed fewer differentially expressed genes (DEG) between WT and Hmgb2^{-/-} P14 T cells during Arm infection than C113. During Arm infection, analysis of DEG between WT and Hmgb2^{-/-} P14 T cells showed a total of 125, with 38 upregulated and 87 downregulated genes in Hmgb2^{-/-} P14 T cells (FIG. S5a, b). Conversely, there were 350 DEG with 119 upregulated and 231 downregulated genes in Hmgb2^{-/-} P14 T cells during C113 infection (FIG. 4b). In WT P14 T cells, we observed increased expression of genes promoting stem-like progenitor exhausted T cell (Tpex) differentiation (Eomes, Bcl6, Id3, Bach2), whereas Hmgb2^{-/-} P14 T cells had increased expression of genes associated with terminal exhausted T cells (Tex) (Casp3, Tigit, Tox, Ctla4, Ifng) (FIG. 4b)³⁹. To identify the biological significance of these DEG during C113 infection, we performed gene ontology (GO) analysis. We found WT P14 T cells were enriched for pathways associated with (i) leukocyte differentiation; (ii) regulation of cellular respiration and oxidative phosphorylation; and (iii) transferase activity (FIG. 4c). In contrast, Hmgb2^{-/-} P14 T cells were enriched for pathways associated with (i) negative regulation of T cell activation; (ii) immune receptor activity; and (iii) apoptosis (FIG. 4c). These findings showed Hmgb2^{-/-} CD8⁺ T cells are enriched for gene signatures and pathways of terminal exhaustion during chronic viral infection.

### HMGB2 Required for Expression of Tpex Genes, Including Genes Regulated by TCF-1

To further investigate the transcriptional changes driven by HMGB2 in CD8⁺ T cells during chronic infection, we performed RNA-sequencing on WT and Hmgb2^{-/-} P14 T cells sorted from C113 infected mice at 20 dpi. PCA showed clear separation of WT and Hmgb2^{-/-} P14 T cells, with Hmgb2 expression accounting for the transcriptional differences across PC1 (77% variance) (FIG. 4d). There were 679 DEG, with 494 upregulated and 185 downregulated in Hmgb2^{-/-} P14 T cells (FIG. 4e). Similar to 8 dpi C113, we observed increased expression of Tpex associated genes in WT P14 T cells (Tcf7, Cxcr5, Bcl6, Foxo1, Eomes, Id3) compared to Hmgb2^{-/-} P14 T cells (FIG. 4e)³⁹. Hmgb2^{-/-} P14 T cells expressed a dysregulated gene expression program, including non-CD8⁺ T cell lineage associated genes (Ighg2c, Cd19) (FIG. 4e). GO term analysis showed WT P14 T cells were enriched for pathways associated with (i) histone modification; (ii) histone deacetylation; (iii) lymphocyte differentiation; (iv) transcription coregulator activity; and (v) covalent chromatin modification (FIG. 4f). In contrast, Hmgb2^{-/-} P14 T cells were enriched for pathways associated with (i) phagocytosis; (ii) complement activation; (iii) B cell activation; (iv) regulation of lymphocyte activation; and (v) inflammatory responses (FIG. 4f).

Since HMGB proteins are also known to modulate transcription factor binding, we next used Ingenuity Pathway Analysis (IPA)⁴⁰ to investigate any transcriptional regulators modified by HMGB2 that may be responsible for the DEG between WT and Hmgb2^{-/-} P14 T cells at 20 dpi C113. Upstream causal network analysis identified TCF-1 as a possible master regulator of the 20 dpi C113 DEG, with the TCF-1 regulator network predicated to be significantly inhibited in exhausted Hmgb2^{-/-} P14 T cells (activation z-score=−3.236, network bias-corrected p-value=0.00001) (FIG. S6a). Of the 679 DEG regulated by HMGB2 at 20 dpi C113, 117 are downstream targets of TCF-1, including Bcl6, Eomes, Id3, Foxo1, Notch1, and Tcf7 (FIG. S6a). This suggests that HMGB2 in CD8⁺ T cells regulates the Tpex transcription program during C113 infection, and may do so through modifying the TCF-1 transcriptional network.

Hmgb2^{-/-} CD8⁺ T Cells have Decreased Survival During Acute and Chronic Viral Infection

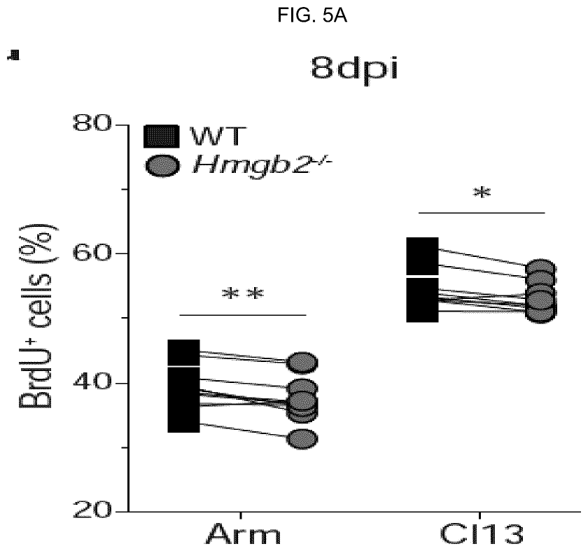

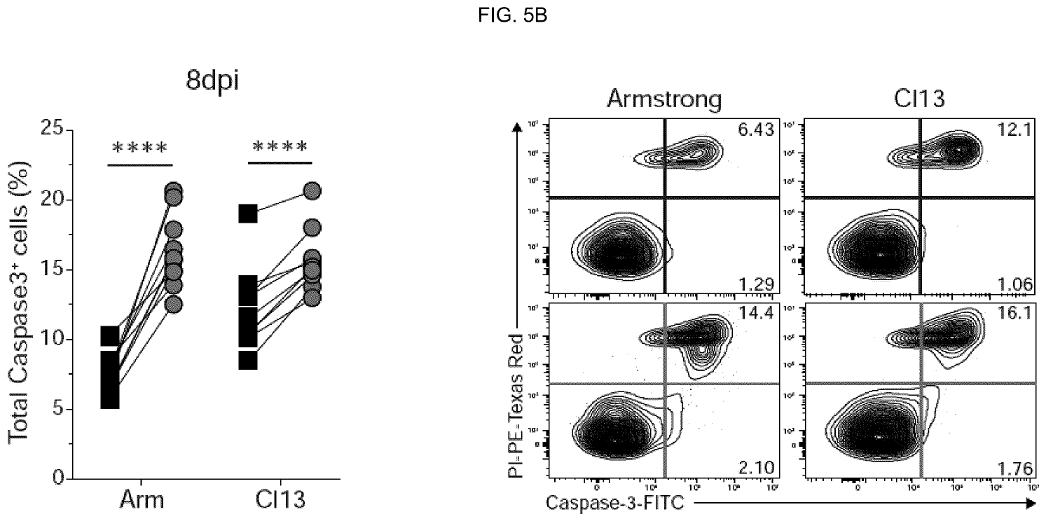

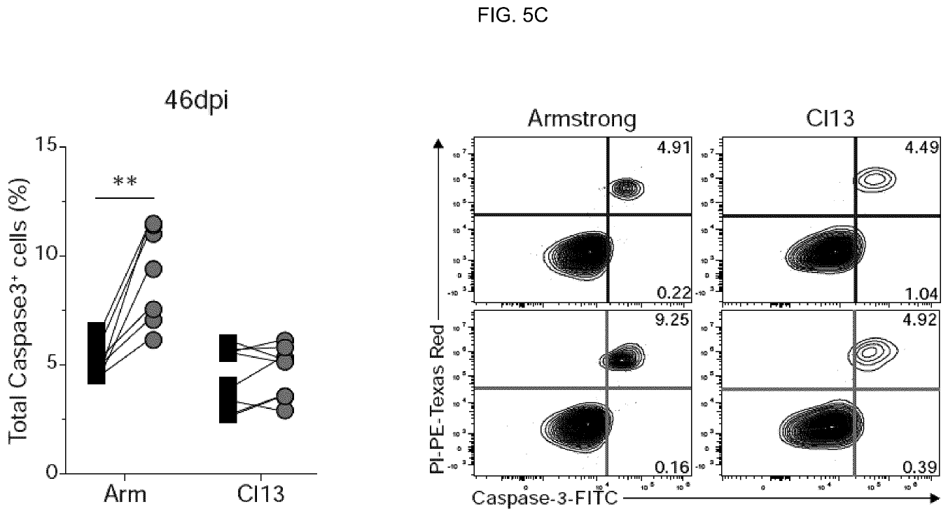

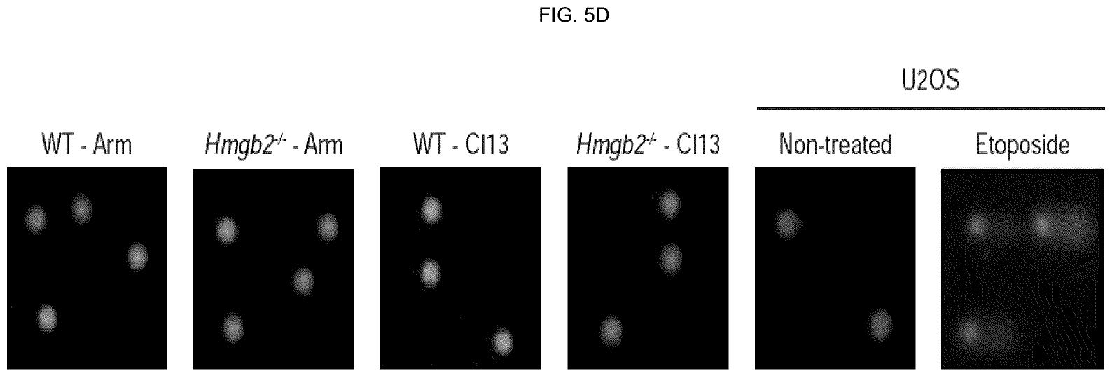

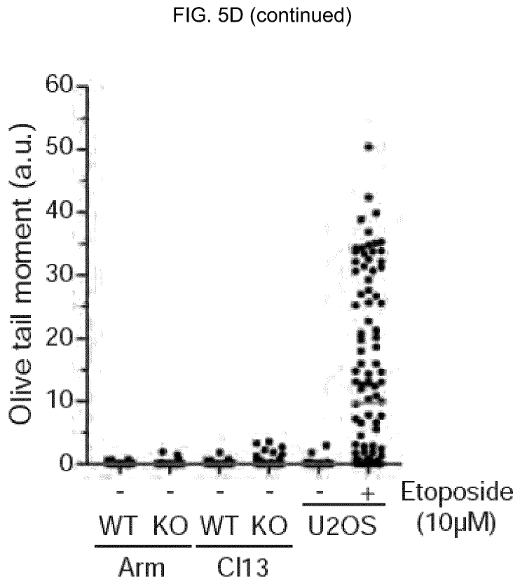

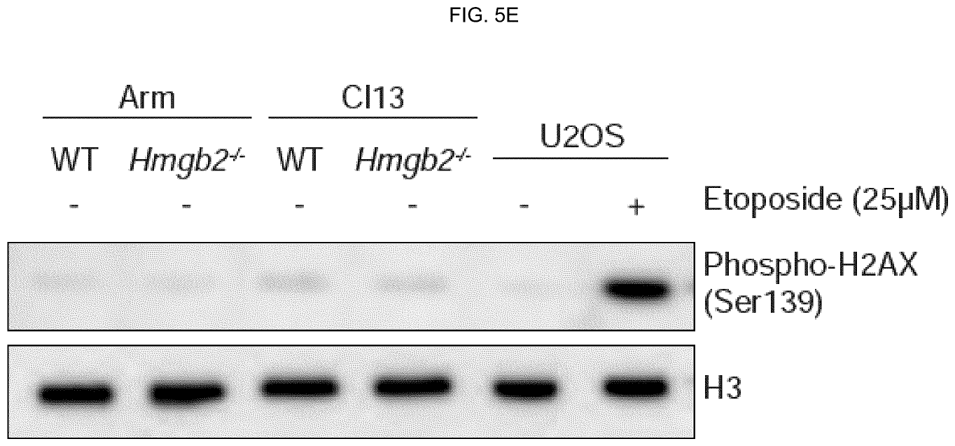

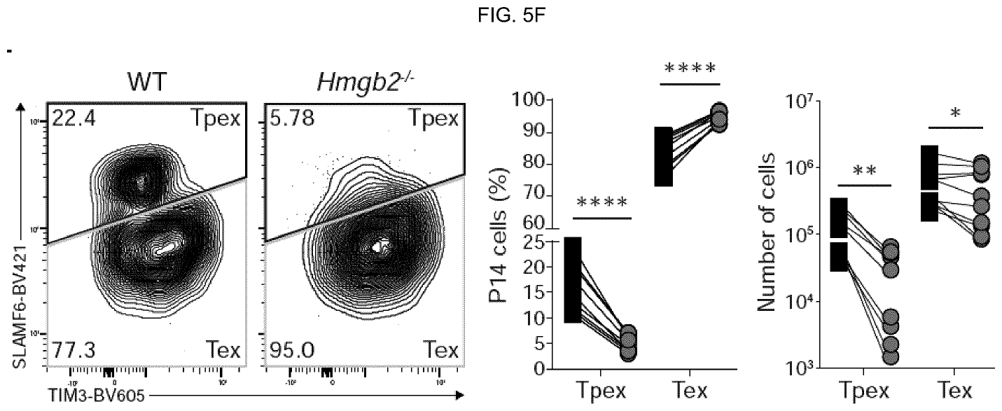

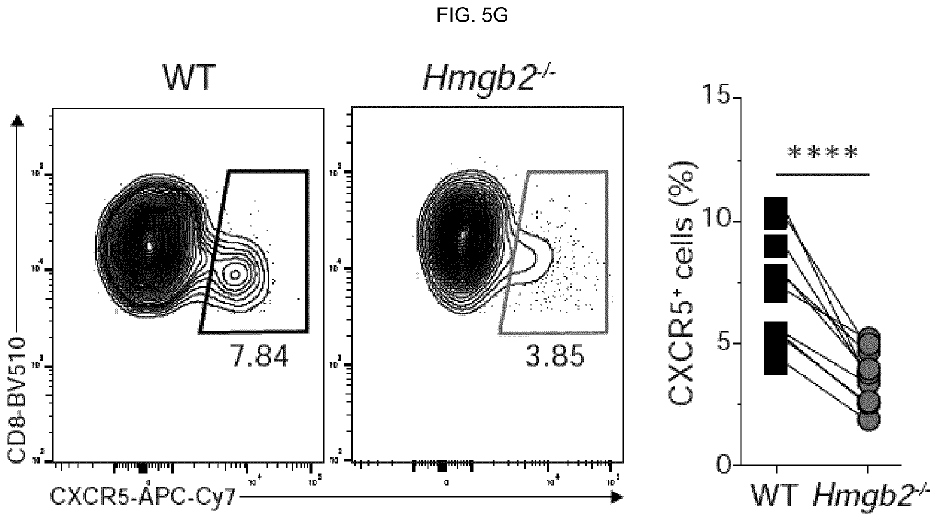

Since we found Hmgb2^{-/-} CD8⁺ T cells were significantly decreased during acute and chronic viral infection, we next assessed whether there were differences in their proliferation and/or survival. We co-transferred WT and Hmgb2^{-/-} P14 T cells at a 1:1 ratio into WT mice, followed by Arm or C113 infection. We first evaluated proliferation by in vivo BrdU incorporation and observed slightly decreased frequencies of BrdU⁺ Hmgb2^{-/-} P14 T cells compared to WT at both 8 dpi Arm and C113 (FIG. 5a). As expected, WT and Hmgb2^{-/-} P14 T cells from chronically infected mice had more proliferation than those from acute infection (FIG. 5a). Next, we evaluated survival by active Caspase3 and PI staining and observed increased frequencies of total Caspase3⁺ apoptotic Hmgb2^{-/-} P14 T cells compared to WT at 8 dpi Arm and C113 (FIG. 5b). We also found increased frequencies of total Caspase3⁺ apoptotic Hmgb2^{-/-} P14 T cells at 46 dpi Arm but saw minor differences at 46 dpi C113 (FIG. 5c). We next asked whether the increased apoptosis of Hmgb2^{-/-} P14 T cells was due to differences in DNA repair compared to WT cells. Using a comet assay to measure DNA damage⁴¹, we found no significant differences between WT and Hmgb2^{-/-} P14 T cells during both Arm and C113 infection (FIG. 5d). We also looked at phosphorylated-H2AX (Ser139), a marker of DNA damage⁴², and again found no differences between WT and Hmgb2^{-/-} P14 T cells during both Arm and C113 infection (FIG. 5e). These findings showed Hmgb2^{-/-} CD8⁺ T cells had decreased proliferation and survival compared to WT during both acute and chronic viral infection and did not show signs of DNA damage by 8 dpi.

### HMGB2 is Essential for the Differentiation of Progenitor Exhausted T Cells During Chronic Viral Infection

Recent findings have shown heterogeneity within the exhausted CD8⁺ T cell population, including the identification of Tpex and Tex cells. Since our sequencing data showed decreased expression of Tpex signature genes in Hmgb2^{-/-} P14 T cells compared to WT (Tcf7, Eomes, Bcl6, Id3), we wanted to determine changes in Tpex differentiation between WT and Hmgb2^{-/-} P14 T cells. At 8 dpi C113, we stained adoptively co-transferred P14 T cells with SLAMF6 and TIM-3 to identify Tpex (SLAMF6^{hi}TIM-3^{lo}) and Tex (SLAMF6^{lo} TIM-3^{hi}) cells. We found that Hmgb2^{-/-} P14 T cells had significantly diminished Tpex cell frequencies compared to WT (FIG. 5f). Consistent with the loss of the Tpex subset, Hmgb2^{-/-} P14 T cells were enriched for Tex-phenotype cells (FIG. 5f). Furthermore, we observed a significant decrease in the number of Hmgb2^{-/-} Tpex cells at both 8 and 46 dpi, while the numbers of Tex cells were similar (FIG. 5f, S7a). We also found significantly decreased CXCR5⁺ Hmgb2^{-/-} P14 T cells compared to WT, which is an additional surface marker of the Tpex population (FIG. 5g). Since we found decreased Tcf7 expression in Hmgb2^{-/-} P14 T cells and TCF-1 (Tcf7) is a key transcription factor driving Tpex cell differentiation, we investigated if the loss of Hmgb2^{-/-} Tpex cells was due to decreased TCF-1 expression compared to WT. Surprisingly, WT and Hmgb2^{-/-} P14 T cells had similar frequencies of TCF-1⁺ cells during C113 infection (FIG. S7b). In Hmgb2^{-/-} mice, we also found similar and even higher frequencies of TCF-1⁺ cells in Hmgb2^{-/-} CD8⁺, GP₃₃₋₄₁⁺, GP₂₇₆₋₂₈₆⁺, and NP₃₉₆₋₄₀₄⁺ T cells compared to WT mice during C113 infection (FIG. S7c).

Given the substantial loss of Hmgb2^{-/-} Tpex cells and the overall increased cell death of exhausted Hmgb2^{-/-} P14 T cells, we next investigated if Hmgb2^{-/-} Tpex cells were preferentially dying. At 8 dpi C113, we found no differences in Tpex cell death between WT and Hmgb2^{-/-} P14 T cells (FIG. S7d) but found a significant increase in apoptosis of Hmgb2^{-/-} Tpex cells compared to WT at 46 dpi (FIG. S7e).

Lastly, since we found HMGB2 played a critical role in the formation of Tpex cells, we wanted to evaluate the regulation of HMGB2 in this exhausted subset. Within WT P14 T cells, we found the highest expression of HMGB2 in the Tpex subset compared to the Tex (FIG. S7f). We also found the highest frequencies of Tpex cells within the WT P14 HMGB2⁺ population compared to the WT P14 HMGB2-population (FIG. S7g). Together, these findings showed that HMGB2 is a critical regulator of the differentiation and preservation of Tpex cells, which self-renew and maintain the exhausted T cell pool during C113 infection.

Hmgb2^{-/-} Memory CD8⁺ T Cells are Defective in their Recall to Secondary Infection

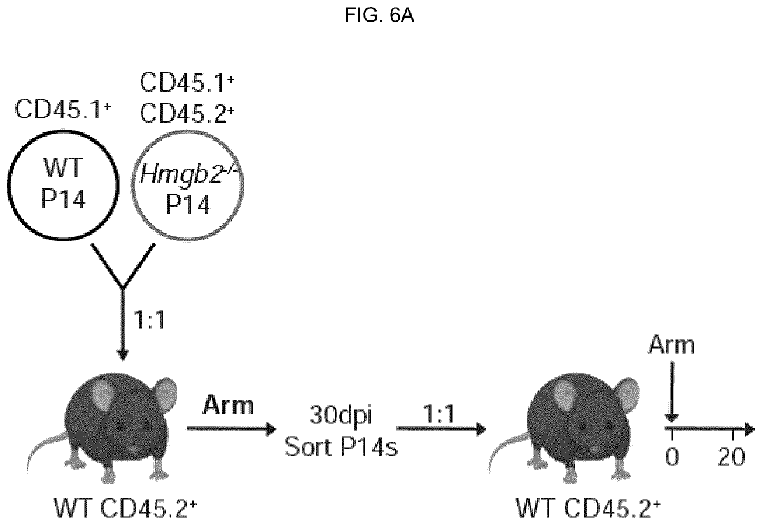

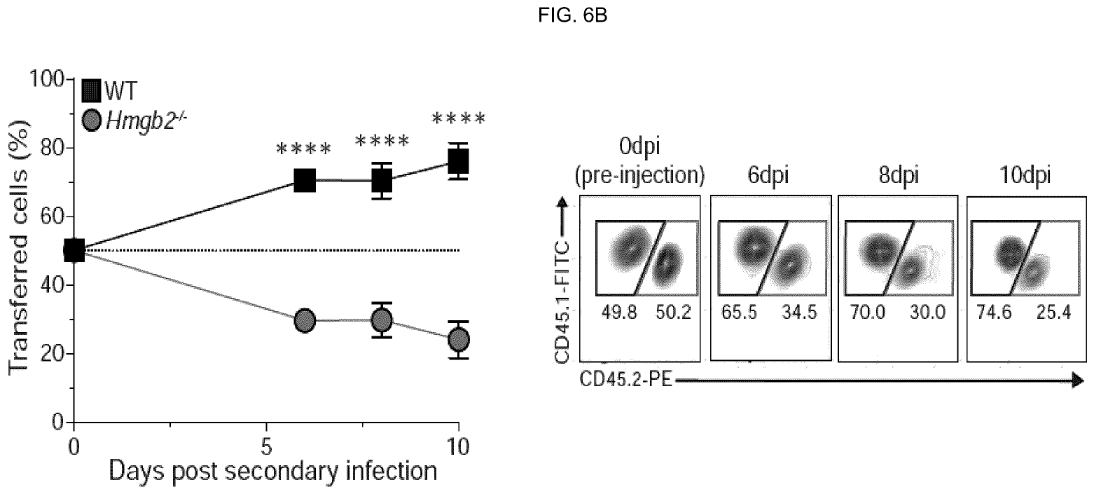

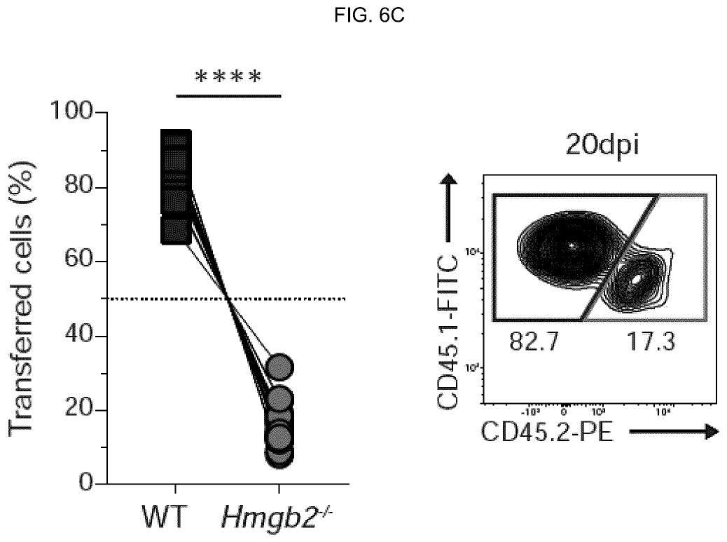

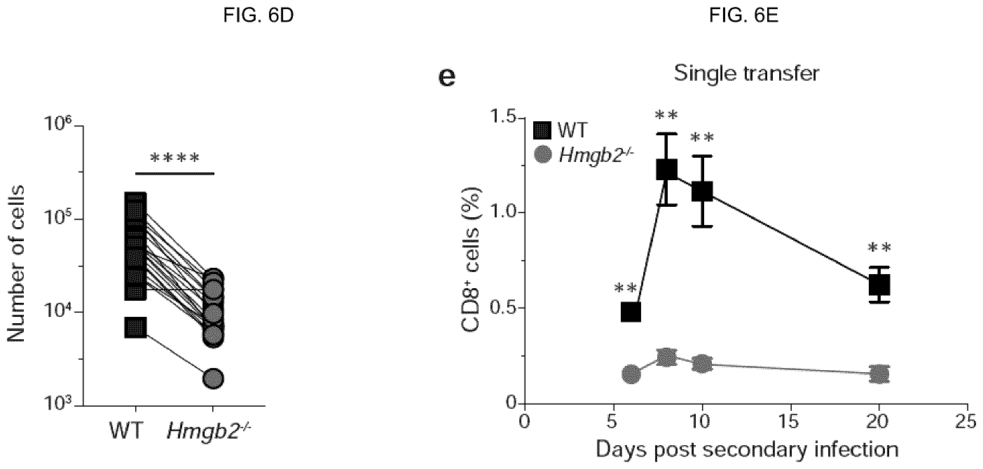

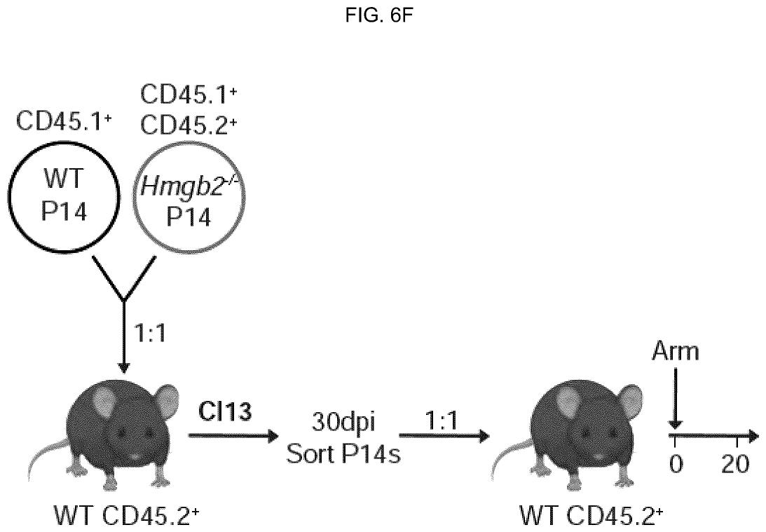

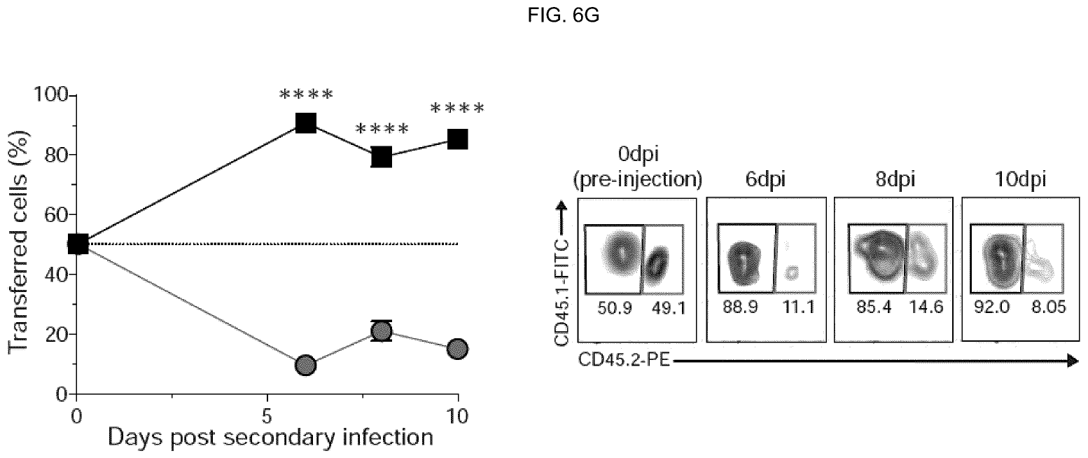

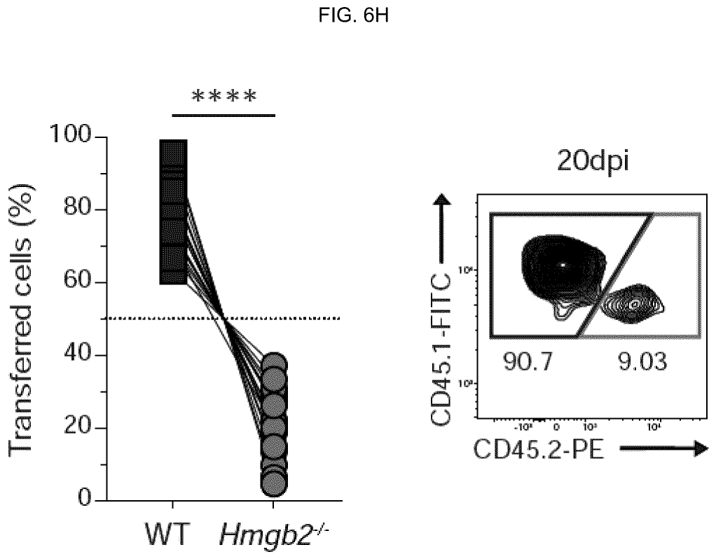

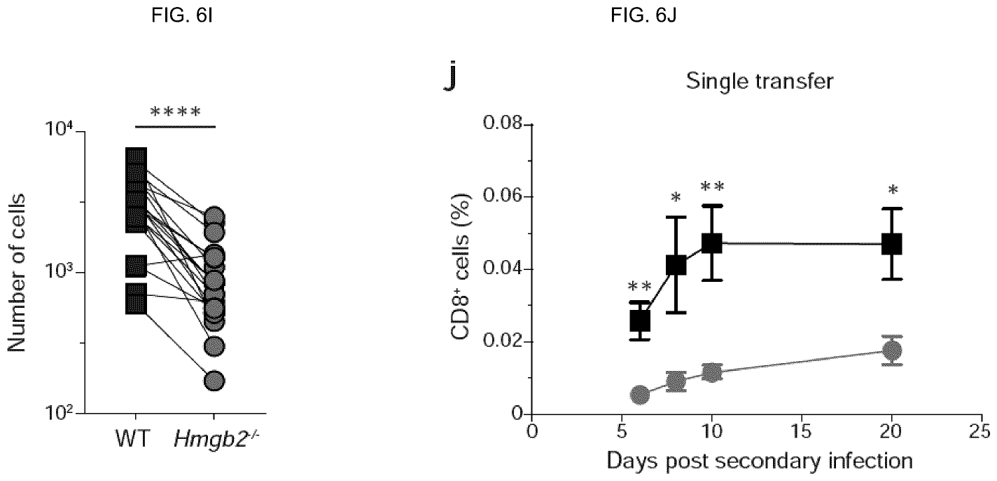

We found that Hmgb2^{-/-} P14 T cells survived to form memory T cells, but were deficient in Tcm formation (FIG. 2j). Since Tcm cells can self-renew to maintain the memory T cell pool and mediate memory T cell recall responses^{35, 43}, we next assessed whether HMGB2 played a role in the functionality of memory CD8⁺ T cells. Small numbers (1×10³ cells/each) of WT and Hmgb2^{-/-} P14 T cells were adoptively co-transferred at a 1:1 ratio into WT mice and subsequently infected with LCMV Arm (FIG. 6a). At 30 dpi, memory P14 T cells were sorted from spleens and co-transferred (2×10³ cells/each) at a 1:1 ratio into naïve WT mice, which were then infected with LCMV Arm (FIG. 6a). WT P14 T cells robustly re-expanded with secondary challenge, while Hmgb2^{-/-} P14 T cell frequencies were significantly decreased in the blood (FIG. 6b). Furthermore, we observed decreased frequencies and numbers of Hmgb2^{-/-} P14 T cells in spleens at 20 dpi (FIG. 6c, d). Similar results were seen when memory WT and Hmgb2^{-/-} P14 T cells were transferred separately into WT hosts and infected with LCMV Arm (FIG. 6e). Since TCF-1 is required to generate CD8⁺ memory T cell recall responses⁴³, we next investigated the expression of TCF-1 in Hmgb2^{-/-} P14 memory T cells prior to secondary infection. We found similar TCF-1⁺ frequencies between WT and Hmgb2^{-/-} P14 T cells both during and after acute Arm infection (FIG. S8a). These findings showed that HMGB2 expression was required for the re-expansion of memory CD8⁺ T cells during secondary viral infection.

Hmgb2^{-/-} Exhausted CD8⁺ T Cells are Decreased after Secondary Acute LCMV Challenge

We observed significantly decreased frequencies of Hmgb2^{-/-} Tpex cells during C113 infection, and since these cells drive the limited reinvigoration of exhausted T cells after secondary infections^{11, 12}, we next examined whether exhausted Hmgb2^{-/-} CD8⁺ T cells could respond to Arm infection. Small numbers (1×10³ cells/each) of WT and Hmgb2^{-/-} P14 T cells were adoptively co-transferred at a 1:1 ratio into WT mice and infected with C113 (FIG. 6f). At 30 dpi, exhausted P14 T cells were sorted from spleens and co-transferred (2×10′ cells/each) at a 1:1 ratio into new WT mice, which were then infected with LCMV Arm (FIG. 6f). Exhausted WT P14 T cells re-expanded with secondary challenge, while Hmgb2^{-/-} P14 T cells failed to expand and were at significantly decreased frequencies in the blood (FIG. 6g). Furthermore, we observed significantly decreased frequencies and numbers of Hmgb2^{-/-} P14 T cells in spleens at 20 dpi (FIG. 6h, i). Similar results were seen when exhausted WT and Hmgb2^{-/-} P14 T cells were transferred separately into WT hosts and re-challenged with LCMV Arm (FIG. 6j). These findings showed HMGB2 expression is essential for the re-expansion of exhausted CD8⁺ T cells after a secondary viral challenge.

### HMGB2 Regulates the Chromatin Accessibility of Tpex and Tex Genes During C113 Infection

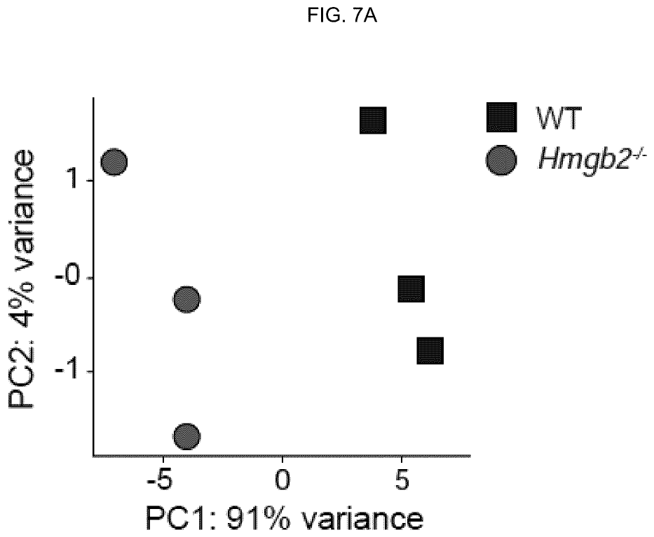

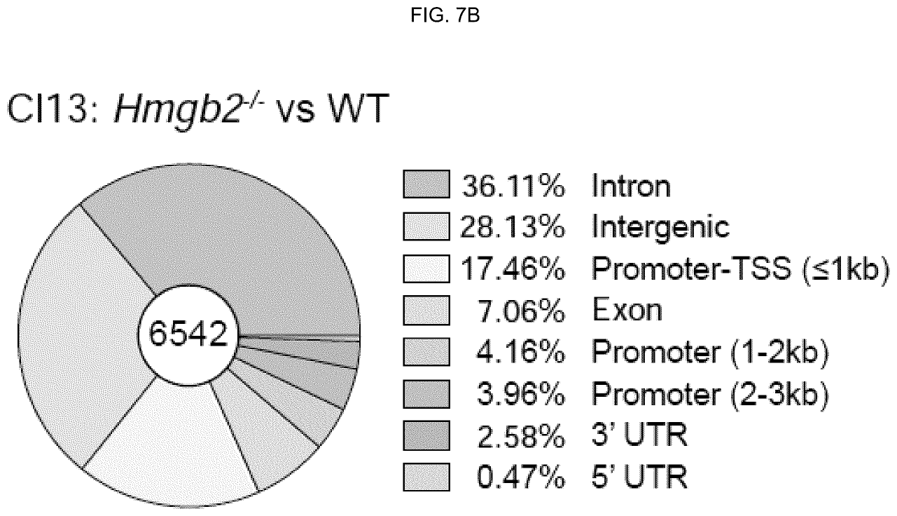

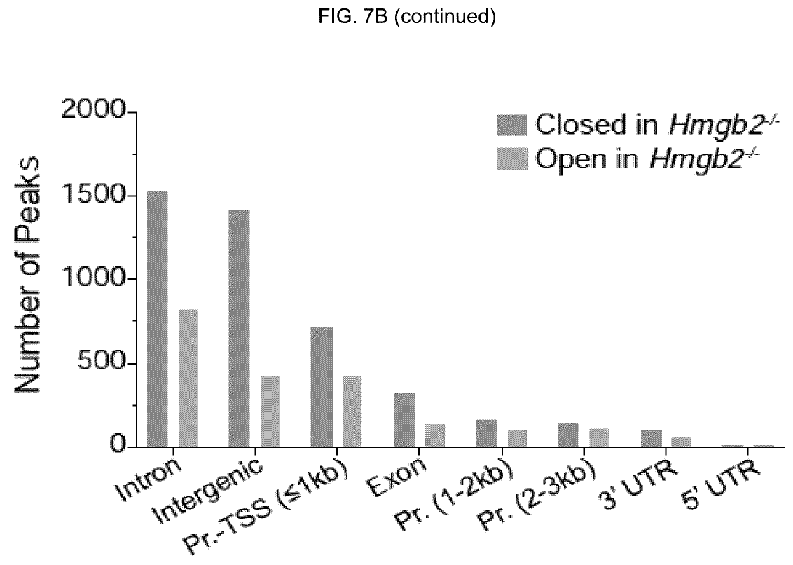

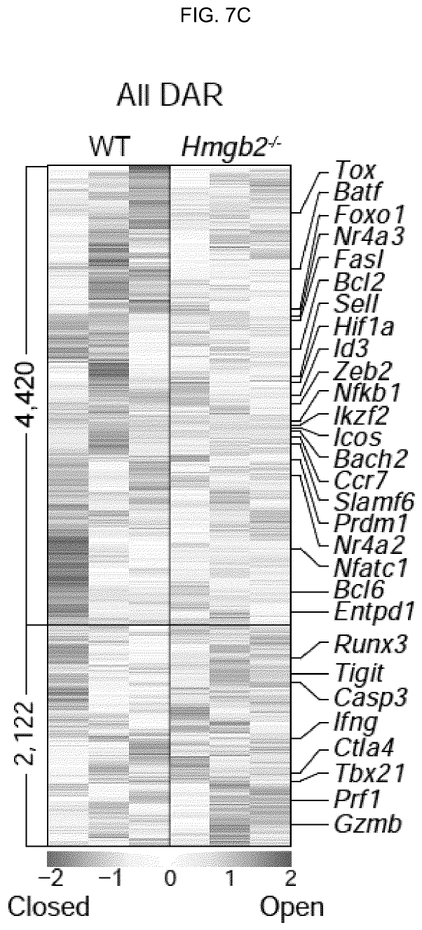

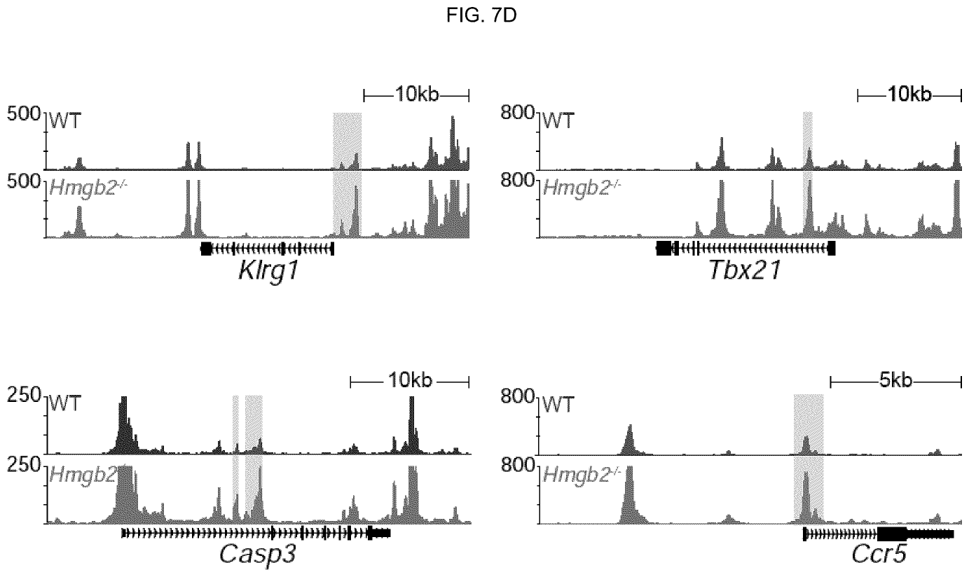

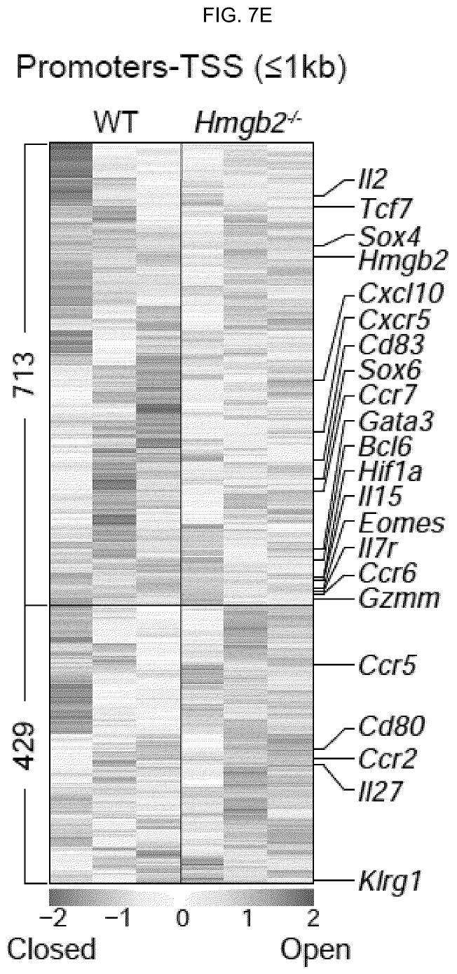

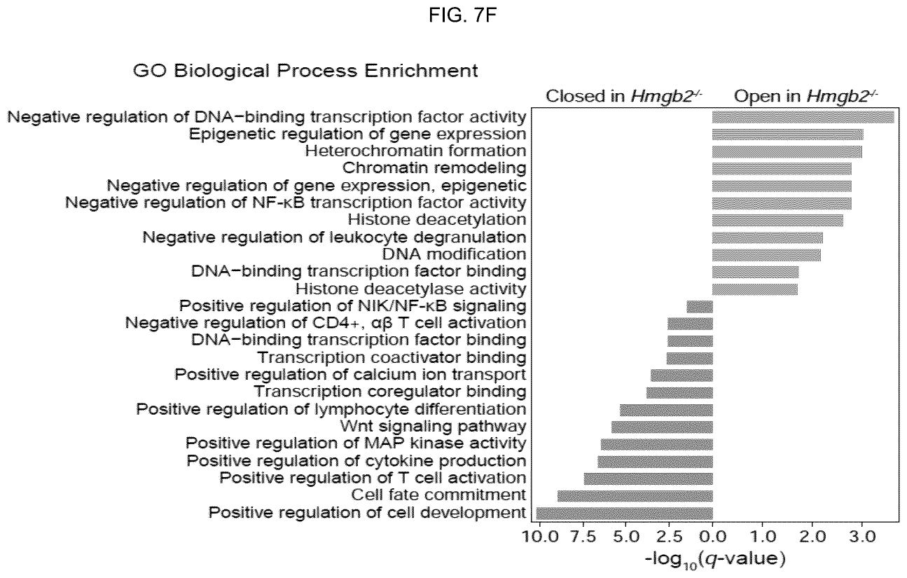

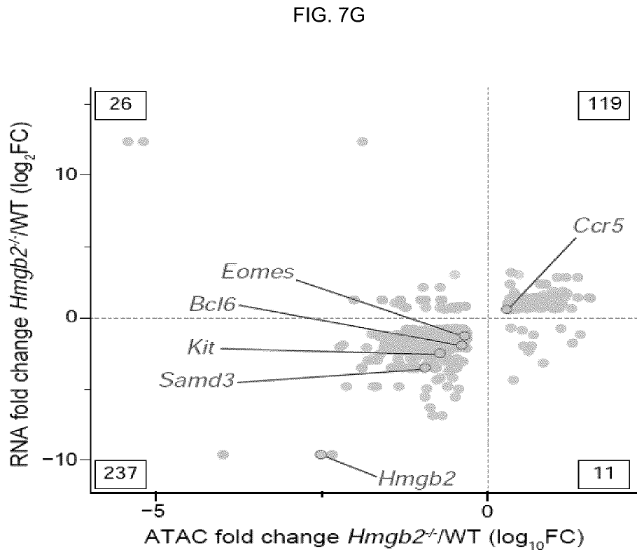

Since HMGB2 has a well-characterized role in chromatin remodeling, we next asked whether HMGB2 regulates the epigenetic program of exhausted T cells. We sorted WT and Hmgb2^{-/-} P14 T cells from mice at 8 dpi C113 and used ATAC-seq to identify significant changes in chromatin accessibility in the absence of HMGB2. PCA of the ATAC-seq profiles segregated WT and Hmgb2^{-/-} P14 T cells across PC1 (91% variance), indicating that Hmgb2 has a significant effect on chromatin accessibility (FIG. 7a). We found 6,542 differentially accessible regions (DAR), with most having decreased accessibility in Hmgb2^{-/-} P14 T cells compared with WT (FIG. 7b). Genomic annotation showed about 17.5% of these accessibility changes were at promoters (≤1 kb) or transcription start sites (TSS) (FIG. 7b). The genes in close proximity to loci with reduced accessibility in Hmgb2^{-/-} P14 T cells were associated with Tpex cells, including Batf Foxo1, Id3, Ikzf2, Slamf6, Sell and Bach2 (FIG. 7c)³⁹. In contrast, loci with increased accessibility were near genes associated with terminal Tex cells, including Tigit, Klrg1, Ccr5, Casp3, Ifng, Ctla4, Tbx21, Prf1, and Gzmb (FIG. 7c, d)³⁹. We observed a similar trend when looking at promoters-TSS (≤1 kb) containing DAR. Promoters of genes associated with Tpex cells had reduced accessibility in Hmgb2^{-/-} P14 T cells (Tcf7, Cxcr5, Ccr7, Gata3, Bcl6, Eomes, Il7r), while promoters of terminal Tex genes were more accessible (Klrg1, Ccr5) (FIG. 7e)^{10, 44, 45, 46}.

To further characterize genes associated with DAR at promoters-TSS (≤1 kb), we performed pathway enrichment. Genes with increased accessibility at promoters in Hmgb2^{-/-} P14 T cells showed significant enrichment for GO terms associated with (i) negative regulation of DNA-binding transcription factor activity; (ii) heterochromatin formation; and (iii) negative regulation of gene expression (FIG. 7f). Conversely, genes with less accessible promoter regions in Hmgb2^{-/-} P14 T cells had significant enrichment of GO terms associated with (i) positive regulation of cell development; (ii) positive regulation of T cell activation; and (iii) transcription coregulator/coactivator binding (FIG. 7f). To assess the correlation between chromatin accessibility and gene transcription, we compared these DAR with our 8 dpi C113 DEG (FIG. 4a, b). Overall, the epigenetic changes induced by HMGB2 corresponded to functionally relevant events, with the majority of DEG having accompanying changes in chromatin accessibility (119 upregulated DEG with increased accessibility, 237 downregulated DEG with decreased accessibility) (FIG. 7g). For instance, the promoters-TSS (≤1 kb) for Eomes, Bcl6 and Samd3 were less accessible with lower RNA expression in Hmgb2^{-/-} P14 T cells, while the Ccr5 promoter was more accessible with increased RNA expression in Hmgb2^{-/-} P14 T cells (FIG. 7g). Our data suggests a significant role for HMGB2 in CD8⁺ T cell chromatin accessibility, and more specifically, the opening of genomic regions associated with progenitor cells and the closing of genomic regions associated with terminal cell differentiation. Together, these data contribute to our understanding of the mechanisms by which HMGB2 regulates the development of exhausted CD8⁺ T cells.

### Cell-Intrinsic HMGB2 Expression in CD8⁺ T Cells is Required for Anti-Tumor Responses

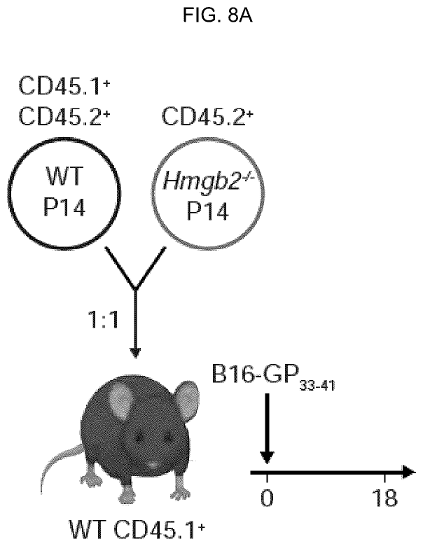

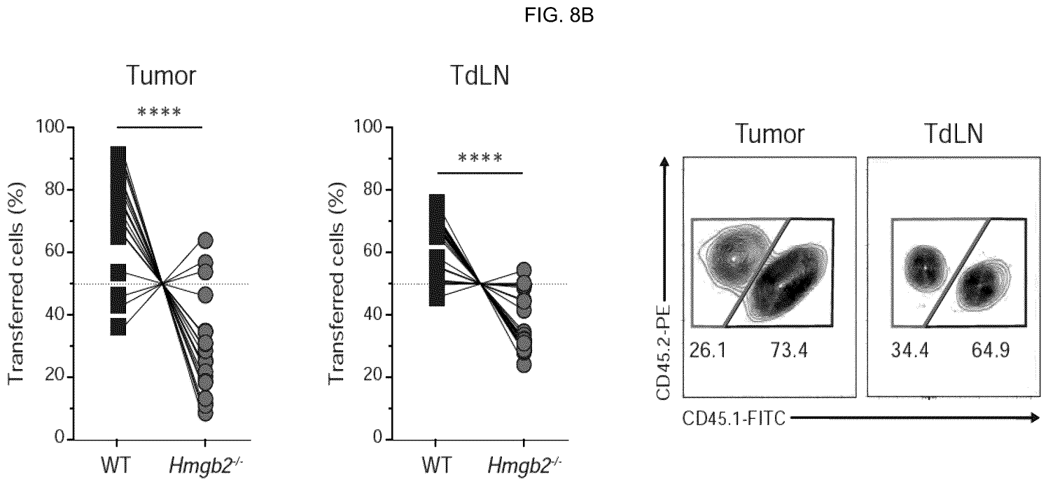

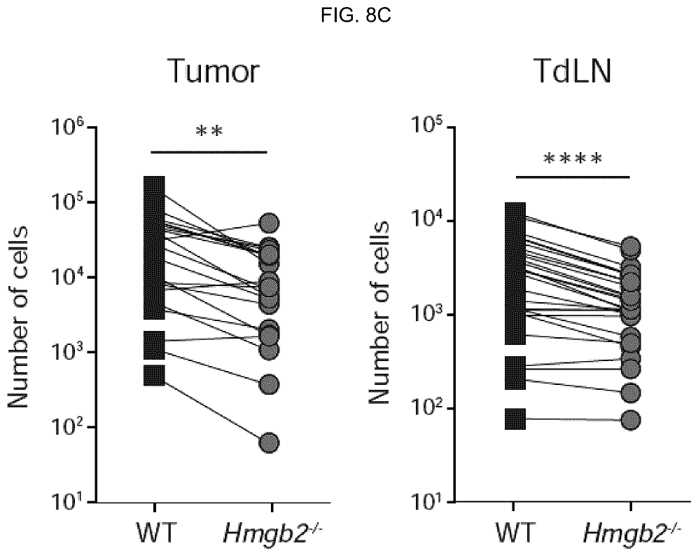

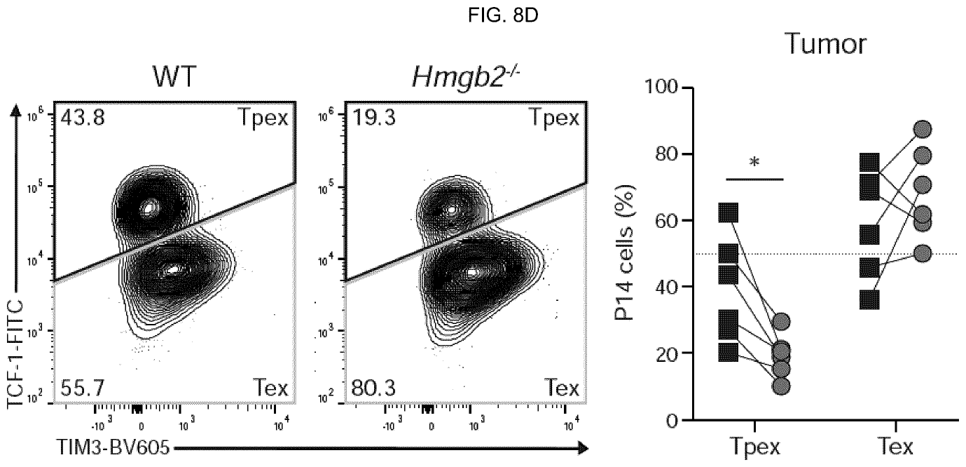

Since persistent antigen presentation in tumors also drives differentiation of exhausted CD8⁺ T cells, we next asked whether HMGB2 regulated tumor-specific CD8⁺ T cells. We co-transferred congenically marked (1×10⁶ cells/each) WT and Hmgb2^{-/-} P14 T cells at a 1:1 ratio into WT mice and subcutaneously injected B16-GP₃₃₋₄₁ melanoma cells (1×10⁶ cells) a day later (FIG. 8a). B16-GP₃₃₋₄₁ melanoma cells are highly aggressive and express the LCMV GP₃₃₋₄₁ epitope, which is recognized by P14 T cells. We observed significantly decreased frequencies of Hmgb2^{-/-} P14 T cells compared to WT in the tumor and tumor draining lymph nodes (TdLNs) at day 18 post melanoma cell injection (FIG. 8b). The numbers of Hmgb2^{-/-} P14 T cells were also significantly decreased in the tumor and TdLNs (FIG. 8c). We next evaluated the frequencies of Tpex and Tex cells by measuring TCF-1 and TIM-3 expression of tumoral WT and Hmgb2^{-/-} P14 T cells and found significantly less Hmgb2^{-/-} Tpex cells compared to WT, similar to our findings during C113 infection (FIG. 8d, 5f, S7a). These data indicate HMGB2 expression is also required for the maintenance and differentiation of Tpex and exhausted CD8⁺ T cells in melanoma tumors.

### DISCUSSION

Our findings showed a cell-intrinsic role for HMGB2 in the differentiation and stemness of memory and exhausted CD8⁺ T cells in viral and tumor models. We found HMGB2 expression and upregulation in effector, memory and exhausted CD8⁺ T cells. During acute viral infection, we found an important role for HMGB2 in memory T cell differentiation, memory precursor effector (MPEC) and central memory (Tcm) T cell phenotypes, and memory recall responses. In response to chronic viral infection, exhausted Hmgb2^{-/-} CD8⁺ T cells showed decreased progenitor exhausted T cell (Tpex) differentiation and survival, with these cells unable to persist during prolonged infection. Despite Hmgb2^{-/-} CD8⁺ T cells expressing TCF-1 and TOX master regulators, these transcription factors failed to induce the differentiation of Tpex and terminal exhausted T cells (Tex). Transcriptomic and chromatin accessibility analyses revealed that HMGB2 in exhausted CD8⁺ T cells functioned to increase expression and accessibility of Tpex-specific gene signatures, while decreasing expression and chromatin accessibility of Tex gene signatures.

After acute viral infections, effector CD8⁺ T cells differentiate into memory T cells (Tmem), which self-renew, persist long term, and provide protection upon secondary infection with the same pathogen^{47, 48, 49, 50}. We found that HMGB2 regulated memory CD8⁺ T cell differentiation, as shown by the increased MPEC phenotype in Hmgb2^{-/-} CD8⁺ T cells. Interestingly however, we observed increased apoptosis in Hmgb2^{-/-} MPECs, indicating that HMGB2 promoted the survival of this population. We also found decreased Hmgb2^{-/-} Tcm cells. Tcm cells are important for Tmem responses as they contribute to their proliferation, longevity, multipotency and recall potential^{51, 52}. We correspondingly found diminished maintenance and recall capacity of Hmgb2^{-/-} Tmem cells, which could be attributed to this decrease in Tcm phenotype. Additionally, although Hmgb2^{-/-} Tmem cells had high expression of the transcription factor TCF-1, a critical regulator for memory T cell transcriptional programs and Tcm-mediated recall responses^{43, 53, 54}, they were still unable to persist and respond to secondary infection. Our findings highlight an important function of HMGB2 in the differentiation, survival, and recall function of memory CD8⁺ T cells.

Compared to effector and memory T cells, the regulation of exhausted CD8⁺ T cells remains poorly understood. Persistent C113 infection induces NFAT and calcineurin signaling, which induces TOX expression in CD8⁺ T cells^{36, 38, 55}. We observed similar levels of TOX expressed in WT and Hmgb2^{-/-} CD8⁺ T cells during C113 infection, suggesting effective TCR, NFAT, and calcineurin signaling in Hmgb2-CD8⁺ T cells. Accordingly, both WT and Hmgb2^{-/-} P14 T cells expanded to similar numbers by 8 dpi C113 infection, indicating that Hmgb2^{-/-} P14 T cells were effectively primed and activated during early stages of chronic viral infection. However, despite similar phenotypes and initial responses of exhausted WT and Hmgb2^{-/-} P14 T cells, Hmgb2^{-/-} P14 T cells drastically declined after 8 dpi C113 infection and did not persist. During C113 infection, it has been shown that the transcription factors TOX and TCF-1 are critical to form exhausted T cells^{2, 37}. However, the loss of exhausted Hmgb2^{-/-} P14 T cells was not due to diminished TOX or TCF-1 expression, as both transcriptional regulators were expressed at similar levels to WT. Importantly, TOX and TCF-1 were insufficient to establish and sustain Hmgb2^{-/-} Tex cells throughout C113 infection. This links HMGB2 proteins with TOX and TCF-1 regulation of exhausted T cell differentiation.

HMGB2 proteins regulate cellular stemness, as shown by defects in various differentiation programs in Hmgb2^{-/-} mice^{56, 57, 58, 59, 60}. During chronic infection, stem-like Tpex cells arise, which can self-renew and seed the Tex pool. Using transcriptomics, we found HMGB2 positively regulated the expression of Tpex cell associated genes and correspondingly, we found HMGB2 positively regulated Tpex cell frequencies, numbers, and survival. HMGB2's regulation of Tpex cell differentiation and long-term maintenance is similar to that of TCF-1, a key transcriptional regulator of Tpex-specific programming; Tcf7^{-/-} CD8⁺ T cells fail to develop into Tpex cells and decline throughout C113 infection^{2, 61}. However, despite the loss of Hmgb2^{-/-} Tpex cells, Hmgb2^{-/-} T cells had similar frequencies of TCF-1⁺ cells to WT, suggesting HMGB2 and TCF-1 co-regulate Tpex differentiation. There is significant clinical interest in understanding the differentiation of Tpex cells for immunotherapy use in cancer and chronically infected patients; Tpex cells provide the effector T cell proliferative burst after immune checkpoint blockade (ICB) and may have therapeutic predictive value in patients^{62, 63, 10, 15, 16}. Although we did not evaluate the response of Hmgb2^{-/-} Tpex cells to anti-PD-1/anti-PD-L1 blockade, we observed a defect in their reinvigoration with secondary Arm infection. Therefore, combining HMGB2 modulation with anti-PD-1/anti-PD-L1 therapy may enhance and preserve Tpex differentiation and increase clinical efficacy in the settings of chronic infections and cancer. Together, our findings showed HMGB2 regulates the differentiation, survival, and reinvigoration of Tpex cells, and may help predict ICB efficacy.

Exhausted T cells develop permanent epigenetic marks early in their differentiation, with additional epigenetic changes acquired at later stages of exhaustion^{4, 5, 8}. Since the epigenetic program of exhausted T cells is relatively stable, ICBs can only transiently reinvigorate exhausted T cells, as they reacquire their exhausted epigenetic program over time^{11, 18, 21, 22, 23}. Combining chromatin remodeling with ICBs may represent a new clinical approach to increase the reinvigoration potential of exhausted T cells. Therefore, identifying exhaustion-specific epigenetic regulators is a pressing clinical need for patients with chronic diseases. HMGB2 is a known chromatin modifier, but its role in the chromatin remodeling and epigenetic programming of exhausted T cells is not known. Here, we found that HMGB2 regulated the accessibility of genomic regions in exhausted T cells, with most of these changes corresponding to functionally relevant events in gene transcription. HMGB2 directly supported the accessibility of Tpex associated genes while decreasing the accessibility of Tex associated genes. Since many loci regulated by HMGB2 are also known transcriptional targets of TCF-1, we hypothesize that HMGB2 modulates epigenetic accessibility and thus gene expression of TCF-1 targets and potentially other transcriptional regulators which promote Tpex cell differentiation and maintenance. Notably, Ingenuity Pathway Analysis (IPA) identified the TCF-1 master regulatory pathway as being significantly inhibited in Hmgb2^{-/-} P14 T cells, suggesting HMGB2 is required for TCF-1 mediated transcriptional programs in exhausted T cells. HMGB2 may enhance TCF-1 binding to its targets, as it does with LEF-1, a transcription factor functionally similar to TCF-1^{59, 65}. Furthermore, TCF-1 and LEF1 have been shown to regulate CD8⁺ T cell identity and function, with ablation of these transcription factors resulting in expression of non-T cell lineage genes⁶⁶. Although exhausted Hmgb2^{-/-} P14 T cells had high TCF-1 expression, we found similar aberrant T cell gene expression at 20 dpi C113 (Cd19, Cd4, and immunoglobulins). We propose that HMGB2 and TCF-1 co-regulate exhaustion-specific transcriptional and epigenetic programs in CD8⁺ T cells through chromatin remodeling and facilitating transcription factor binding.

In summary, we show that Hmgb2 expression is required for the differentiation and survival of memory and Tpex cells during acute and chronic viral infection, respectively. We detected HMGB2 expression in naïve, effector, memory, and exhausted T cells after LCMV infection in mice, with higher levels during persistent infection. Similarly, patients with chronic hepatitis C infection had higher HMGB2 levels in exhausted CD8⁺ T cells which decreased after patients were cured. In the setting of acute viral infection, Hmgb2^{-/-} memory CD8⁺ T cells developed, but they were defective in their differentiation and recall capacity. During chronic C113 infection, Hmgb2^{-/-} CD8⁺ T cells initially proliferated and expanded to similar levels as WT but were severely hindered in their formation of Tpex cells, thus preventing long-term exhausted T cell responses. We found HMGB2 increased the accessibility of Tpex signature genes, positively regulating the transcriptional program, differentiation, and maintenance of Tpex cells. We also observed decreased Hmgb2^{-/-} Tpex cells in melanoma tumors and tumor-draining lymph nodes, indicating HMGB2 sustains exhausted T cells in multiple models of persistent antigen. We found a novel and previously unidentified role for HMGB2 in the differentiation and survival of functional memory and exhausted T cells, with vast implications for secondary reinfections and immunotherapies to cancer and chronic viruses. This new understanding of HMGB2's role in exhausted T cell stemness is a novel contribution to TCF-1 and TOX mediated regulation of exhaustion and shows that HMGB2 is an indispensable partner of TCF-1 and TOX in the formation and maintenance of exhausted T cells.

### FIGURE LEGENDS

FIG. 1. HMGB2 expression in murine and human virus-specific CD8⁺ T cells.

Expression levels of HMGB2 in GP₃₃₋₄₁⁺ CD8⁺ T cells (a) and GP₂₇₆₋₂₈₆⁺ CD8⁺ T cells (b) assessed by flow cytometry in spleen with n=4 mice. Naïve, uninfected; Effector, 8 dpi LCMV Arm; Memory, 30 dpi LCMV Arm; Early Exhaustion, 8 dpi LCMV C113; Late Exhaustion, 30 dpi LCMV C113; MFI, mean fluorescence intensity. Data is mean±s.e.m and representative of two independent experiments with ≥4 mice per group. (c) Representative IMAGESTREAM™ analysis of GP₃₃₋₄₁⁺ CD8⁺ T cells, magnification, 60×. Data are representative of two independent experiments with ≥4 mice per group. For d-e, low-input RNA-sequencing data from Hensel, N. et al., 2021 (GSE150345)²². (d) Average normalized HMGB2 expression in human HCV-specific CD8⁺ T cells, isolated from three patients during chronic Hepatitis C (cHCV) infection and after direct-acting antiviral (DAA) cure. Significance assessed by differential analysis with multi-factor design of paired samples using DESeq2. (e) Average FPKM fold change of exhaustion associated genes after cHCV cure in patients from d. **p≤0.01, ***p≤0.001, ****p≤0.0001, one-way ANOVA (analysis of variance) (a-b), paired Student's t-test (d).

FIG. 2. Cell-intrinsic kinetics of WT and Hmgb2^{-/-} P14 T cells during Arm infection.

(a) Experimental scheme for b-d. WT and Hmgb2^{-/-} P14 CD8⁺ T cells were transferred separately into naïve mice and infected with LCMV Arm. Blood taken at 8, 15, 26, and 35 dpi. Spleens isolated at 68 dpi. (b) Frequency of WT and Hmgb2^{-/-} P14 T cells of total CD8⁺ population. (c) Splenic WT and Hmgb2^{-/-} P14 T cell frequencies and numbers at 68 dpi. (d) Cytokine production by splenic WT and Hmgb2^{-/-} P14 T cells at 68 dpi. (e) Experimental scheme for f-j. WT and Hmgb2^{-/-} P14 T cells were co-transferred at 1:1 into WT mice and infected with LCMV Arm. Frequencies (f) and numbers (g) of splenic WT and Hmgb2^{-/-} P14 T cells at indicated timepoints post infection. Frequencies of splenic WT and Hmgb2^{-/-} P14 short-lived effector (SLEC) and memory precursor effector (MPEC) T cells in the blood at 8 dpi (h) and 46 dpi (i) Arm. (j) Frequencies of WT and Hmgb2^{-/-} P14 central memory (Tcm), effector memory (Tem) and terminal Tem (t-Tem) T cells at 46 dpi Arm in the spleen. Data are representative of three independent experiments with ≥5 mice per group. Data is mean±s.e.m. *p≤0.05, **p≤0.01, ****p≤0.0001, two-tailed unpaired Student's t-test (b-d), paired Student's t-test (f-j).

FIG. 3. Cell-intrinsic kinetics of WT and Hmgb2^{-/-} P14 T cells during C113 infection.

(a) Experimental scheme for b-d. WT and Hmgb2^{-/-} P14 CD8⁺ T cells were transferred separately into naïve mice and infected with LCMV C113. Blood taken at 8, 15, 26, and 35 dpi. Spleens isolated at 68 dpi. (b) Frequency of WT and Hmgb2^{-/-} P14 T cells of total CD8⁺ population. (c) Splenic WT and Hmgb2^{-/-} P14 T cell frequencies and numbers at 68 dpi C113. (d) Cytokine production by splenic WT and Hmgb2^{-/-} P14 T cells at 68 dpi C113. (e) Experimental scheme for f-g. WT and Hmgb2^{-/-} P14 T cells were co-transferred at 1:1 into WT mice and infected with C113. Frequencies (f) and numbers (g) of splenic WT and Hmgb2^{-/-} P14 T cells at indicated timepoints post infection. Data are representative of three independent experiments with ≥5 mice per group. (h) Frequencies of total CD8⁺, GP₃₃₋₄₁⁺, GP₂₇₆₋₂₈₆⁺, and NP₃₉₆₋₄₀₄⁺ T cells during C113 infection in the blood of WT and Hmgb2^{-/-} mice. (i) Frequencies of total CD8⁺, GP₃₃₋₄₁⁺, GP₂₇₆₋₂₈₆⁺, and NP₃₉₆₋₄₀₄⁺ T cells during C113 infection in the spleen and lymph nodes (LNs) of WT and Hmgb2^{-/-} mice at 44 dpi. Virus titers at 44 dpi measured from sera (j) and kidneys (k) by plaque forming units (PFU) and expressed as PFU/mL or PFU/g, respectively. Data are representative of two independent experiments with ≥3 mice per group. Data is mean±s.e.m. *p≤0.05, **p≤0.01, ***p≤0.001, ****p≤0.0001, two-tailed unpaired Student's t-test (b-d, h-k), paired Student's t-test (f-g).

FIG. 4. Hmgb2^{-/-} CD8⁺ T cells have decreased expression of Tpex signature genes.

Bulk RNA-seq analysis of WT and Hmgb2^{-/-} P14 T cells during LCMV Arm and C113 infection. (a) Principal component analysis (PCA) of WT and Hmgb2^{-/-} P14 T cells at 8 days post either Arm or C113 infection. (b) Volcano plot highlighting differentially expressed genes (DEG) between WT and Hmgb2^{-/-} P14 T cells at 8 dpi C113. Significant DEG (padj≤0.1, |log₂FC|≥0.5) are colored (pink=upregulated in Hmgb2^{-/-} P14 T cells; black=upregulated in WT P14 T cells). (c) Left: Gene ontology (GO) biological process enrichment from Metascape of significant DEG from b. X-axis represents log₁₀(q-value) and size of dot represents proportion of the total DEG enriched to that given pathway. Right: Heatmap of average normalized expression of genes associated with bolded pathways. Each column represents one independent experiment with n=5 mice. (d) PCA of co-transferred WT and Hmgb2^{-/-} P14 T cells at 20 dpi C113. (e) Volcano plot highlighting DEG between WT and Hmgb2^{-/-} P14 T cells at 20 dpi C113. Significant DEG (padj≤0.1, |log₂FC|≥0.5) are colored (pink=upregulated in Hmgb2^{-/-} P14 T cells; black=upregulated in WT P14 T cells). (f) Left: GO biological process enrichment from Metascape of DEG from e. X-axis represents log₁₀(q-value) and size of dot represents proportion of the total DEG enriched to that given pathway. Right: Heatmap of average normalized expression of genes associated with bolded pathways. Each column represents one independent experiment with n=10 mice.

FIG. 5. Hmgb2^{-/-} CD8⁺ T cells have decreased progenitor exhausted T cell differentiation

WT and Hmgb2^{-/-} P14 T cells were co-transferred at 1:1 into WT mice, followed by LCMV Arm or C113 infection. (a) BrdU uptake of splenic WT and Hmgb2^{-/-} P14 T cells at 8 dpi. Caspase3 and PI staining of P14 T cells at 8 dpi (b) and 46 dpi (c) in the spleen. (d) Alkaline comet assay of splenic WT and Hmgb2^{-/-} P14 T cells isolated on 8 days post either Arm or C113 infection (pooled samples from 10 mice/group). Representative fluorescent comet images of cells stained with Vista Green DNA dye. U2OS human cells treated with etoposide (10 μM for 30 min), a topoisomerase II inhibitor used to generate DNA double-strand breaks in cells, served as controls for comet tail formation. (e) p-H2AX (Ser139) protein expression by western blot in purified splenic WT and Hmgb2^{-/-} P14 T cells isolated on 8 days post either Arm or C113 infection (pooled samples from 10 mice/group). U2OS human cells untreated or treated with 25 μM etoposide for 60 min served as negative and positive controls, respectively. (f) Frequencies and numbers of splenic progenitor exhausted (Tpex) and terminal exhausted (Tex) T cells at 8 dpi C113. (g) Frequencies of CXCR5′ P14 T cells at 8 dpi C113 in the spleen. Data are representative of three independent experiments with ≥5 mice per group. Data is mean±s.e.m. *p≤0.05, **p≤0.01, ****p≤0.0001, paired Student's t-test.

FIG. 6. Memory and exhausted Hmgb2^{-/-} CD8⁺ T cells are defective in their recall capacity.

(a) Experimental scheme for b-d. WT and Hmgb2^{-/-} P14 T cells were co-transferred into WT mice at 1:1, followed by LCMV Arm infection. At 30 dpi, memory WT and Hmgb2^{-/-} P14 T cells were sorted and normalized to 1:1 before co-transferred into new naïve mice, followed by Arm infection (secondary infection). (b) Frequency of WT and Hmgb2^{-/-} P14 T cells in blood during secondary Arm infection. Frequency (c) and number (d) of splenic WT and Hmgb2^{-/-} P14 T cells at 20 dpi secondary Arm. (e) WT and Hmgb2^{-/-} P14 T cells were transferred separately into WT hosts and sorted at 68 dpi Arm before adoptive transfer into separate naïve mice, followed by Arm infection (secondary infection). Frequency of WT and Hmgb2^{-/-} P14 T cells in the blood during secondary Arm infection. (f) Experimental scheme for g-i. WT and Hmgb2^{-/-} P14 T cells were co-transferred into WT mice at 1:1, followed by LCMV C113 infection. At 30 dpi, exhausted WT and Hmgb2^{-/-} P14 T cells were sorted and normalized to 1:1 before co-transferred into new naïve mice, followed by Arm infection (secondary infection). (g) Frequency of WT and Hmgb2^{-/-} P14 T cells in blood during secondary Arm infection. Frequency (h) and number (i) of splenic WT and Hmgb2^{-/-} P14 T cells at 20 dpi secondary Arm. (j) WT and Hmgb2^{-/-} P14 T cells were transferred separately into WT hosts and sorted at 68 dpi Arm before adoptive transfer into separate naïve mice, followed by Arm infection (secondary infection). Frequency of WT and Hmgb2^{-/-} P14 T cells in the blood during secondary Arm infection. Data are representative of three independent experiments with ≥10 mice per group. Data is mean±s.e.m. *p≤0.05, **p≤0.01, ****p≤0.0001, paired Student's t-test (b-d, g-i), two-tailed unpaired Student's t-test (e, j).

FIG. 7. Epigenetic programming of exhausted T cells by HMGB2

ATAC-seq analysis of WT and Hmgb2^{-/-} P14 T cells at 8 dpi LCMV C113. (a) Principal component analysis (PCA) of all samples by global chromatin accessibility profile. (b) Location of significantly differentially accessible ATAC-seq peaks (FDR≤0.05, |log₁₀FC|≥0.3). (c) Heatmap of all significantly differentially accessible loci (DAR). Numbers on left denote number of DAR. Each column represents a biological replicate of n=10 mice pooled. (d) ATAC-seq tracks of genes associated with effector (Teff) and terminal exhausted (Tex) T cells. DAR are highlighted with grey bars. (e) Heatmap of DAR within promoters-TSS (≤1 kb). Each column represents a biological replicate of n=10 mice pooled. (f) Gene ontology (GO) biological process enrichment from Metascape of DAR within promoters-TSS (≤1 kb) from e. (g) Fold change in ATAC accessibility versus RNA expression. Key genes with DAR in promoters-TSS (≤1 kb) are highlighted in red. Inset, table enumerating number of ATAC peak-gene pairs in each quadrant.

FIG. 8. HMGB2 regulation of anti-tumor CD8⁺ T cells.

(a) Experimental scheme for b-d. WT and Hmgb2^{-/-} P14 T cells were co-transferred into WT mice at 1:1 and given B16-GP₃₃₋₄₁ melanoma cells s.c. Tumors and tumor draining lymph nodes (TdLN) isolated at 18 dpi. Frequencies (b) and numbers (c) of WT and Hmgb2^{-/-} P14 T cells within the tumor and TdLN at 18 dpi. (d) Frequencies of progenitor exhausted (Tpex) and terminal exhausted (Tex) T cells at 18 dpi in the tumor. Data are representative of three independent experiments with ≥10 mice per group. Data is mean s.e.m. *p≤0.05, **p≤0.01, ****p≤0.0001, paired Student's t-test.

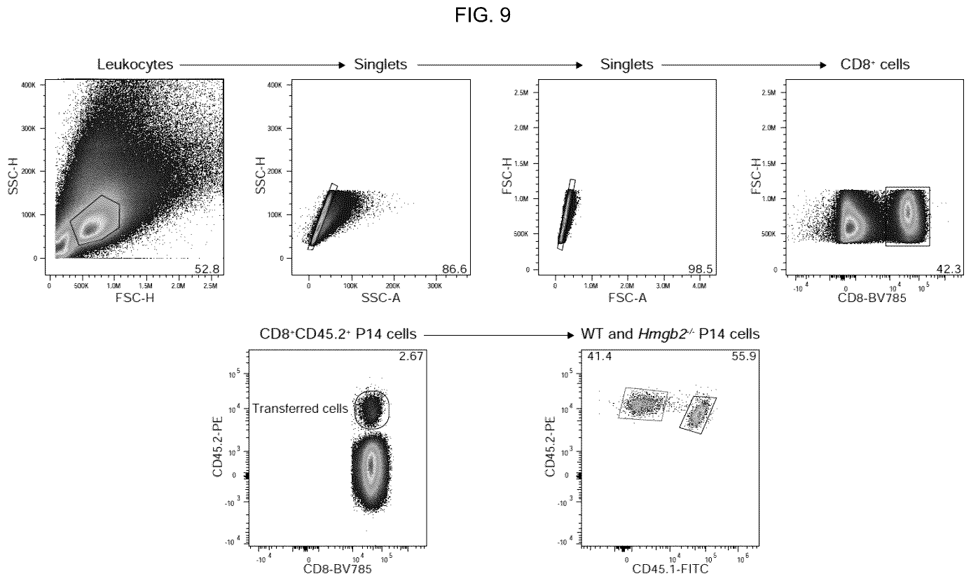

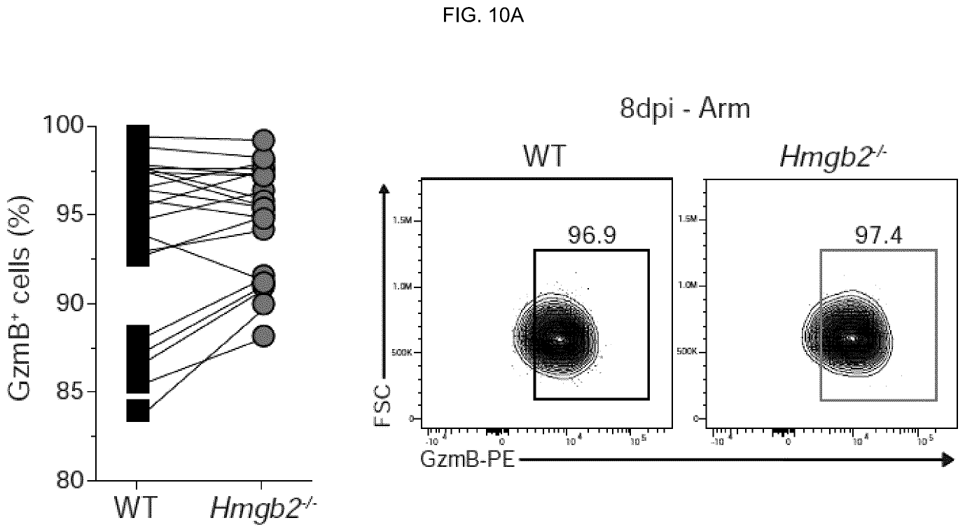

FIG. 9, or Supplementary FIG. 1. Gating strategy used in co-adoptive transfer experiments is illustrated.

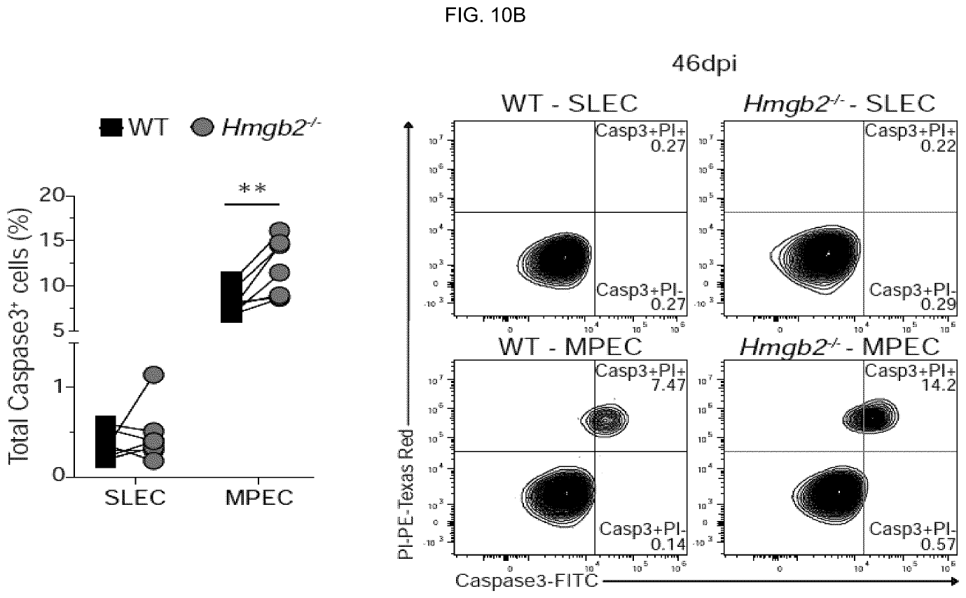

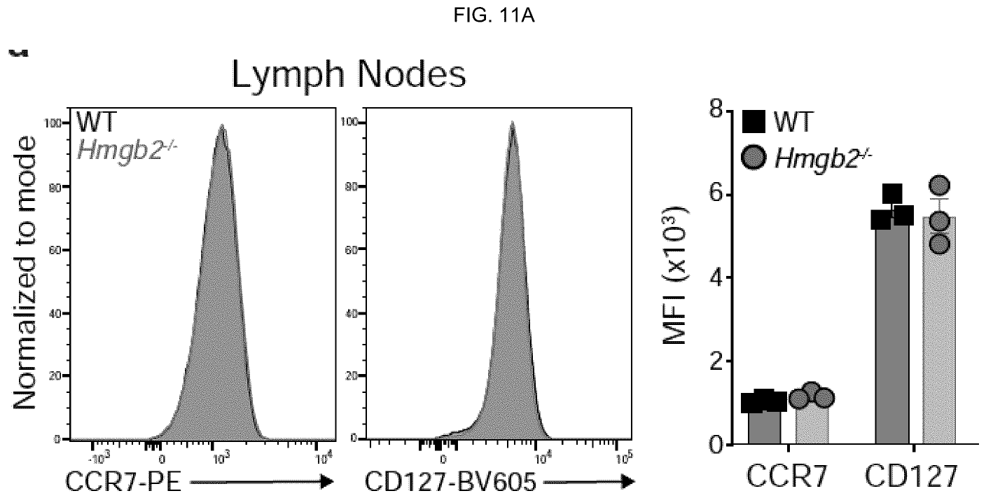

FIG. 10, or Supplementary FIG. 2. Effector function and survival of Hmgb2^{-/-} CD8⁺ T cells: Frequencies of GranzymeB⁺ WT and Hmgb2^{-/-} P14 T cells at 8 dpi LCMV Arm (a) frequency of total Caspase3⁺ splenic WT and Hmgb2^{-/-} short-lived effector (SLEC) and memory precursor effector (MPEC) P14 T cells at 46 dpi Arm. Each symbol represents an individual mouse. Data are representative of three independent experiments with ≥10 mice per group. Data is mean±s.e.m. *p≤0.05, **p≤0.01, Paired Student's t-test (a-c), two-sided Student's t-test (d).

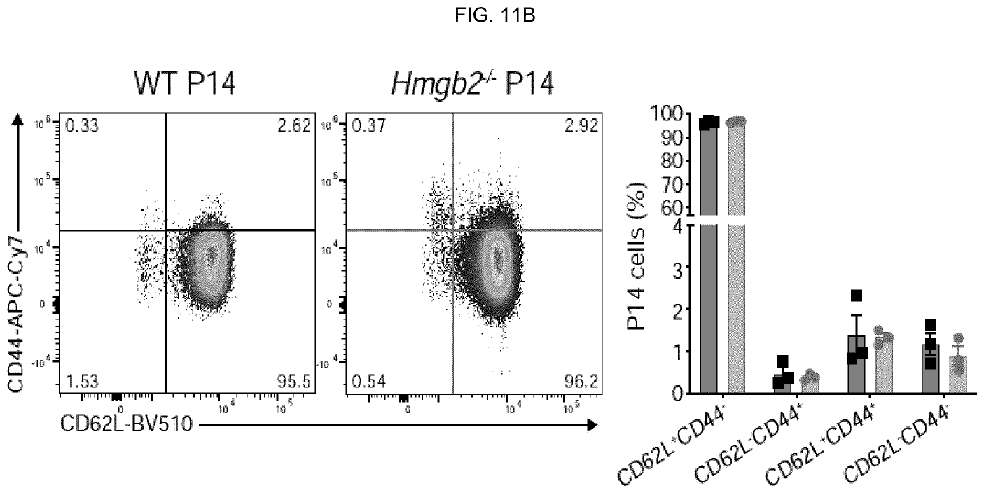

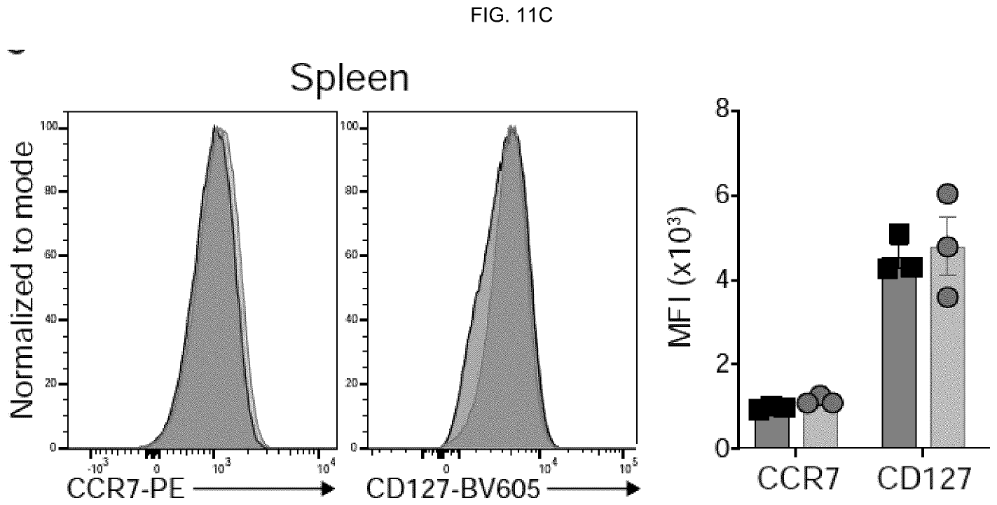

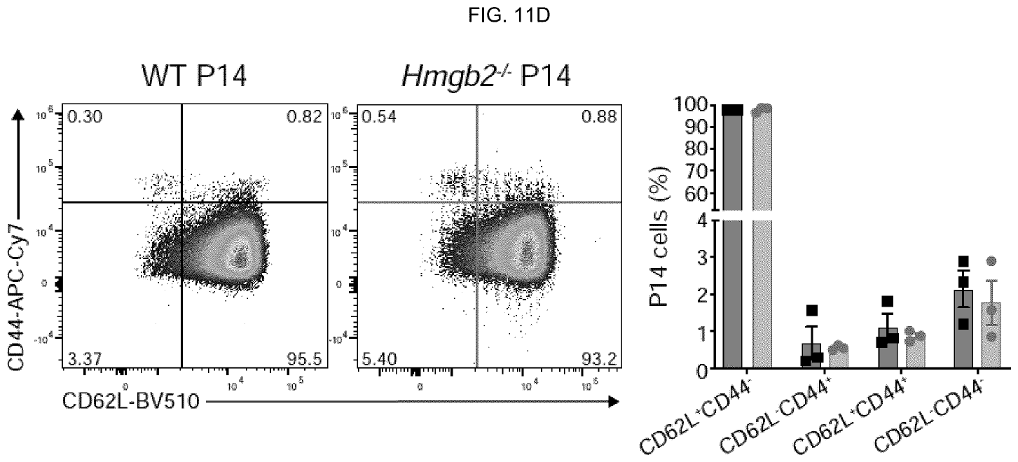

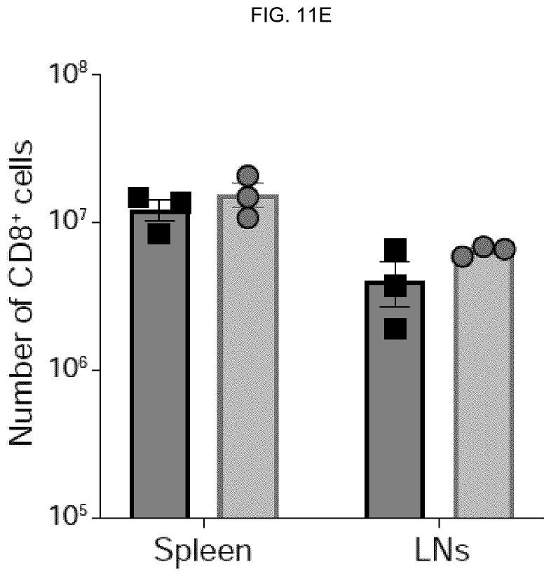

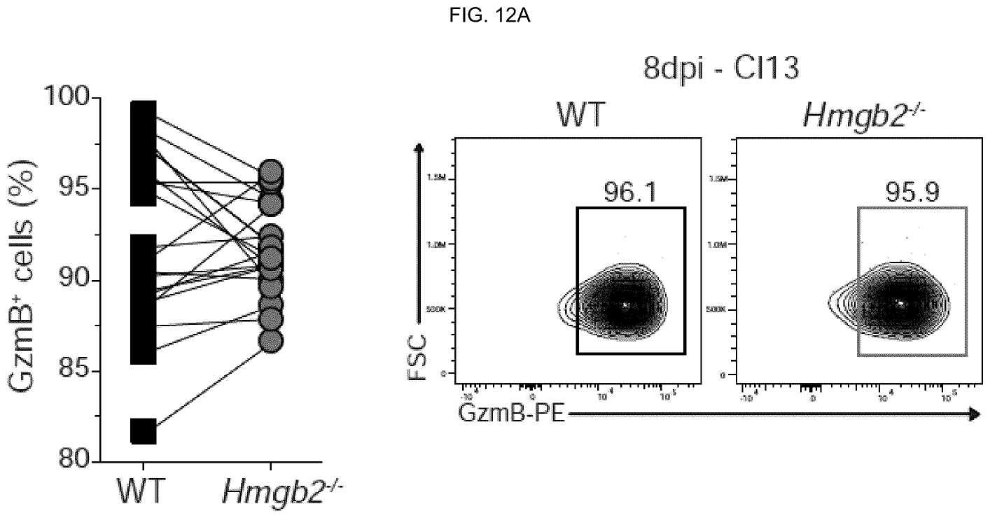

FIG. 11, or Supplementary FIG. 3. HMGB2 expression is dispensable for naïve CD8⁺ T cell development: CCR7/CD127 (a) and CD44/CD62L (b) protein expression of naïve WT and Hmgb2^{-/-} P14 T cells isolated from lymph nodes of uninfected mice. CCR7/CD127 (c) and CD44/CD62L (d) protein expression of naïve WT and Hmgb2^{-/-} P14 T cells isolated from spleens of uninfected mice. (e) Number of naïve WT and Hmgb2^{-/-} CD8⁺ T cells. Each symbol represents an individual mouse. Data are representative of two independent experiments with ≥5 mice per group. Data is mean±s.e.m. Two-sided Student's t-test.

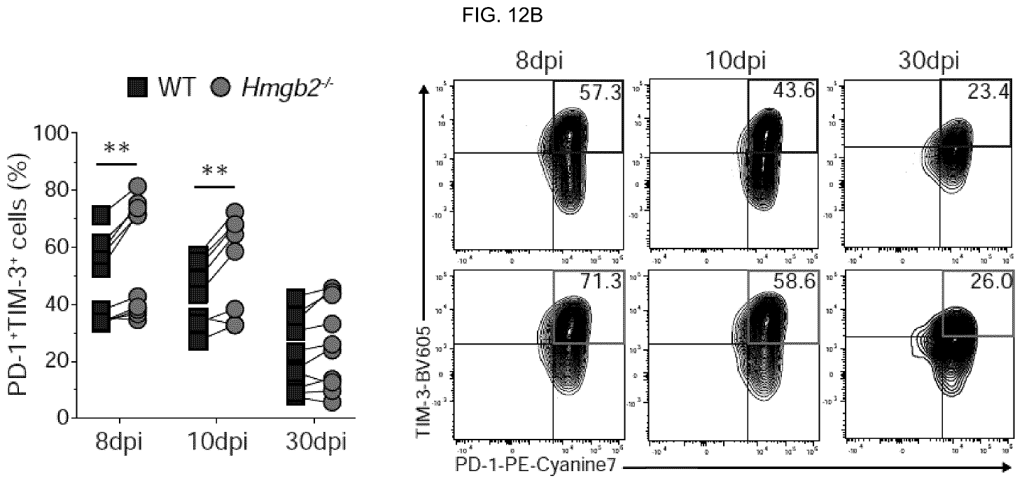

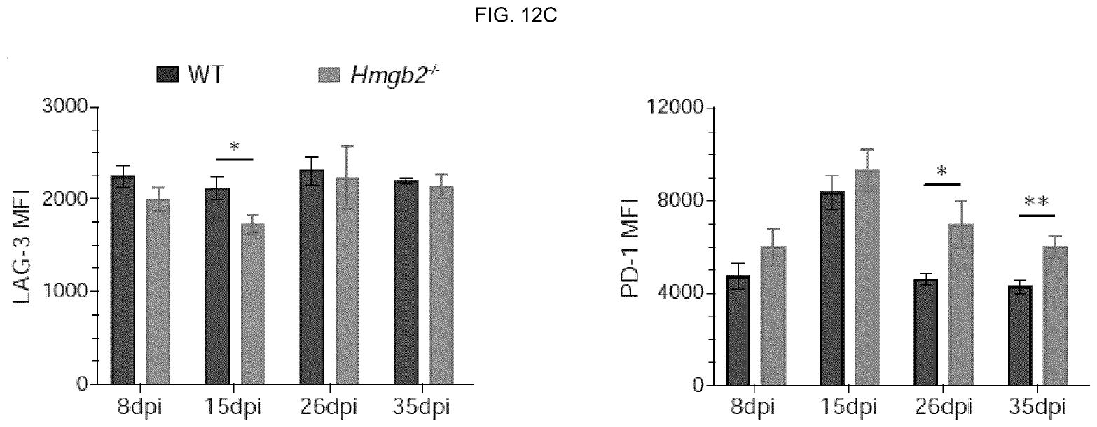

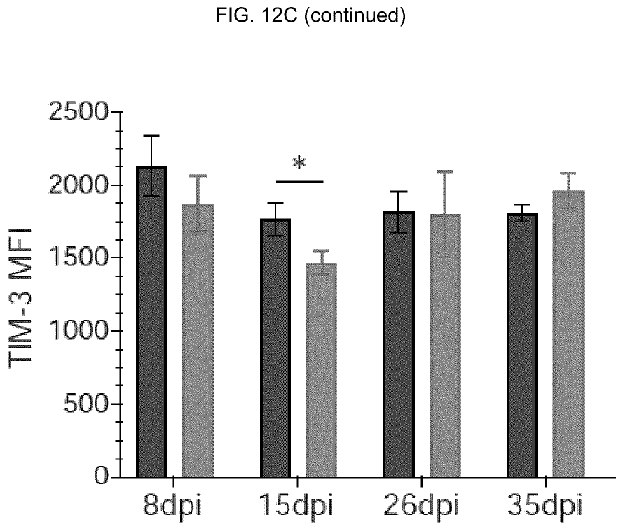

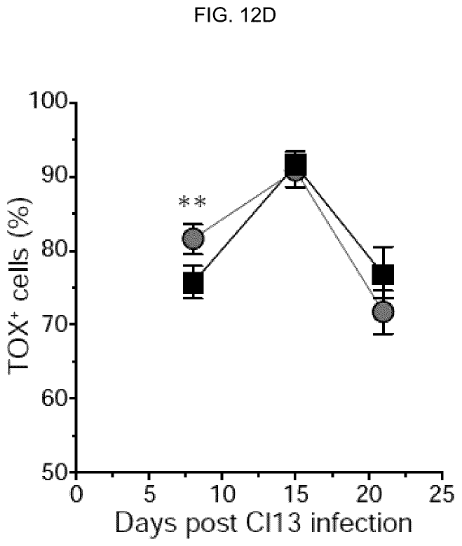

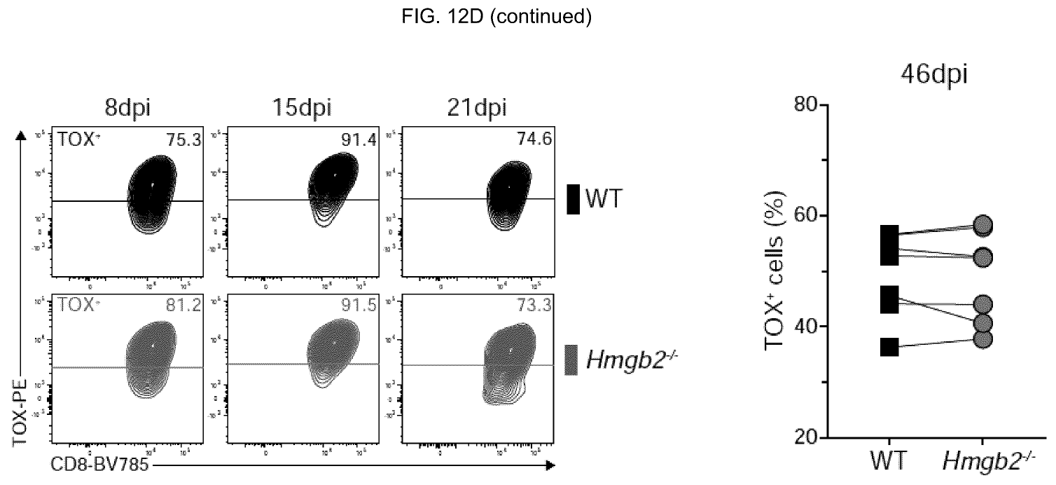

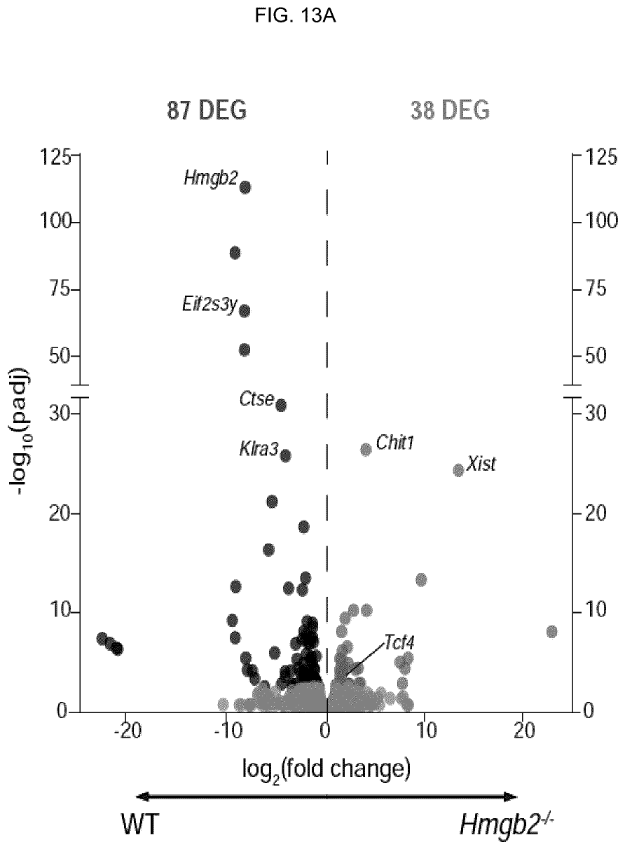

FIG. 12, or Supplementary FIG. 4. Effector function and Apoptosis of WT and Hmgb2^{-/-} P14 SLEC/MPEC and Tpex/Tex cells: WT and Hmgb2^{-/-} P14 T cells were co-transferred into mice followed by C113 infection. Frequencies of GranzymeB⁺ WT and Hmgb2^{-/-} P14 T cells at 8 dpi LCMV C113 (a) Frequencies of PD-1⁺TIM3⁺ T cells (b) Mean fluorescence expression of LAG-3, PD-1, and TIM-3 in CD8⁺ T cells after C113 infection (c). TOX expression at various times post-infection in WT Hmgb2^{-/-} P14 T cells (d). Each symbol represents an individual mouse. Data are representative of two independent experiments with ≥5 mice per group. Data is mean±s.e.m. **p≤0.01, ***p≤0.001, paired Student's t-test.

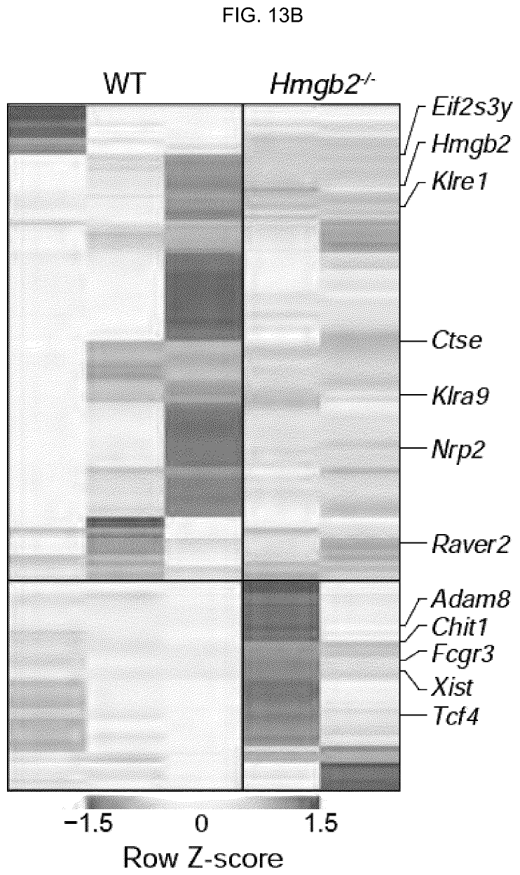

FIG. 13, or Supplementary FIG. 6: Transcriptomics of WT and Hmgb2^{-/-} CD8⁺ T cells after acute viral infection: (a) Volcano plot highlighting differentially expressed genes (DEG) between WT and Hmgb2^{-/-} P14 T cells at 8 dpi LCMV Arm infection from FIG. 4a. Significant DEG (padj≤0.1) are colored (pink=upregulated in Hmgb2^{-/-} P14 T cells; black=upregulated in WT P14 T cells). (b) Heatmap of average normalized expression of significant DEG. Each column represents one independent experiment with n=5 mice.

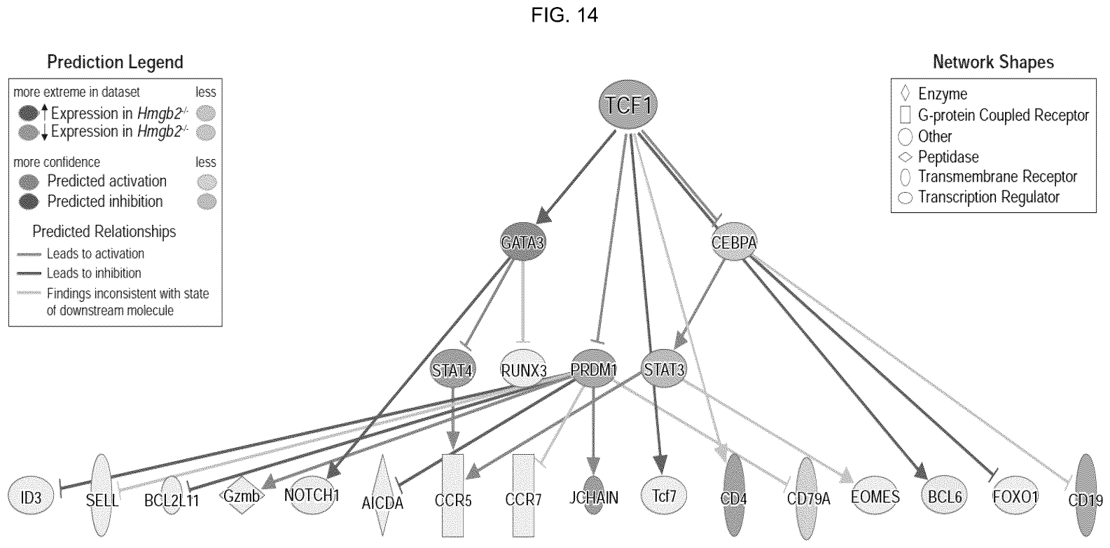

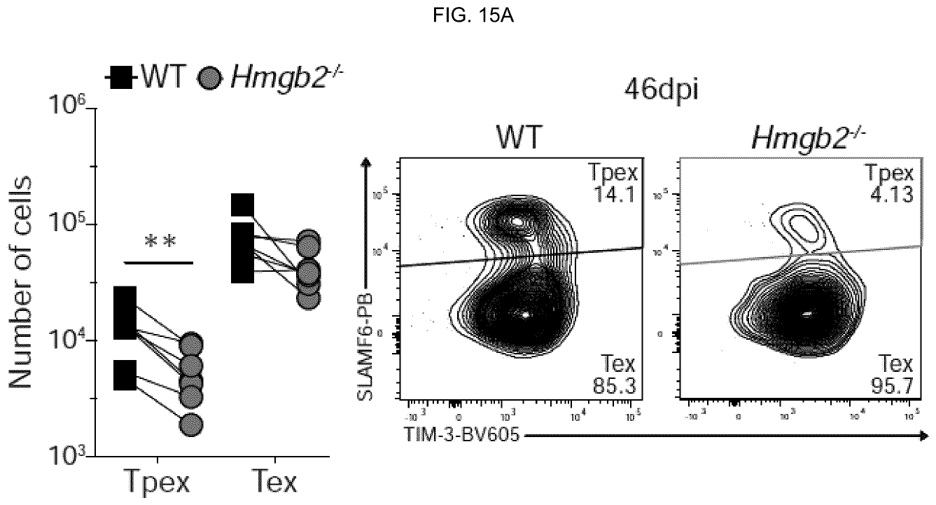

FIG. 14, or Supplementary FIG. 7: HMGB2 regulates TCF-1 transcriptional network in CD8⁺ T cells during C113 infection: (a) TCF-1 causal network identified with Ingenuity Pathway Core Analysis (IPA) (activation z-score=−3.236, network bias-corrected p-value=0.00001). Input was differentially expressed genes (DEG) (padj≤0.1, |log₂FC|≥0.5) between WT and Hmgb2^{-/-} P14 T cells at 20 dpi C113.

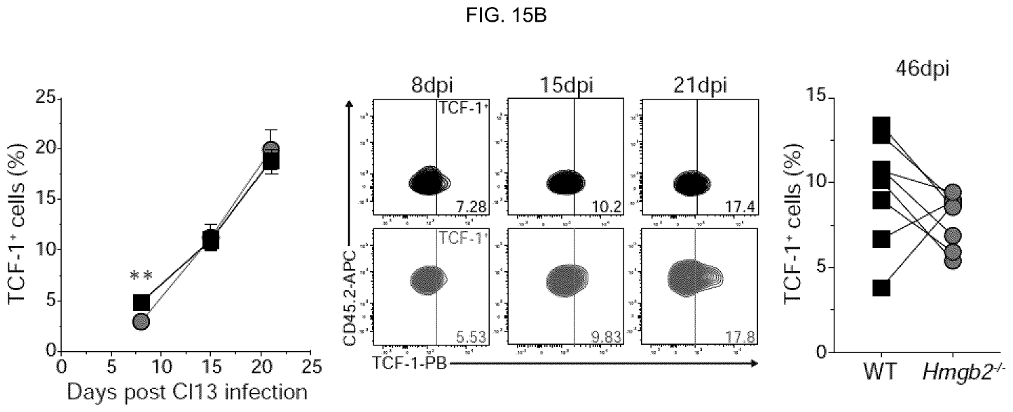

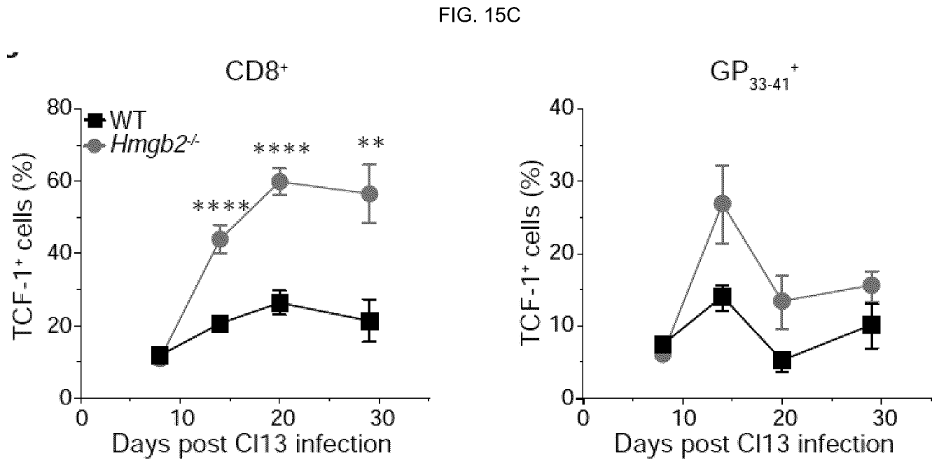

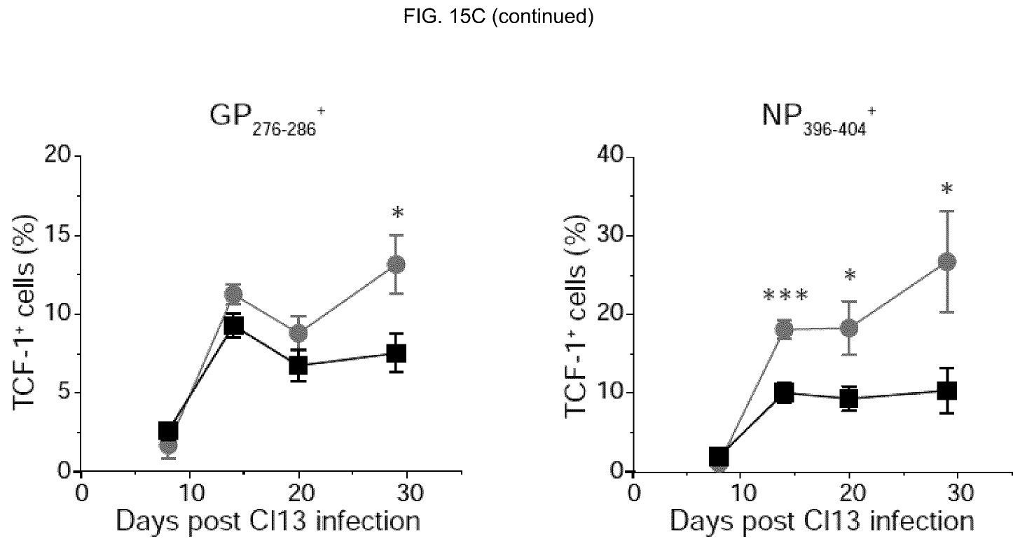

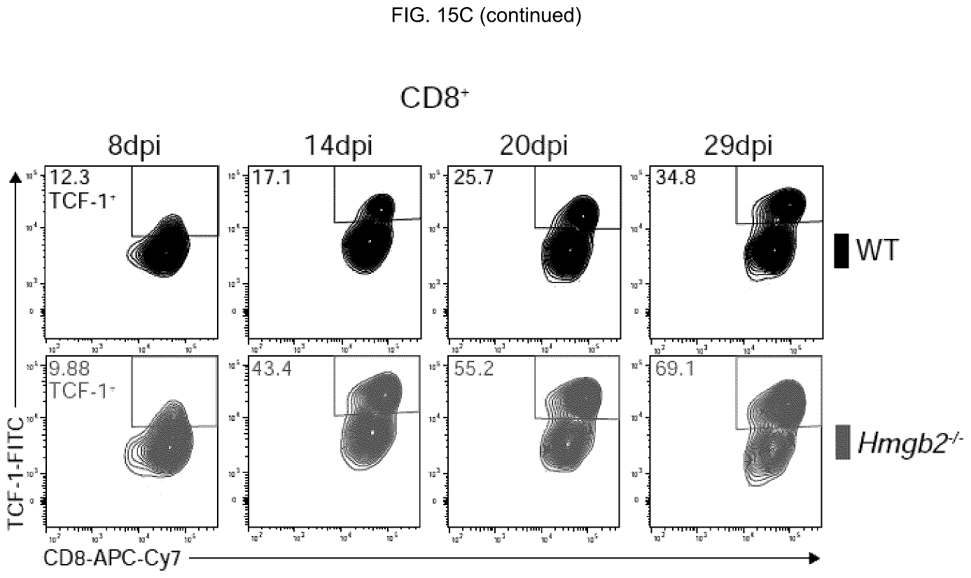

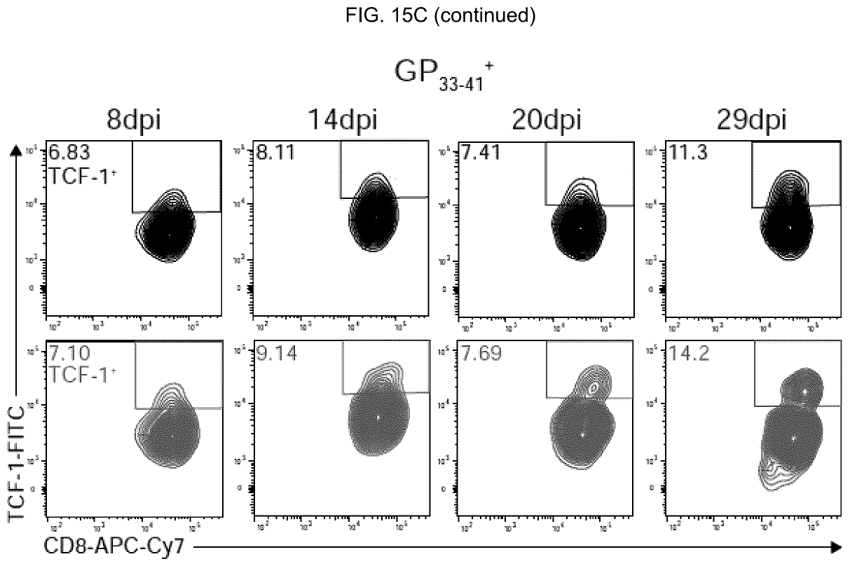

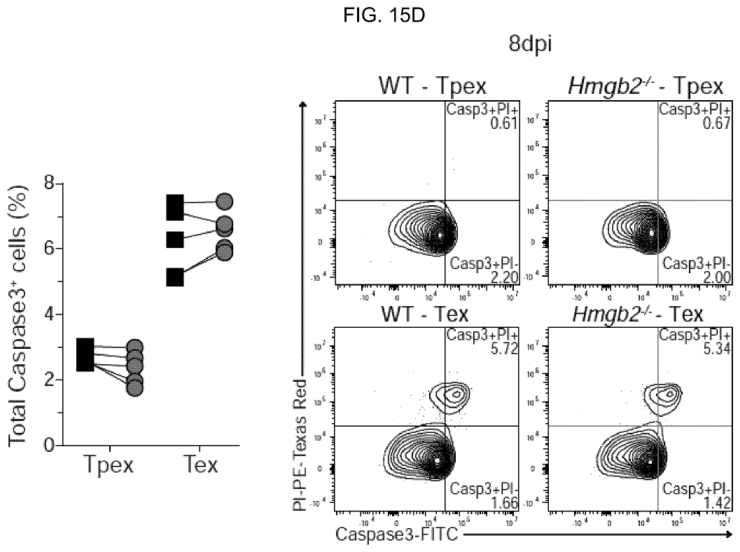

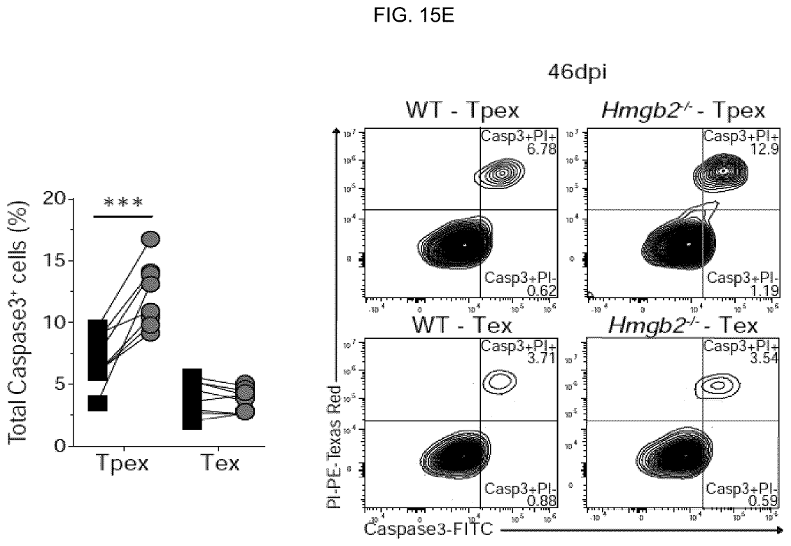

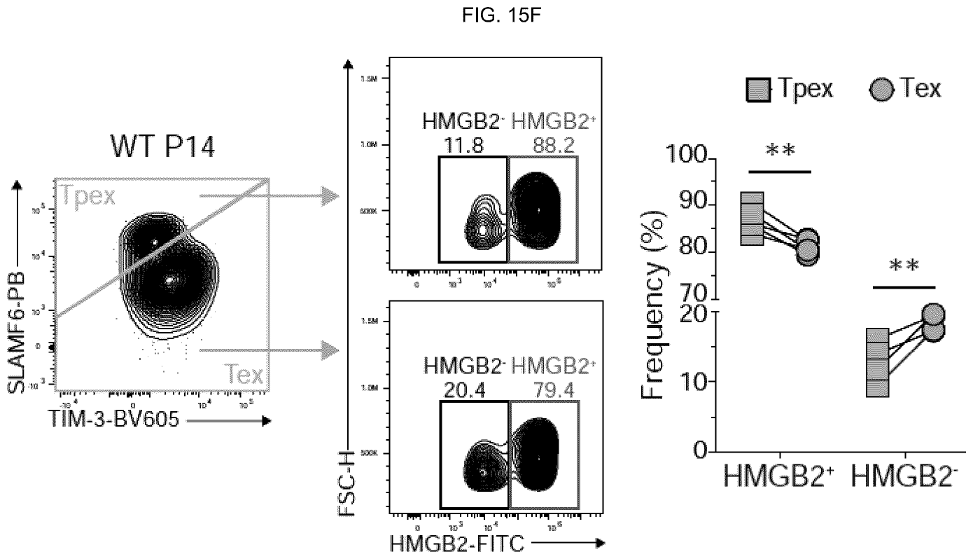

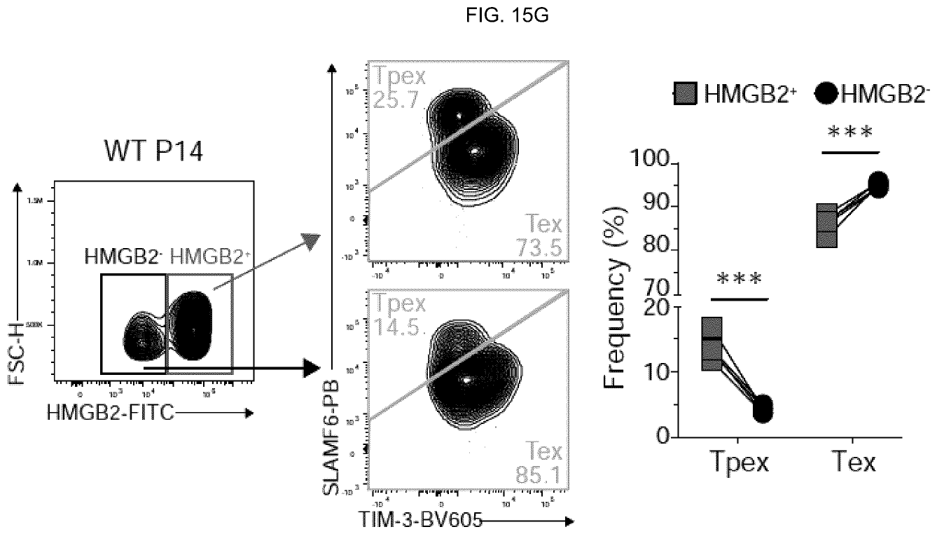

FIG. 15, or Supplementary FIG. 5. Survival and transcription factor expression in exhausted Hmgb2^{-/-} P14 T cells: P14 progenitor exhausted (Tpex) and terminal exhausted (Tex) T cell numbers at 46 dpi C113 (a). TCF-1 expression in WT and Hmgb2^{-/-} mice during C113 infection (b). TCF-1 expression in CD8⁺ and Tetramer⁺CD8⁺ T cells in WT or Hmgb2^{-/-} mice infected with C113 (c). Frequency of total Caspase3⁺ splenic WT and Hmgb2^{-/-} Tpex and Tex cells at 8 dpi (d) and 46 dpi (e) C113. Frequencies of HMGB2⁺ and HMGB2⁻ cells within progenitor exhausted (Tpex) and terminal exhausted (Tex) WT P14 T cells isolated from spleens at 8 dpi C113 (f) Frequencies of Tpex and Tex within HMGB2⁺ and HMGB2⁻ WT P14 T cells isolated from spleens at 8 dpi C113 (g). Data are representative of two independent experiments with ≥3 mice per group. Data is mean±s.e.m. *p≤0.05, **p≤0.01, ***p≤0.001, ****p≤0.0001, Paired Student's t-test, two-sided Student's t-test.

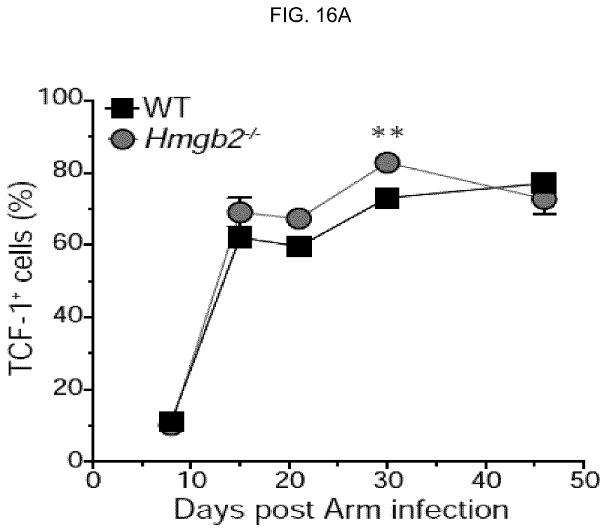

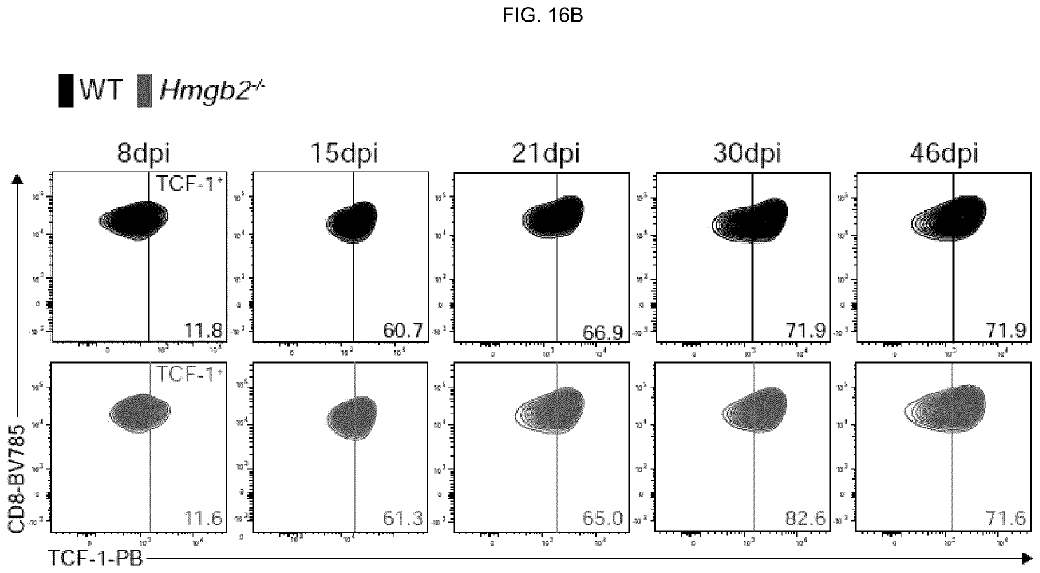

FIG. 16, or Supplementary FIG. 8. TCF-1 expression in CD8⁺ T cells during Arm infection: Frequencies of TCF-1 expression in WT or Hmgb2^{-/-} P14⁺ CD8⁺ T cells after ARM infection (a) and representative FACS plots (b). Data are representative of two independent experiments with ≥5 mice per group. Data is mean±s.e.m. **p≤0.01, ***p≤0.001, Paired Student's t-test.

### Materials and Methods

### Mice

All experimental animal procedures were approved by the Institutional Animal Care and Use Committee of University of California, Irvine (AUP-21-124) and complied with all relevant ethical regulations for animal testing and research. C57BL/6J and B6.SJL-Ptpr^{a} Pepc^{b}/BoyJ mice were purchased from the Jackson Laboratory, then bred in SPF facilities. P14 mice were obtained from The Scripps Research Institute (originally from Dr. Charles D. Surh). These mice were bred to Ly5.1 (B6.SJL-Ptprca Pepc^{b}/BoyJ) mice and to Hmgb2^{-/-} mice, which were generously provided by Dr. Marco Bianchi (San Raffaele Scientific Institute, Milan, Italy). Male and female mice≥6 weeks of age were used in experiments. Mouse selection for experiments was not formally randomized or blinded.

### Virus Infection and Titers

LCMV Armstrong (Arm) and Clone 13 (C113) strains were propagated in baby-hamster kidney cells and titrated on Vero African-green-monkey kidney cells. Frozen stocks were diluted in Vero cell media and 2×10⁵ plaque-forming units (PFUs) of LCMV Arm were injected intraperitoneally (i.p.) and 2×10⁶ PFUs of LCMV C113 were injected intravenously (i.v.). Virus titers were determined from serial dilutions of either sera or tissues taken from mice using a plaque assay.

T cell Adoptive Transfer

Bulk CD8⁺ T cells were enriched from spleens and lymph nodes (LNs) of WT (Hmgb2^{+/+}) or Hmgb2^{-/-} P14 transgenic mice by column-free magnetic negative selection. Single cell suspensions from pooled spleen and LNs were incubated with biotinylated antibodies against CD4 (GK1.5), B220 (RA3-6B2), CD19 (6D5), CD24 (M1/69), CD11b (M1/70), and CD11c (N418). Non-CD8⁺ cells were removed by mixing labeled cell suspension with Streptavidin RAPIDSPHERES™ (Stemcell technologies) at room temperature (RT) for 5 min, followed by two-5 min incubations in an EASYEIGHTS™ EASYSEP™ MAGNET™ (Stemcell technologies). The unbound CD8⁺ T cells were washed in sterile PBS (lx) with FBS (2%), and purity was determined on a flow cytometer. For single-transfer studies, WT and Hmgb2^{-/-} P14 T cells were transferred into separate new WT hosts of the opposite congenic marker (1×10³ i.v.). For co-transfer studies, WT and Hmgb2^{-/-} P14 T cells were mixed at a 1:1 ratio (1×10³ i.v. per cell-type for virus studies, 1×10⁶ i.v. per cell-type for tumor studies) and injected into new WT recipient mice i.v. Within 18-24 hr post-transfer, recipient mice were inoculated with LCMV Arm (2×10⁵, i.p.), LCMV C113 (2×10⁶ PFU, i.v.), or B16-GP₃₃₋₄₁ tumor cells (1×10⁶, s.c.). For re-challenge experiments, live (PI⁻) WT and Hmgb2^{-/-} P14 T cells were sorted at >95% purity from spleens and LNs at 30 dpi or 68 dpi using a BD FACSAria sorter. Cell numbers were normalized and transferred into new hosts (2×10³ i.v. per cell-type) that were subsequently infected with LCMV Arm (2×10⁵ PFU, i.p.).

### B16-GP₃₃₋₄₁ Tumor Model and Digestion

B16-GP₃₃₋₄₁ melanoma cells were kindly provided by Dr. Ananda Goldrath. All cell lines maintained in Iscove's Modified Dulbecco's medium supplemented with 10% fetal bovine serum (FBS) and antibiotics. All cell lines were free of mycoplasma. For tumor growth experiments, mice were injected subcutaneously (s.c.) with 1×10⁶ tumor cells. Tumor size was measured by caliper twice a week for calculation of B16-GP₃₃₋₄₁ tumor volume. Tumors were weighed at time of excision before being minced and digested in gentleMACS™ C Tubes for 40 min at 37° C. using the GENTLEMACS™ dissociator (Miltenyi Biotec). Digests were then passed through a 70-μm cell strainer to generate a single-cell suspension. The cells were then stained for flow cytometry.

### Flow Cytometry

For cell surface staining, 2×10⁶ cells were incubated with antibodies in staining buffer (PBS, 2% FBS and 0.01% NaN₃) at 4° C. For tetramer surface staining, 2×10⁶ cells were stained with conjugated H-2D^{b}-GP₃₃₋₄₁, H-2D^{b}-GP₂₇₆₋₂₈₆, or H-2D^{b}-NP₃₉₆₋₄₀₄ tetramers (NIH core facility) for 1 h and 15 min at RT in staining buffer. For intracellular cytokine staining, cells were resuspended in complete RPMI-1640 (containing 10 mM HEPES, 1% nonessential amino acids and L-glutamine, 1 mM sodium pyruvate, 10% heat inactivated FBS and antibiotics) supplemented with 50 U/mL IL-2 (NCI) and 1 mg/mL brefeldin A (BFA, Sigma), and then incubated with 2 mg/mL LCMV GP₃₃₋₄₁ peptide (AnaSpec) at 37° C. for 4 h. Cells were then fixed and permeabilized using a CYTOFIX™ CYTOPERM KIT™ (BD Biosciences) before staining. For intranuclear transcription factor staining, cells were fixed and permeabilized using a Foxp3/transcription factor fixation/permeabilization kit (Fisher). Antibodies are listed in Star Methods. Surface stains were performed at a 1:200 dilution, while intracellular and intranuclear stains performed at a 1:100 dilution. Caspase3 staining was done using CASPGLOW™ Fluorescein Active Caspase-3 staining kit (THERMOFISHER™) following manufacturer's instructions. All data were collected on a NOVOCYTE3000™ (Agilent) and analyzed using FLOWJO™ software (Tree Star).

### Imaging Flow Cytometry

For imaging flow cytometry, negative selection was performed (above) to isolate CD8⁺ T cells and cells were stained as described previously. Zombie staining was done using ZOMBIE AQUA™ FIXABLE VIABILITY KIT™ (Biolegend) as outlined by manufacturer's instructions. 7-ADD (Fisher) was used to stain nuclei per manufacturer's instructions. Cells were resuspended at 2×10⁷ cells/mL and run on an AMNIS IMAGESTREAM X MARK II™ imaging flow cytometer (EMID Millipore) and analyzed using IDEAS™ software (EMD Millipore).

### In Vivo Proliferation

Mice were injected i.p. with 2 mg BrdU (Sigma-Aldrich) 16 h before removing spleens at 8 dpi to measure proliferation. Cells were stained intracellularly using FITC BrdU Flow kit (BD Biosciences) following the manufacturer's instructions. Cells were acquired with a Novocyte3000 flow cytometer.

### Comet Assay

Co-transferred live (PI⁻) WT and Hmgb2^{-/-} P14 T cells were sorted at >95% purity from spleens and LNs of LCMV Arm or C113 infected mice at 8 dpi using a BD FACSARIA™ sorter. U2OS cells were cultured in DMEM supplemented with 10% fetal bovine serum, 1% L-Glutamine, and 1% penicillin/streptomycin. Single-cell alkaline gel electrophoresis was performed with comet assay Kit (Abcam) following manufacturer's instructions. Images were captured using a Leica DMi8 THUNDER™ microscope. Comet olive tail moments of 100 cells were analyzed using COMETSCORE™ software version 2.0.0.38™

### Western Blot

Co-transferred live (PI⁻) WT and Hmgb2^{-/-} P14 T cells were sorted at >95% purity from spleens and LNs of LCMV Arm or C113 infected mice at 8 dpi using a BD FACSAria sorter. Cells were lysed and blots were stained for Phospho-H2AX (Ser139) (1:1000) and Histone H3 (1:140000).

### Bulk RNA-Seq RNA Isolation and Library Preparation

For 8 dpi studies, WT and Hmgb2^{-/-} P14 T cells were transferred into five separate mice each before infection with either LCMV Arm or C113. For 20 dpi studies, WT and Hmgb2^{-/-} P14 T cells were co-transferred at 1:1 into 10 mice before infection with LCMV C113. On 8 dpi or 20 dpi, spleens and LNs were pooled based on infection type and P14 genotype. Live (PI⁻) WT and Hmgb2^{-/-} P14 were sorted at greater than 95% purity (8 dpi: approximately 1×10⁶ per condition; 20 dpi: approximately 300 k WT, approximately 100 k Hmgb2^{-/-}) and resuspended in RLT Buffer and BME before storage at −80° C. Each experiment was performed three times to represent three biological replicates. Total RNA was monitored for quality control using the AGILENT BIOANALYZER NANO RNA CHIP AND NANODROP™ absorbance ratios for 260/280 nm and 260/230 nm. Library construction was performed according to the ILLUMINA TRUSEQ™ mRNA stranded protocol. The input quantity for total RNA within the recommended range and mRNA was enriched using oligo dT magnetic beads. The enriched mRNA was chemically fragmented. First strand synthesis used random primers and reverse transcriptase to make cDNA. After second strand synthesis the ds cDNA was cleaned using AMPURE XP™ beads and the cDNA was end repaired and then the 3′ ends were adenylated. Illumina barcoded adapters were ligated on the ends and the adapter ligated fragments were enriched by nine cycles of PCR. The resulting libraries were validated by qPCR and sized by AGILENT BIOANALYZER™ DNA high sensitivity chip. The concentrations for the libraries were normalized and then multiplexed together. The multiplexed libraries were sequenced using paired end 100 cycles chemistry on the NOVASEQ 6000™

### Bulk RNA-Seq Data Analysis

Post-processing of the run to generate FASTQ files was performed at the Institute for Genomics and Bioinformatics (UCI IGB). PcaHubert was used to identify any outlier samples, which were removed from further analysis⁶⁷. The quality of the sequencing was first assessed using thefastQC tool (v0.11.9). Raw reads were then quality trimmed and filtered by a length of 20 bases using trimmomatic (v0.39). Trimmed reads were analyzed with the mouse Grcm38 reference genome using pseudo aligner SALMON™ (v1.2.1™) and resulting quantification files were imported using R package tximport to get TPM values for all annotated mouse genes. Differential analysis was done using R package DESeq2 (v1.22.2) with an FDR cut off of 0.05. PCA was done using R packages DESeq2 and pheatmap. For downstream analysis, genes with adjusted p-value≤0.1 and |log₂ FC|≥0.5 were included. Gene ontology functional enrichment of gene expression changes in WT and Hmgb2^{-/-} P14 T cells were performed using METASCAPE™.

### ATAC-Seq Library Preparation

WT and Hmgb2^{-/-} P14 T cells were co-transferred at 1:1 into 10 mice before infection with LCMV C113. On 8 dpi, spleens and LNs were pooled (samples are pooled from 10 mice/group). Live (PI⁻) WT and Hmgb2^{-/-} P14 were sorted at >95% purity (2×10⁵ WT, 2×10⁵ Hmgb2^{-/-}). Each experiment was performed three times to represent three biological replicates. Following the Omni-ATAC protocol, samples were lysed in lysis buffer (10 mM Tris-HCl (pH 7.4), 10 mM NaCl, 3 mM MgCl₂, 10% Np-40, 10% Tween, and 1% Digitonin) on ice for 3 minutes⁶⁸. Immediately following lysis, nuclei were spun at 500 g for 10 min at 4° C. to remove supernatant. Nuclei were then incubated with Tn5 transposase for 30 min at 37° C. Tagmented DNA was purified using AMPURE XP™ beads and PCR was performed to amplify the library under the following conditions: 72° C. for 5 min; 98° C. for 30 s; 5 cycles of 98° C. for 10 s, 63° C. for 30 s, and 72° C. for 1 min; hold at 4° C. Libraries were then purified with warm AMPURE XP™ beads and quantified on a BIOANALYZER™. Libraries were multiplexed and sequenced to a depth of 50 million 100 base pairs (bp) paired reads on a NEXTSEQ™.

### ATAC-Seq Data Analysis

Paired ended reads from sequencing were QC analyzed with FASTQQC™ (fastqQC) (v.11.9) and aligned to mouse mm10 reference genome using bowtie2 (v2.4.1). Mitochondrial reads and reads mapped to dark list (ENCODE™ Stanford version) were excluded from the downstream analysis. Duplicated reads were removed using PICARD™ tools (v2.27.1™). A union peak list was created by merging processed reads from all samples and then calling peaks using MACS2™ (v2.7.1) (-q 0.01 --keep-dup all -f BAMPE). The number of reads in each peak were then counted using FEATURECOUNTS™ (Rsubread v2.6.4) to create a counts matrix. Normalization of counts matrix was performed using DESeq2 (v1.32.0). Differentially expressed peaks were determined using EDGER™ (v3.34.1) with an FDR cut-off of 0.05 and a |log₁₀FC| cut-off of ≥0.3. Peaks were annotated using CHIPSEEKER™ (v1.34.0). Functional enrichment of promoter regions was performed using METASCAPE™.

### Human HCV Data Analysis

Low-input RNA sequencing of HCV-specific CD8⁺ T cells was performed by Hensel et al., 2021 (GEO #GSE150345)²². In short, transcriptome analyses of sorted HCV-specific CD8⁺ T cells isolated from patients was performed using an Illumina NextSeq 500 platform and RNA reads were aligned to the human reference genome. For our studies, post-processing of the low-input sequencing data was performed at the UCI IGB. Gene expression levels were compared in chronically infected HCV patients pre- and post-DAA cure using differential analysis with multi factor design of paired samples in DESeq2⁶⁹.

### Quantification and Statistical Analysis

Flow cytometry data were analyzed with FLOWJO™ software (TreeStar). Bulk RNA-seq and ATAC-seq figures were prepared using RSTUDIO™ software. Graphs were prepared with GRAPHPAD PRISM™ software. GRAPHPAD PRISM™ was used for statistical analysis to compare outcomes using a two-tailed unpaired Student's t-test, Mann Whitney or paired Student's t-test where indicated; significance was set to p≤0.05 and represented as *p≤0.05, **p≤0.01, ***p≤0.001, and ****p≤0.0001. Error bars show SEM.

### Data and Code Availability

The authors declare that all supporting data are available within the Article and its Supplementary Information files. 3′-scRNA-seq and ATAC-seq data sets will be deposited in the GENE EXPRESSION OMNIBUS™ (GEO) database (NCBI, NIH) under the accession code GSE237813.

### REFERENCES

1. Barber, D. L. et al. Restoring function in exhausted CD8 T cells during chronic viral infection. Nature 439, 682-687 (2006).
2. Utzschneider, D. T. et al. T Cell Factor 1-Expressing Memory-like CD8(⁺) T Cells Sustain the Immune Response to Chronic Viral Infections. Immunity 45, 415-427 (2016).
3. Schmitz, J. E. et al. Control of viremia in simian immunodeficiency virus infection by CD8⁺ lymphocytes. Science 283, 857-860 (1999).
4. Franco, F., et al. Metabolic and epigenetic regulation of T-cell exhaustion. Nat Metab 2, 1001-1012 (2020).
5. Zeng, Z., et al. Exhausted T cells and epigenetic status. Cancer Biol Med 17, 923-936 (2020).
6. Speiser, D. E. et al. T cell differentiation in chronic infection and cancer: functional adaptation or exhaustion? Nat Rev Immunol 14, 768-774 (2014).
7. Scott-Browne, J. P. et al. Dynamic Changes in Chromatin Accessibility Occur in CD8⁺ T Cells Responding to Viral Infection. Immunity 45, 1327-1340 (2016).
8. Sen, D. R. et al. The epigenetic landscape of T cell exhaustion. Science 354, 1165-1169 (2016).
9. Im, S. J. et al. Defining CD8⁺ T cells that provide the proliferative burst after PD-1 therapy. Nature 537, 417-421 (2016).
10. Miller, B. C. et al. Subsets of exhausted CD8⁺ T cells differentially mediate tumor control and respond to checkpoint blockade. Nature Immunology 20, 326-336 (2019).
11. Utzschneider, D. T. et al. Early precursor T cells establish and propagate T cell exhaustion in chronic infection. Nature Immunology 21, 1256-1266 (2020).
12. Kallies, A., Zehn, D. & Utzschneider, D. T. Precursor exhausted T cells: key to successful immunotherapy? Nat Rev Immunol 20, 128-136 (2020).
13. McLane, L. M., et al., CD8 T Cell Exhaustion During Chronic Viral Infection and Cancer. Annual Review of Immunology 37, 457-495 (2019).
14. Sade-Feldman, M. et al. Defining T Cell States Associated with Response to Checkpoint Immunotherapy in Melanoma. Cell 175, 998-1013.e1020 (2018).
15. Lau, J. S. Y. et al. The impact of immune checkpoint therapy on the latent reservoir in HIV-infected individuals with cancer on antiretroviral therapy. AIDS 35, 1631-1636 (2021).
16. Rutishauser, R. L. et al. TCF-1 regulates HIV-specific CD8⁺ T cell expansion capacity. JCI Insight 6 (2021).
17. Wong, W. K. et al. The Interplay Between Epigenetic Regulation and CD8(⁺) T Cell Differentiation/Exhaustion for T Cell Immunotherapy. Front Cell Dev Biol 9, 783227 (2021).
18. Pauken, K. E. et al. Epigenetic stability of exhausted T cells limits durability of reinvigoration by PD-1 blockade. Science 354, 1160-1165 (2016).
19. Utzschneider, D. T. et al. T cells maintain an exhausted phenotype after antigen withdrawal and population reexpansion. Nature Immunology 14, 603-610 (2013).
20. Das, S. & Johnson, D. B. Immune-related adverse events and anti-tumor efficacy of immune checkpoint inhibitors. J Immunother Cancer 7, 306 (2019).
21. Abdel-Hakeem, M. S. et al. Epigenetic scarring of exhausted T cells hinders memory differentiation upon eliminating chronic antigenic stimulation. Nat Immunol 22, 1008-1019 (2021).
22. Hensel, N. et al. Memory-like HCV-specific CD8(⁺) T cells retain a molecular scar after cure of chronic HCV infection. Nat Immunol 22, 229-239 (2021).
23. Yates, K. B. et al. Epigenetic scars of CD8(⁺) T cell exhaustion persist after cure of chronic infection in humans. Nat Immunol 22, 1020-1029 (2021).
24. Ueda, T. & Yoshida, M. HMGB proteins and transcriptional regulation. Biochimica et Biophysica Acta (BBA)—Gene Regulatory Mechanisms 1799, 114-118 (2010).
25. Mallik, R., Kundu, A. & Chaudhuri, S. High mobility group proteins: the multifaceted regulators of chromatin dynamics. Nucleus 61, 213-226 (2018).
26. Štros, M., et al., The HMG-box: a versatile protein domain occurring in a wide variety of DNA-binding proteins. Cell. Mol. Life Sci. 64, 2590 (2007).
27. Benavides Damm, T. & Egli, M. Calcium's role in mechanotransduction during muscle development. Cell. Physiol. Biochem. 33, 249-272 (2014).
28. Ronfani, L. et al. Reduced fertility and spermatogenesis defects in mice lacking chromosomal protein Hmgb2. Development 128, 1265-1273 (2001).
29. Abraham, A. B. et al. Members of the high mobility group B protein family are dynamically expressed in embryonic neural stem cells. Proteome Sci 11, 18 (2013).
30. Wherry, E. J. et al. Molecular signature of CD8⁺ T cell exhaustion during chronic viral infection. Immunity 27, 670-684 (2007).
31. Mo, Y. et al. MicroRNA-329 upregulation impairs the HMGB2/beta-catenin pathway and regulates cell biological behaviors in melanoma. J Cell Physiol 234, 23518-23527 (2019).
32. Lou, N., et al., High-mobility group box 2 reflects exacerbated disease characteristics and poor prognosis in non-small cell lung cancer patients. Ir J Med Sci (2021).
33. Li, H. et al. Dysfunctional CD8 T Cells Form a Proliferative, Dynamically Regulated Compartment within Human Melanoma. Cell 181, 747 (2020).
34. Kaczanowska, S. et al. Genetically engineered myeloid cells rebalance the core immune suppression program in metastasis. Cell 184, 2033-2052 e2021 (2021).
35. Milner, J. J. et al. Delineation of a molecularly distinct terminally differentiated memory CD8 T cell population. Proc Natl Acad Sci USA 117, 25667-25678 (2020).
36. Alfei, F. et al. TOX reinforces the phenotype and longevity of exhausted T cells in chronic viral infection. Nature 571, 265-269 (2019).
37. Khan, O. et al. TOX transcriptionally and epigenetically programs CD8⁺ T cell exhaustion. Nature 571, 211-218 (2019).
38. Seo, H. et al. TOX and TOX2 transcription factors cooperate with NR4A transcription factors to impose CD8⁺ T cell exhaustion. PNAS 116, 12410-12415 (2019).
39. Dolina, J. S., Van Braeckel-Budimir, N., Thomas, G. D. & Salek-Ardakani, S. CD8(⁺) T Cell Exhaustion in Cancer. Front Immunol 12, 715234 (2021).
40. Kramer, A., Green, J., Pollard, J., Jr. & Tugendreich, S. Causal analysis approaches in Ingenuity Pathway Analysis. Bioinformatics 30, 523-530 (2014).
41. Olive, P. L. & Banath, J. P. The comet assay: a method to measure DNA damage in individual cells. Nat Protoc 1, 23-29 (2006).
42. Mah, L. J., El-Osta, A. & Karagiannis, T. C. gammaH2AX: a sensitive molecular marker of DNA damage and repair. Leukemia 24, 679-686 (2010).
43. Shan, Q. et al. Tcf1 preprograms the mobilization of glycolysis in central memory CD8(⁺) T cells during recall responses. Nat Immunol 23, 386-398 (2022).
44. Jadhav, R. R. et al. Epigenetic signature of PD-1⁺ TCF 1⁺ CD8 T cells that act as resource cells during chronic viral infection and respond to PD-1 blockade. Proc Natl Acad Sci USA 116, 14113-14118 (2019).
45. Hudson, W. H. et al. Proliferating Transitory T Cells with an Effector-like Transcriptional Signature Emerge from PD-1(⁺) Stem-like CD8(⁺) T Cells during Chronic Infection. Immunity 51, 1043-1058 e1044 (2019).
46. Beltra, J. C. et al. Developmental Relationships of Four Exhausted CD8(⁺) T Cell Subsets Reveals Underlying Transcriptional and Epigenetic Landscape Control Mechanisms. Immunity 52, 825-841 e828 (2020).
47. Kaech, S. M. & Ahmed, R. Memory CD8⁺ T cell differentiation: initial antigen encounter triggers a developmental program in naïve cells. Nat Immunol 2, 415-422 (2001).
48. Kaech, S. M. & Cui, W. Transcriptional control of effector and memory CD8⁺ T cell differentiation. Nat Rev Immunol 12, 749-761 (2012).
49. Kaech, S. M. et al. Selective expression of the interleukin 7 receptor identifies effector CD8 T cells that give rise to long-lived memory cells. Nat Immunol 4, 1191-1198 (2003).
50. Yuzefpolskiy, Y., Baumann, F. M., Kalia, V. & Sarkar, S. Early CD8 T-cell memory precursors and terminal effectors exhibit equipotent in vivo degranulation. Cell Mol Immunol 12, 400-408 (2015).
51. Wherry, E. J. et al. Lineage relationship and protective immunity of memory CD8 T cell subsets. Nat Immunol 4, 225-234 (2003).
52. Jameson, S. C. & Masopust, D. Understanding Subset Diversity in T Cell Memory. Immunity 48, 214-226 (2018).
53. Jeannet, G. et al. Essential role of the Wnt pathway effector Tcf-1 for the establishment of functional CD8 T cell memory. Proc Natl Acad Sci USA 107, 9777-9782 (2010).
54. Zhou, X. et al. Differentiation and persistence of memory CD8(⁺) T cells depend on T cell factor 1. Immunity 33, 229-240 (2010).
55. Aliahmad, P. et al. TOX provides a link between calcineurin activation and CD8 lineage commitment. J Exp Med 199, 1089-1099 (2004).
56. Laurent, B. et al. High-mobility group protein HMGB2 regulates human erythroid differentiation through trans-activation of GFI1B transcription. Blood 115, 687-695 (2010).
57. Abraham, A. B. et al. Aberrant neural stem cell proliferation and increased adult neurogenesis in mice lacking chromatin protein HMGB2. PLoS One 8, e84838 (2013).
58. Fang, Y. et al. High mobility group box 2 regulates skeletal muscle development through ribosomal protein S6 kinase 1. FASEB J 34, 12367-12378 (2020).
59. Taniguchi, N. et al. Chromatin protein HMGB2 regulates articular cartilage surface maintenance via beta-catenin pathway. Proc Natl Acad Sci USA 106, 16817-16822 (2009).
60. Zhang, C. et al. Latexin regulation by HMGB2 is required for hematopoietic stem cell maintenance. 1 105, 573-584 (2020).
61. Wu, T. et al. The TCF1-Bcl6 axis counteracts type I interferon to repress exhaustion and maintain T cell stemness. Sci Immunol 1 (2016).
62. Tabana, Y., et al., Reversing T-cell exhaustion in immunotherapy: a review on current approaches and limitations. Expert Opin Ther Targets 25, 347-363 (2021).
63. Zhang, Z. et al. T Cell Dysfunction and Exhaustion in Cancer. Front Cell Dev Biol 8, 17 (2020).
64. Gearty, S. V. et al. An autoimmune stem-like CD8 T cell population drives type 1 diabetes. Nature (2021).
65. Xing, S. et al. Tcf1 and Lef1 are required for the immunosuppressive function of regulatory T cells. J Exp Med 216, 847-866 (2019).
66. Shan, Q. et al. Tcf1 and Lef1 provide constant supervision to mature CD8(⁺) T cell identity and function by organizing genomic architecture. Nat Commun 12, 5863 (2021).
67. Chen, X., et al., Robust principal component analysis for accurate outlier sample detection in RNA-Seq data. BMC Bioinformatics 21, 269 (2020).
68. Corces, M. R. et al. An improved ATAC-seq protocol reduces background and enables interrogation of frozen tissues. Nat Methods 14, 959-962 (2017).
69. Love, M. I., et al., Moderated estimation of fold change and dispersion for RNA-seq data with DESeq2. Genome Biol 15, 550 (2014).

A number of embodiments of the invention have been described. Nevertheless, it can be understood that various modifications may be made without departing from the spirit and scope of the invention. Accordingly, other embodiments are within the scope of the following claims.

What is claimed is:

1. A method for:
increasing the rate or amount of differentiation and stemness of memory and exhausted CD8⁺ T cells in vivo,
increasing the rate or amounts of effector and memory CD8⁺ T cells in vivo;
expanding a subset of progenitor stem cells within an exhausted T cell population in vivo or ex vivo;
boosting or increasing CD8⁺ T cells during acute viral infection in an individual in need thereof,
increasing memory T cells and memory T cells differentiation, memory precursor effector (MPEC) and central memory (Tcm) T cell phenotypes, and memory recall responses in vivo,
increasing the ability of an individual in need thereof to recover from a viral infection, and/or
treating, ameliorating, slowing the progression of, decreasing the severity of symptoms of, an infection, optionally, a viral infection, in an individual in need thereof,
comprising:
comprising administering to an individual in need thereof:
a nucleic acid encoding a HMGB2 (high-mobility group box2) protein, or
a T cell, optionally a CD8⁺ T cell, genetically manipulated or engineered to have increased HMGB2 activity,
wherein optionally the HMGB2-expressing nucleic acid comprises the nucleic acid sequence (SEQ ID NO:1):
or a nucleic acid having between about 90% to 99% sequence identity to SEQ ID NO:1,
and optionally the HMGB2-expressing nucleic acid comprises a nucleic acid sequence that encodes the polypeptide (SEQ ID NO:2):
or a polypeptide having between about 90% to 99% sequence identity to SEQ ID NO:2, or a polypeptide having about 92%, 95% or 97% sequence identity to SEQ ID NO: 2.

2. A method for:
decreasing the rate or amount of differentiation and sternness of memory and exhausted CD8⁺ T cells in vivo,
decreasing the rate or amounts of effector and memory CD8⁺ T cells in vivo;
expanding a subset of progenitor stem cells within an exhausted T cell population in vivo or ex vivo; and/or
treating, ameliorating, slowing the progression of, decreasing the severity of symptoms of, an autoimmune disease, in an individual in need thereof,
comprising:
comprising administering to an individual in need thereof:
a nucleic acid or HMGB2-inhibiting molecule capable of decreasing the activity or expression of a HMGB2 (high-mobility group box2) protein, or
wherein optionally the HMGB2-expressing nucleic acid comprises the nucleic acid sequence (SEQ ID NO:1):
or a nucleic acid having between about 90% to 99% sequence identity to SEQ ID NO:1,
and optionally the HMGB2-expressing nucleic acid comprises a nucleic acid sequence that encodes the polypeptide (SEQ ID NO:2):
or a polypeptide having between about 90% to 99% sequence identity to SEQ ID NO:2, or a polypeptide having about 92%, 95% or 97% sequence identity to SEQ ID NO:2.

3. The method of claim 2, wherein the HMGB2-encoding nucleic acid, or the HMGB2-inhibiting nucleic acid, is contained in an expression construct, a plasmid, an expression vehicle, a virus or a vector, and the expression construct, plasmid, expression vehicle, virus or vector is delivered or administered to the individual in need thereof,
wherein optionally the expression vehicle or vector is selected from the group consisting of a herpes simplex virus, a human immunodeficiency virus (HIV), a synthetic vector, an adeno-associated virus (AAV), a lentivirus, an adenovirus and a plasmid,
and optionally the AAV is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.

4. The method of any of claim 1, wherein the HMGB2-expressing nucleic acid, or the expression construct, plasmid, expression vehicle, virus or vector, or the genetically manipulated or engineered T cell, is formulated:
(a) in a liquid, a gel, a hydrogel, a vesicle, a liposome, a nanoparticle, a nanolipid particle, a powder or an aqueous or a saline formulation, or for administration in vitro or in vivo;
(b) for enteral or parenteral administration;
(c) in or as a liposome, a nanoparticle, or a nanoliposome;
(d) in or as a tablet, a pill, a capsule, a gel, a hydrogel, a geltab, a liquid, a powder, an emulsion, a lotion, an aerosol, a spray, a lozenge, an aqueous or a sterile or an injectable solution, an eye drop, or an implant; or
(e) for intravenous injection, subcutaneous injection, intramuscular injection, inhalation, or intravitreal injection.

5. The method of any of claim 2, wherein the HMGB2-inhibiting nucleic acid, or the expression construct, plasmid, expression vehicle, virus or vector, or the genetically manipulated or engineered T cell, is formulated:
(a) in a liquid, a gel, a hydrogel, a vesicle, a liposome, a nanoparticle, a nanolipid particle, a powder or an aqueous or a saline formulation, or for administration in vitro or in vivo;
(b) for enteral or parenteral administration;
(c) in or as a liposome, a nanoparticle, or a nanoliposome;
(d) in or as a tablet, a pill, a capsule, a gel, a hydrogel, a geltab, a liquid, a powder, an emulsion, a lotion, an aerosol, a spray, a lozenge, an aqueous or a sterile or an injectable solution, an eye drop, or an implant; or
(e) for intravenous injection, subcutaneous injection, intramuscular injection, inhalation, or intravitreal injection.

6. The method of any of claim 2, wherein the HMGB2-inhibiting molecule is or comprises a small molecule, an inhibitory nucleic acid (optionally and miRNA or antisense molecule), polypeptide or peptide (optionally an antibody capable of specifically binding to HMGB2 protein and inhibiting its expression or activity), a lipid or a polysaccharide.

7. The method of any of claim 2, wherein the HMGB2-inhibiting nucleic acid is or comprises: an RNAi inhibitory nucleic acid molecule, a double-stranded RNA (dsRNA) molecule, a microRNA (mRNA), a small interfering RNA (siRNA), an antisense RNA, a short hairpin RNA (shRNA), an inhibitory ribozyme, or a CRISPR system such as CRISPR-Cas9.
